(19) United States
(12) Patent Application Publication
(10) Pub. No.: US 2005/0207974 A1
(43) Pub. Date: Sep. 22, 2005

(54) Endothelial cell markers and related reagents and methods of use thereof

(76) Inventors: David Xing-Fei Deng, Mountain View, CA (US); Anya Tsalenko, Chicago, IL (US); Laurakay Bruhn, Mountain View, CA (US); Zohar Yakhini, Ramat Hasharon (IL); Michael Ho, Freemont, CA (US); Thomas Quertermous, Stanford, CA (US); Eugene Yang, Mountain View, CA (US)

Correspondence Address: AGILENT TECHNOLOGIES, INC.;Legal Department, DL429, Intellectual Property Administration, P.O. Box 7599, Loveland, CO, 80537-0599, US

(21) Appl. No.: 10/802,246

(22) Filed: Mar. 17, 2004

(51) Int. Cl. A61K 51/00; C07K 16/46

(52) U.S. Cl. 424/1.49; 530/391.1; 530/400

(57) ABSTRACT

The present invention provides polynucleotides and polypeptides (VECSM polynucleotides and polypeptides) that are differentially expressed in vascular endothelial cells. The invention further provides a variety of compositions, diagnostic, and therapeutic methods based on identification of these markers. In particular, the invention provides a targeting agent linked to a functional moiety, wherein the functional moiety can comprise any of a number of different agents, including imaging agents, cytotoxic agents, and stimulators or inhibitors of angiogenesis.

### BACKGROUND OF THE INVENTION

Vascular endothelial cells maintain the interface between the systemic circulation and soft tissues and mediate critical processes such as inflammation, coagulation and hemostasis. Vascular endothelium is also involved in a diverse array of pathological conditions ranging from atherosclerosis and restenosis to diabetic nephropathy.

Tissue cells typically cannot survive beyond the area within the oxygen diffusion limit of approximately 100-200 μm around blood vessels. Angiogenesis, the formation of new blood vessels from existing vasculature, is the major mechanism that rapidly growing tissues use to meet their increased requirement for nutrients and oxygen. Angiogenesis occurs primarily during embryonic development, in which new blood vessels are first generated from endothelial precursors (vasculogenesis) and then expand the primitive tubular network by sprouting (angiogenesis). Angiogenesis also takes place in adult tissues in a tightly regulated manner under physiological conditions such as wound healing, ovulation and menstruation. In addition, angiogenesis is a hallmark of various pathological conditions such as cardiovascular ischemia, inflammatory diseases (e.g. rheumatoid arthritis, etc), and cancers.

Angiogenesis is essential for tumor progression and metastasis in many tumor types, as well as for other processes such as tissue repair in cardiovascular ischemic diseases. The close relationship between angiogenesis and cancer progression and metastasis has been documented in many clinical studies. For example, the change from breast ductal carcinoma in situ to invasive cancer is strongly associated with angiogenesis. Patient prognosis is also related to angiogenesis. Increased levels of vascular endothelial growth factor (VEGF), which is known to induce angiogenesis, are associated with poorer overall prognosis in breast cancer. Furthermore, micro-vessel density (MVD), an indicator of tumor angiogenic activity as measured by immunohistochemical staining, is an important prognostic factor independent of other known prognostic parameters, including stage, grade and lymph node status in a number of cancer types.

In recent years, angiogenesis has become a key research area for treatment of cardiovascular diseases and cancer. Promoting new blood vessels could be beneficial in patients suffering from ischemic vascular diseases, in which blood supply to certain tissues is insufficient. Inhibiting new vessel formation has applications in cancer, in which tumor growth and metastasis require new blood vessels. The effective development of therapies directed at vascular endothelium, and at angiogenic pathways, requires both the discovery of appropriate endothelial cell targets and also the development of effective methods of assessing the effects of pro- and anti-angiogenic agents. Accordingly, there is a need for the identification of additional endothelial cell specific markers and for the identification of markers indicative of angiogenesis. In addition, there is a need for the identification of marker genes that are differentially expressed in endothelial cells of various types.

### SUMMARY OF THE INVENTION

The present invention identifies endothelial marker genes whose expression occurs selectively or specifically in vascular endothelial cells, i.e., genes that are differentially expressed in endothelial cells relative to other cell types. These genes, and their associated polypeptides and polynucleotides, have been named VECSM genes, VECSM polynucleotides, and VECSM polypeptides, where VECSM stands for “vascular endothelial cell specific marker”. The VECSM genes have been classified into 3 groups. Group I includes genes previously identified as differentially expressed in endothelial cells. Group II includes genes previously characterized but not previously identified as differentially expressed in endothelial cells. Group III includes genes for which previous characterization was minimal or nonexistent. The invention provides VECSM polynucleotides, polypeptides, and a variety of related reagents and methods of use thereof.

In one aspect, the invention provides a composition comprising: (i) a targeting agent conjugated to a functional moiety, wherein the targeting agent selectively binds to a VECSM Group II or Group III polypeptide. In various embodiments of the invention the functional moiety comprises an angiogenesis inhibitor or a stimulator of angiogenesis, a radiosensitizing agent, or an imaging agent.

In additional aspects, the invention provides an siRNA or shRNA targeted to a VECSM Group II or Group III polynucleotide, an antisense molecule that specifically binds to and inhibits a VECSM Group II or Group III polynucleotide, a ribozyme that cleaves a VECSM Group II or Group III polynucleotide, and an antibody or antibody fragment that specifically binds to a VECSM Group II or Group III polypeptide, wherein the antibody detects expression of the polypeptide in endothelial cells. The invention also provides method of inhibiting expression of a VECSM Group II or Group III polypeptide in a cell or a subject comprising delivering a VECSM Group II or Group III antisense oligonucleotide, ribozyme, siRNA, or shRNA to the cell or subject. The invention further provides a method of treating a condition associated with inappropriate or excessive vascular endothelial growth comprising steps of: (i) providing a subject in need of treatment for a condition associated with inappropriate or excessive vascular endothelial growth; and (ii) administering a pharmaceutical composition comprising a VECSM Group II or Group III antisense oligonucleotide, ribozyme, siRNA, or shRNA to the subject, thereby alleviating the condition. The invention also provides a method of treating a condition associated with a reduced or inadequate blood supply comprising steps of: (i) providing a subject in need of treatment for a condition associated with a reduced or inadequate blood supply; and (ii) administering a pharmaceutical composition comprising a VECSM Group II or Group III antisense oligonucleotide, ribozyme, siRNA, or shRNA to the subject, thereby alleviating the condition.

In another aspect, the invention provides a method for detecting or quantifying vascularization or angiogenic activity in a biological sample or subject comprising: determining the level of expression of a naturally occurring VECSM Group II or Group III polynucleotide in the biological sample or subject, or determining the level of expression of a naturally occurring polypeptide encoded by such a polynucleotide in the biological sample or subject.

The invention further provides a method of imaging angiogenesis, vasculature, or body tissue comprising endothelial cells in a biological sample or subject, comprising steps of: (i) administering to the biological sample or subject an effective amount of a targeting agent that specifically binds to a VECSM Group II or Group III polypeptide, wherein the targeting agent is linked to a functional moiety that enhances detectability of endothelial cells by an imaging procedure; and (ii) subjecting the biological sample or subject to the imaging procedure.

The invention provides a method of targeting a molecule selectively to an endothelial cell or to vascular endothelium in a subject comprising steps of: (i) conjugating the molecule to an antibody or ligand that specifically binds to a VECSM Group II or Group III polypeptide to form a conjugate; and (ii) administering the conjugate to the cell or subject. In addition, the invention provides a method of targeting a molecule selectively to an endothelial cell or to vascular endothelium in a subject comprising steps of: (i) associating the molecule with a delivery vehicle, wherein the delivery vehicle comprises a targeting agent that specifically binds to a VECSM Group II or III polypeptide; and (ii) administering the delivery vehicle with the associated molecule to the cell or subject.

In another aspect, the invention provides a method of providing diagnostic or prognostic information related to a disease or condition associated with inappropriate or excessive angiogenesis or vascular endothelial growth comprising steps of: (i) providing a subject in need of diagnostic or prognostic information related to a disease or condition associated with inappropriate or excessive angiogenesis or vascular endothelial growth; (ii) determining the level of expression or activity of a VECSM Group II or Group III polynucleotide or polypeptide in the subject or in a biological sample obtained from the subject; and (iii) concluding that there is an increased likelihood that the subject is at risk of or suffering from a disease or condition associated with inappropriate or excessive angiogenesis or vascular endothelial growth if the level of expression or activity of the VECSM polynucleotide or polypeptide is higher than would be expected in a normal subject or in a biological sample obtained from a normal subject.

The invention further provides a method of providing diagnostic or prognostic information related to a disease or condition associated with an inadequate or reduced blood supply or inadequate or reduced angiogenesis or vascular endothelial growth comprising steps of: (i) providing a subject in need of diagnostic or prognostic information related to a disease or condition associated with inadequate or reduced blood supply or inadequate or reduced angiogenesis or vascular endothelial growth; (ii) determining the level of expression or activity of a VECSM Group II or Group III polynucleotide or polypeptide in the subject or in a biological sample obtained from the subject; and (iii) concluding that there is an increased likelihood that the subject is at risk of or suffering from a disease or condition associated with inadequate or reduced blood supply or inadequate or reduced angiogenesis or vascular endothelial growth if the level of expression or activity of the VECSM polynucleotide or polypeptide is lower than would be expected in a normal subject or in a biological sample obtained from a normal subject.

The invention further provides a method of inhibiting angiogenesis in a subject comprising steps of: (a) providing a subject having a condition characterized by excessive or inappropriate angiogenesis; and (b) administering a composition comprising (i) a targeting agent that specifically binds to a VECSM Group II or Group III polypeptide; and (ii) a functional moiety, wherein the functional moiety comprises an angiogenesis inhibitor, to the subject, thereby inhibiting angiogenesis in the subject. The invention also provides a method of inhibiting tumor growth or survival in a subject comprising steps of: (a) providing a subject having a tumor; and (b) administering to the subject a composition comprising (i) a targeting agent that specifically binds to a VECSM Group II or Group III polypeptide; and (ii) a functional moiety, wherein the functional moiety comprises an angiogenesis inhibitor, to the subject, thereby inhibiting growth, survival, or metastasis of the tumor. The invention also provides a method of inhibiting tumor growth or survival in a subject comprising steps of: (a) providing a subject having a tumor; and (b) administering to the subject a composition comprising (i) a targeting agent that specifically binds to a VECSM Group II or Group III polypeptide; and (ii) a functional moiety, wherein the functional moiety comprises a cytotoxic moiety, to the subject, thereby inhibiting growth, survival, or metastasis of the tumor.

In another aspect, the invention provides a method of stimulating angiogenesis in a subject comprising steps of: (a) providing a subject having a condition characterized by an inadequate blood supply to one or more organs or tissues; and (b) administering a composition comprising (i) a targeting agent that specifically binds to a VECSM Group II or Group III polypeptide; and (ii) a functional moiety, wherein the functional moiety comprises a stimulator of angiogenesis, to the subject, thereby stimulating angiogenesis in the subject.

In another aspect, the invention provides a method for identifying a compound comprising steps of: (i) providing a biological sample comprising cells that express a VECSM Group II or Group III polypeptide; (ii) contacting the cells with the compound; (iii) determining whether the level of expression or activity of the VECSM polynucleotide or polypeptide in the presence of the compound is increased or decreased relative to the level of expression or activity of the VECSM polynucleotide or polypeptide in the absence of the compound; and (iv) identifying the compound as a modulator of the VECSM polynucleotide or polypeptide if the level of expression or activity of the VECSM polynucleotide or polypeptide is higher or lower in the presence of the compound relative to its level of expression or activity in the absence of the compound.

The invention further provides a method of identifying an endothelial cell as a microvascular endothelial cell or of identifying a population of endothelial cells as a microvascular endothelial cell population comprising steps of: (i) measuring, in an endothelial cell or in a population of endothelial cells, the level or activity of an expression product of a gene that is overexpressed in microvascular endothelial cells relative to its expression in a different endothelial cell type; (ii) comparing the level of expression or activity of the expression product to the level of expression or activity expected in a cell or population of cells of a different endothelial cell type; and (iii) identifying the endothelial cell or population of endothelial cells as a microvascular endothelial cell or population of microvascular endothelial cells if the measured level of expression or activity is higher than would be expected in a cell or population of cells of a different endothelial cell type.

The invention additionally provides a method of identifying an endothelial cell as a microvascular endothelial cell or of identifying a population of endothelial cells as a microvascular endothelial cell population comprising steps of: (i) measuring, in an endothelial cell or in a population of endothelial cells, the level or activity of an expression product of a gene that is underexpressed in microvascular endothelial cells relative to its expression in a different endothelial cell type; (ii) comparing the level of expression or activity of the expression product to the level of expression or activity expected in a cell or population of cells of a different endothelial cell type; and (iii) identifying the endothelial cell or population of endothelial cells as a microvascular endothelial cell or population of microvascular endothelial cells if the measured level of expression or activity is lower than would be expected in a cell or population of cells of a different endothelial cell type.

This application refers to various patents, patent applications, journal articles, and other publications, all of which are incorporated herein by reference. In addition, the following standard reference works are incorporated herein by reference: Current Protocols in Molecular Biology, Current Protocols in Immunology, Current Protocols in Protein Science, and Current Protocols in Cell Biology, John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Rodd 1989 “Chemistry of Carbon Compounds”, vols. 1-5 and supps, Elsevier Science Publishers, 1989; “Organic Reactions”, vols 1-40, John Wiley and Sons, New York, NY, 1991; March 2001, “Advanced Organic Chemistry”, 5th ed. John Wiley and Sons, New York, NY.

### BRIEF DESCRIPTION OF THE DRAWING

FIG. 1A shows a heatmap presenting expression levels of the genes identified as endothelial cell specific in endothelial cell samples and non-endothelial cell samples. The heatmap shows individual hybridizations arranged along the x-axis, with relative ratios of expression (cellular RNA compared to HUVEC RNA) indicated by color. Color intensity is scaled within each row, so that the highest expression corresponds to bright yellow, and the lowest expression corresponds to bright blue.

FIG. 1B shows the separation of signals between different cell types obtained using a Gaussian error model for classification for three representative endothelial cell specific genes.

FIG. 2A shows a heatmap presenting expression levels of genes that are differentially expressed in HAEC, HCAEC, and HMVEC. Color intensity is scaled within each row so that the highest expression corresponds to bright yellow, and the lowest expression corresponds to bright blue.

FIG. 2B shows Gaussian distribution plots for six representative differentially expressed genes, showing the separation of signals between the different cell types. The letters represent actual data points. Color intensity is scaled within each row so that the highest expression corresponds to bright yellow, and the lowest expression corresponds to bright blue.

FIG. 3A shows a heatmap presenting expression levels of a portion of the genes found to be preferentially expressed in HAEC (lower panel) and HCAEC (upper panel).

FIG. 3B shows genes that were identified as being preferentially expressed at higher levels (lower panel) and lower levels (upper panel) in HMVEC compared to other endothelial cell types.

FIG. 4A shows RNA blot studies of endothelial and non-endothelial RNAs, employing radiolabeled probes for five putative endothelial cell-specific ESTs as well as two positive control genes, CD31 and multimerin.

FIG. 4B shows Gaussian distributions of normalized red/green rations derived from microarray studies investigating cell-specific expression of genes shown in FIG. 4A.

FIG. 5 shows whole mount in situ hybridization of putative endothelial cell-specific ESTs. Digoxigenin labeled cRNA probes were synthesized from cDNAs representing mouse orthologs of human sequences, and hybridized to embryonic day 9.5 mouse embryos. FIG. 5A shows whole mount in situ hybridization pattern for the gene AA4. The in situ pattern suggests expression in all endothelial cells at this stage of development, including those forming the intersomitic vessles (arrow) and those in the developing brain (arrowhead). FIG. 5B shows in situ hybridization with the mouse ortholog of human EST AW772163, revealing expression in endothelial cells of the aorta and vitelline veins (arrows), as well as cells of the developing somites (arrowhead). FIG. 5C shows in situ hybridization with the mouse ortholog of human EST W81545, showing hybridization to endothelial cells in vascular structures in the developing brain (arrow) and (FIG. 5D) yolk sac (arrows).

### DEFINITIONS

To facilitate understanding of the description of the invention, the following definitions are provided. It is to be understood that, in general, terms not otherwise defined are to be given their meaning or meanings as generally accepted in the art.

Agonist: As used herein, the term “agonist” is to be given its meaning as understood in the art and generally refers to a molecule that increases or prolongs the duration or magnitude of the effect of a second molecule (target molecule). Agonists may include proteins, nucleic acids, carbohydrates, lipids, small molecules, ions, or any other molecules that modulate the effect of the second molecule. An agonist may be a direct agonist, in which case it is a molecule that exerts its effect by binding to the target molecule, or an indirect agonist, in which case it exerts its effect via a mechanism other than binding to the target molecule (e.g., by altering expression, stability, or activity of the target molecule by a means that does not require binding to the target, by interacting with an intermediate in a pathway involving the target, etc.)

Antagonist: As used herein, the term “antagonist” is to be given its meaning as understood in the art and generally refers to a molecule that decreases or reduces the duration or magnitude of the effect of a second molecule (target molecule). Antagonists may include proteins, nucleic acids, carbohydrates, lipids, small molecules, ions, or any other molecules that modulate the effect of the second molecule. An antagonist may be a direct antagonist, in which case it is a molecule that exerts its effect by binding to the target molecule, or an indirect antagonist, in which case it exerts its effect via a mechanism other than binding to the target molecule (e.g., by altering expression, stability, or activity of the target molecule by a means that does not require binding to the target, by interacting with an intermediate in a pathway involving the target, etc.)

Antibody: In general, the term “antibody” refers to an immunoglobulin, which may be natural or wholly or partially synthetically produced in various embodiments of the invention. An antibody may be derived from natural sources (e.g., purified from a rodent, rabbit, chicken (or egg) from an animal that has been immunized with an antigen or a construct that encodes the antigen) partly or wholly synthetically produced. An antibody may be a member of any immunoglobulin class, including any of the human classes: IgG, IgM, IgA, IgD, and IgE. The antibody may be a fragment of an antibody such as an Fab′, F(ab′)₂, scFv (single-chain variable) or other fragment that retains an antigen binding site, or a recombinantly produced scFv fragment, including recombinantly produced fragments. See, e.g., Allen, T., Nature Reviews Cancer, Vol.2, 750-765, 2002, and references therein. Preferred antibodies, antibody fragments, and/or protein domains comprising an antigen binding site may be generated and/or selected in vitro, e.g., using techniques such as phage display (Winter, G. et al.1994. Annu. Rev. Immunol. 12:433-455, 1994), ribosome display (Hanes, J., and Pluckthun, A. Proc. Natl. Acad. Sci. USA. 94:4937-4942, 1997), etc. In various embodiments of the invention the antibody is a “humanized” antibody in which for example, a variable domain of rodent origin is fused to a constant domain of human origin, thus retaining the specificity of the rodent antibody. It is noted that the domain of human origin need not originate directly from a human in the sense that it is first synthesized in a human being. Instead, “human” domains may be generated in rodents whose genome incorporates human immunoglobulin genes. See, e.g., Vaughan, et al., Nature Biotechnology, 16: 535-539, 1998. An antibody may be polyclonal or monoclonal, though for purposes of the present invention monoclonal antibodies are generally preferred.

Cancer: Cancer refers to a malignant tumor (e.g., lung cancer) or growth of cells (e.g., leukemia). Cancers tend to be less differentiated than benign tumors, grow more rapidly, show infiltration, invasion and destruction, and may metastasize. Cancers include, but are not limited to, fibrosarcoma, myxosarcoma, angiosarcoma, leukemia, squamous cell carcinoma, basal cell carcinoma, malignant melanoma, renal cell carcinoma, hepatocellular carcinoma, etc.

Diagnostic information: As used herein, diagnostic information or information for use in diagnosis is any information that is useful in determining whether a subject has or is susceptible to developing a disease or condition and/or in classifying the disease or condition into a phenotypic category or any category having significance with regards to the prognosis of or likely response to treatment of the disease or condition. The term includes prenatal diagnosis, i.e., diagnosis performed prior to the birth of the subject, including performing genetic testing on germ cells (ova and/or sperm). The term also includes determining the genotype of a subject with respect to a VECSM gene for any purpose.

Differential expression: A gene or cDNA clone exhibits differential expression at the RNA level if its RNA transcript varies in abundance between different cell types, tissues, samples, etc., or at different times. A gene exhibits differential expression at the protein level if a polypeptide encoded by the gene or cDNA clone varies in abundance between different cell types, tissues, samples, etc., or at different times. In the context of a microarray experiment, differential expression generally refers to differential expression at the RNA level. “Differential expression”, as used herein, refers to both quantitative as well as qualitative differences in the temporal and/or tissue expression patterns. In general, differentially expressed genes may be used to identify or detect particular cell types, tissues, physiological states, etc., to distinguish between different cell types, tissues, or physiological states. Detection and/or measurement of the expression of such genes (referred to herein as “indicator genes”) may be useful, for example, in providing diagnostic or prognostic information related to a disease or clinical condition, for monitoring the physiological state of a cell, tissue, or organism (including monitoring the response to therapy or the progression of disease). Such genes may also be referred to herein as “diagnostic target genes” or “diagnostic targets”. Expression products of these genes (RNA or polypeptide) may also be referred to as diagnostic targets or indicators. In certain embodiments of the invention a gene may be considered differentially expressed if its expression product exhibits differences in form (e.g., phosphorylation state) between different cell type.

In addition, certain genes that are differentially expressed in cells, tissues, etc., represent “therapeutic targets” or “therapeutic target genes”, in that modulating (e.g., increasing, decreasing, or altering temporal properties) expression of such genes and/or modulating the activity of their encoded polypeptides, may alter the biochemical or physiological properties of the cell or tissue so as to treat or prevent a disease or clinical condition. For example, in the context of the present invention, modulation of the expression of certain of the differentially expressed genes described herein may treat or prevent a disease or clinical condition associated with inappropriate or excessive angiogenesis, inadequate blood supply, etc. Expression products (RNA or polypeptide) of the therapeutic target genes may also be referred to as therapeutic targets.

Certain preferred therapeutic targets include, but are not limited to, genes whose encoded polypeptide comprises an extracellular portion. The prediction of protein orientation with respect to the cell membrane and the existence of transmembrane domains can be performed using the program TMpred (K. Hofmann & W. Stoffel (1993) TMbase—A database of membrane spanning proteins segments. Biol. Chem. Hoppe-Seyler 347,166) and/or the methods described in Erik L. L. Sonnhammer, Gunnar von Heijne, and Anders Krogh: A hidden Markov model for predicting transmembrane helices in protein sequences. In Proc. of Sixth Int. Conf on Intelligent Systems for Molecular Biology, p 175-182 Ed J. Glasgow, T. Littlejohn, F. Major, R. Lathrop, D. Sankoff, and C. Sensen. Menlo Park, Calif.: AAAI Press, 1998.

Certain of the differentially expressed genes (which may be referred to herein as “interacting genes”), while not preferred therapeutic targets themselves, may interact with other genes (e.g., in a biochemical pathway) that are preferred targets for the treatment or prevention of a disease or clinical condition. It will be evident that interacting genes can also be indicator genes, and target genes may also be indicator genes and/or interacting genes.

Effective amount: In general, the “effective amount” of an active agent refers to the amount necessary to elicit a desired biological response. As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses. For example, in the case of anti-neoplastic agents, the effective amount may be the amount of agent needed to reduce the size of the primary tumor, to reduce the size of a secondary tumor, to reduce the number of metastases, to reduce the growth rate of a tumor, to reduce the ability of the primary tumor to metastasize, to increase life expectancy, etc.

Endothelial cell: The term “endothelial cell” is to be given its meaning as generally accepted in the art, i.e., the innermost layer of cells that line the cavities of the heart, blood vessels (including capillaries), and lymph vessels. The terms “endothelium” and “vascular endothelium” are used interchangeably herein.

Gene: For the purposes of the present invention, the term “gene” has its meaning as understood in the art. In general, a gene is taken to include gene regulatory sequences (e.g., promoters, enhancers, etc.) and/or intron sequences, in addition to coding sequences (open reading frames). It will further be appreciated that definitions of “gene” include references to nucleic acids that do not encode proteins but rather encode functional RNA molecules such as tRNAs. For the purpose of clarity it is noted that, as used in the present application, the term “gene” generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid.

Gene product or expression product: A “gene product” or “expression product” is, in general, an RNA transcribed from the gene (e.g., either pre- or post-processing) or a polypeptide encoded by an RNA transcribed from the gene (e.g., either pre- or post-modification).

Hybridize: The term “hybridize”, as used herein, refers to the interaction between two complementary nucleic acid sequences. The phrase “hybridizes under high stringency conditions” describes an interaction that is sufficiently stable that it is maintained under art-recognized high stringency conditions. Guidance for performing hybridization reactions can be found, for example, in Current Protocols in Molecular Biology, John Wiley & Sons, N.Y., 6.3.1-6.3.6, 1989, and more recent updated editions, all of which are incorporated by reference. See also Sambrook, Russell, and Sambrook, Molecular Cloning: A Laboratory Manual, 3^{rd} ed., Cold Spring Harbor Laboratory Press, Cold Spring Harbor, 2001. Aqueous and nonaqueous methods are described in that reference and either can be used. Typically, for nucleic acid sequences over approximately 50-100 nucleotides in length, various levels of stringency are defined, such as low stringency (e.g., 6× sodium chloride/sodium citrate (SSC) at about 45° C., followed by two washes in 0.2×SSC, 0.1% SDS at least at 50° C. (the temperature of the washes can be increased to 55° C. for medium-low stringency conditions)); medium stringency (e.g., 6×SSC at about 45° C., followed by one or more washes in 0.2×SSC, 0.1% SDS at 60° C.); high stringency (e.g., 6×SSC at about 45° C., followed by one or more washes in 0.2×SSC, 0.1% SDS at 65° C.); and very high stringency (e.g., 0.5M sodium phosphate, 0.1% SDS at 65° C., followed by one or more washes at 0.2×SSC, 1% SDS at 65° C.) Hybridization under high stringency conditions only occurs between sequences with a very high degree of complementarity. One of ordinary skill in the art will recognize that the parameters for different degrees of stringency will generally differ based various factors such as the length of the hybridizing sequences, whether they contain RNA or DNA, etc. For example, appropriate temperatures for high, medium, or low stringency hybridization will generally be lower for shorter sequences such as oligonucleotides than for longer sequences.

Isolated: As used herein, “isolated” means 1) separated from at least some of the components with which it is usually associated in nature; 2) prepared or purified by a process that involves the hand of man; and/or 3) not occurring in nature.

Ligand: As used herein, “ligand” means a molecule that specifically binds to a target such as a polypeptide through a mechanism other than an antigen-antibody interaction. The term encompasses, for example, polypeptides, peptides, and small molecules, either naturally occurring or synthesized, including molecules whose structure has been invented by man. Although the term is frequently used in the context of receptors and molecules with which they interact and that typically modulate their activity, the term as used herein applies more generally.

Marker: A “marker” may be any gene or gene product (e.g., protein, peptide, mRNA) that indicates or identifies a particular diseased or physiological state (e.g., carcinoma, normal, dysplasia) or indicates or identifies a particular cell type, tissue type, or origin. The expression or lack of expression of a marker gene may indicate a particular physiological or diseased state of a patient, organ, tissue, or cell. Preferably, the expression or lack of expression may be determined using standard techniques such as Northern blotting, in situ hybridization, RT-PCR, sequencing, immunochemistry, immunoblotting, oligonucleotide or cDNA microarray or membrane array, protein microarray analysis, mass spectrometry, etc. In certain embodiments of the invention, the level of expression of a marker gene is quantifiable.

Operably linked: As used herein, “operably linked” refers to a relationship between two nucleic acid sequences wherein the expression of one of the nucleic acid sequences is controlled by, regulated by, modulated by, etc., the other nucleic acid sequence. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence is covalently linked, either directly or indirectly, to such a sequence, although any effective three-dimensional association is acceptable.

Peptide, polypeptide, or protein: According to the present invention, a “peptide”, “polypeptide”, or “protein” comprises a string of at least three amino acids linked together by peptide bonds. The terms may be used interchangeably although a peptide generally represents a string of between approximately 8 and 20 amino acids. Peptide may refer to an individual peptide or a collection of peptides. Inventive peptides preferably contain only natural amino acids, although non-natural amino acids (i.e., compounds that do not occur in nature but that can be incorporated into a polypeptide chain; see, for example, the Web site having URL www.cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide, but such modifications may confer desirable properties, e.g., enhanced biological activity, on the peptide.

Polynucleotide or oligonucleotide: “Polynucleotide” or “oligonucleotide” refers to a polymer of nucleotides. Typically, a polynucleotide comprises at least three nucleotides. The polymer may include natural nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphorarnidite linkages).

Prognostic information and predictive information: As used herein the terms “prognostic information” and “predictive information” are used interchangeably to refer to any information that may be used to foretell any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

Purified, as used herein, means separated from many other compounds or entities. A compound or entity may be partially purified, substantially purified, or pure, where it is pure when it is removed from substantially all other compounds or entities, i.e., is preferably at least about 90%, more preferably at least about 91%, 92%, 93%, 94%, 95%, 96%, 97%, 98%, 99%, or greater than 99% pure. In the context of a preparation of a single nucleic acid molecule, a preparation may be considered substantially pure if the nucleic acid represents a majority of all nucleic acid molecules in the preparation, preferably at least 75%, yet more preferably at least 90%, or greater, as listed above.

Regulatory sequence: The term “regulatory sequence” is used herein to describe a region of nucleic acid sequence that directs, enhances, or inhibits the expression (particularly transcription, but in some cases other events such as splicing or other processing) of sequence(s) with which it is operatively linked. The term includes promoters, enhancers and other transcriptional control elements. In some embodiments of the invention, regulatory sequences may direct constitutive expression of a nucleotide sequence; in other embodiments, regulatory sequences may direct tissue-specific and/or inducible expression. For instance, non-limiting examples of tissue-specific promoters appropriate for use in mammalian cells include lymphoid-specific promoters (see, for example, Calame et al., Adv. Immunol. 43:235, 1988) such as promoters of T cell receptors (see, e.g., Winoto et al., EMBO J. 8:729, 1989) and immunoglobulins (see, for example, Banerji et al., Cell 33:729, 1983; Queen et al., Cell 33:741, 1983), and neuron-specific promoters (e.g., the neurofilament promoter; Byrne et al., Proc. Natl. Acad. Sci. USA 86:5473, 1989). Developmentally-regulated promoters are also encompassed, including, for example, the murine hox promoters (Kessel et al., Science 249:374, 1990) and the α-fetoprotein promoter (Campes et al., Genes Dev. 3:537, 1989). In some embodiments of the invention regulatory sequences may direct expression of a nucleotide sequence only in cells that have been infected with an infectious agent. For example, the regulatory sequence may comprise a promoter and/or enhancer such as a virus-specific promoter or enhancer that is recognized by a viral protein, e.g., a viral polymerase, transcription factor, etc.

Sample: As used herein, a “sample” obtained from a subject may include, but is not limited to, any or all of the following: a cell or cells, a portion of tissue, blood, serum, ascites, urine, saliva, amniotic fluid, cerebrospinal fluid, and other body fluids, secretions, or excretions. The sample may be a tissue sample obtained, for example, from skin, muscle, buccal or conjunctival mucosa, placenta, gastrointestinal tract or other organs. A sample of DNA from fetal or embryonic cells or tissue can be obtained by appropriate methods, such as by amniocentesis or chorionic villus sampling. The term “sample” may also refer to any material derived by isolating, purifying, and/or processing a sample obtained directly from a subject. Derived samples may include nucleic acids or proteins extracted from the sample or obtained by subjecting the sample to techniques such as amplification or reverse transcription of mRNA, etc. A derived sample may be, for example, a homogenate, lysate, or extract prepared from a tissue, cells, or other constituent of an organism (e.g., a body fluid).

Short interfering RNA: The term short interfering RNA (siRNA) refers to an RNA duplex that is approximately 19 basepairs long and optionally further comprises one or two single-stranded overhangs. An siRNA may be formed from two RNA molecules that hybridize together, or may alternatively be generated from a single RNA molecule that includes a self-hybridizing portion. According to certain embodiments of the invention free 5′ ends of siRNA molecules have phosphate groups, and free 3′ ends have hydroxyl groups while according to other embodiments free 5′ ends lack phosphate groups and/or free 3′ ends lack hydroxyl groups. The duplex portion of an siRNA may, but typically does not, contain one or more bulges consisting of one or more unpaired nucleotides. One strand of an siRNA includes a portion that hybridizes with a target transcript. In certain preferred embodiments of the invention, one strand of the siRNA is precisely complementary with a region of the target transcript, meaning that the siRNA hybridizes to the target transcript without a single mismatch. In other embodiments of the invention one or more mismatches between the siRNA and the targeted portion of the target transcript may exist. In most embodiments of the invention in which perfect complementarity is not achieved, it is generally preferred that any mismatches be located at or near the siRNA termini.

Short hairpin RNA: The term short hairpin RNA (shRNA) refers to an RNA molecule comprising at least two complementary portions hybridized or capable of hybridizing to form a double-stranded (duplex) structure sufficiently long to mediate RNAi (typically at least 19 base pairs in length), and at least one single-stranded portion, typically between approximately 1 and 10 nucleotides in length that forms a loop. The duplex portion may, but typically does not, contain one or more bulges consisting of one or more unpaired nucleotides. As described further below, shRNAs are thought to be processed into siRNAs by the conserved cellular RNAi machinery. Thus shRNAs are precursors of siRNAs and are, in general, similarly capable of inhibiting expression of a target transcript.

Small molecule: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

Specific binding: As used herein, the term “specific binding” refers to an interaction between a target molecule (typically a target polypeptide) and a binding molecule such as an antibody or ligand. The interaction is typically dependent upon the presence of a particular structural feature of the target molecule such as an antigenic determinant or epitope recognized by the binding molecule. For example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute but generally refers to the context in which the binding is performed. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies having a sufficient degree of specificity to perform appropriately in any given application (e.g., for detection of a target molecule, for therapeutic purposes, etc). It is also to be understood that specificity may be evaluated in the context of additional factors such as the affinity of the binding molecule for the target polypeptide versus the affinity of the binding molecule for other targets, e.g., competitors. If a binding molecule exhibits a high affinity for a target molecule that it is desired to detect and low affinity for nontarget molecules, the antibody will likely be an acceptable reagent for immunodiagnostic purposes. Once the specificity of a binding molecule is established in one or more contexts, it may be employed in other, preferably similar, contexts without necessarily re-evaluating its specificity. In the context of an interaction between an antibody or ligand and a VECSM (vascular endothelial cell specific marker) polypeptide, according to certain embodiments of the invention a molecule exhibits specific binding if it binds to the VECSM polypeptide at least 5 times as strongly as to other polypeptides present in a cell lysate. According to certain embodiments of the invention a molecule exhibits specific binding if it binds to the VECSM polypeptide at least 10 times as strongly as to other polypeptides present in a cell lysate. According to certain embodiments of the invention a molecule exhibits specific binding if it binds to the VECSM polypeptide at least 50 times as strongly as to other polypeptides present in a cell lysate. According to certain embodiments of the invention a molecule exhibits specific binding if it binds to the VECSM polypeptide at least 100 times as strongly as to other polypeptides present in a cell lysate. The cell lysate can be an endothelial cell lysate or a non-endothelial cell lysate.

Subject: The term subject, as used herein, refers to an individual to whom an agent is to be delivered, e.g., for experimental, diagnostic, and/or therapeutic purposes. Preferred subjects are mammals, particularly domesticated mammals (e.g., dogs, cats, etc.), primates, or humans.

Targeted: An siRNA or shRNA or an siRNA or shRNA sequence, (or any other RNAi-inducing entity such as a vector whose presence within a cell results in transcription of one or more RNAs that hybridize or self-hybridize to form an siRNA, shRNA or precursor thereof) is considered to be targeted to a target transcript for the purposes described herein if 1) the stability of the target transcript is reduced in the presence of the siRNA or shRNA as compared with its absence; and/or 2) the siRNA or shRNA shows at least about 90%, more preferably at least about 91%, 92%, 93%, 94%, 95%, 96%, 97%, 98%, 99%, or 100% precise sequence complementarity with the target transcript for a stretch of at least about 15, more preferably at least about 17, yet more preferably at least about 18 or 19 to about 21-23 nucleotides; and/or 3) one strand of the siRNA or one of the self-complementary portions of the shRNA hybridizes to the target transcript under stringent conditions for hybridization of small (<50 nucleotide) RNA molecules in vitro and/or under conditions typically found within the cytoplasm or nucleus of mammalian cells. An RNA-inducing vector whose presence within a cell results in production of an siRNA or shRNA that is targeted to a transcript is also considered to be targeted to the target transcript. Since the effect of targeting a transcript is to reduce or inhibit expression of the gene that directs synthesis of the transcript, an siRNA or shRNA targeted to a transcript is also considered to target the gene that directs synthesis of the transcript even though the gene itself (i.e., genomic DNA) is not thought to interact with the siRNA, shRNA, or components of the cellular silencing machinery. Thus as used herein, an siRNA, shRNA, or RNAi-inducing vector that targets a transcript is understood to target the gene that provides a template for synthesis of the transcript.

Treating: As used herein, “treating” includes reversing, alleviating, inhibiting the progress of, preventing, or reducing the likelihood of the disease, disorder, or condition to which such term applies, or one or more symptoms or manifestations of such disease, disorder or condition.

Tumor: As used in the present application, “tumor” refers to an abnormal growth of cells. The growth of the cells of a tumor typically exceed the growth of normal tissue and tends to be uncoordinated. The tumor may be benign (e.g., lipoma, fibroma, myxoma, lymphangioma, meningioma, nevus, adenoma, leiomyoma, mature teratoma, etc.) or malignant, i.e., cancerous (e.g., malignant melanoma, ovarian cancer, carcinoma in situ, carcinoma, adenocarcinoma, liposarcoma, mesothelioma, squamous cell carcinoma, basal cell carcinoma, colon cancer, lung cancer, etc.).

Vector: The term “vector” is used herein to refer to a nucleic acid molecule capable of mediating entry of, e.g., transferring, transporting, etc., another nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids (which may comprise sequences derived from viruses), cosmids, and virus vectors. Virus vectors include, e.g., replication defective retroviruses, adenoviruses, adeno-associated viruses, and lentiviruses. As will be evident to one of ordinary skill in the art, virus vectors may include various viral components in addition to nucleic acid(s) that mediate entry of the transferred nucleic acid.

It is also noted that where compositions and methods described in the text refer to VECSM polynucleotides, polypeptides, antibodies, siRNAs, etc.), the compositions and methods may be restricted to any one or more of these entities, or to any group thereof, including the groups identified as Group II (not previously identified as endothelial-cell specific) or III (not previously characterized).

### DETAILED DESCRIPTION OF CERTAIN PREFERRED EMBODIMENTS OF THE INVENTION

I. Overview

Vascular endothelial cells maintain the interface between the systemic circulation and soft tissues and mediate critical processes such as inflammation in a vascular bed specific fashion. Endothelial cells initiate angiogenesis and, together with smooth muscle cells, form new blood vessels. A number of diseases and clinical conditions may be associated with inappropriate or excessive vascular endothelial growth. These include, but are not limited to, cancer, atherosclerosis, restenosis, psoriasis, rheumatoid arthritis, diabetic retinopathy, endometriosis, menorrhagia, hemangiomas, and vascular malformations. Conversely, a number of diseases and clinical conditions may be associated with a reduced or inadequate blood supply, e.g., a blood supply that is insufficient to permit the affected tissue, organ, etc., to effectively perform its biological function and/or maintain its normal structure and characteristics. These include ischemic cardiovascular diseases, diabetes, peripheral arterial disease, ischemic stroke, etc.

As mentioned above, angiogenesis is essential for tumor progression and metastasis in many tumor types. Reduced or inadequate blood supply likewise plays a role in various pathologic states such as ischemic cardiovascular disease, and inducing formation of new blood vessels offers a means of treating such conditions. Scar tissue is characterized by a lack of blood vessels, and efforts to promote tissue repair and to achieve wound healing with a minimum amount of scar formation may be enhanced by therapies that increase angiogenesis. Assessment of angiogenic activities in vivo by imaging technologies is important for understanding molecular mechanisms of angiogenesis, for diagnosing diseases and following their course over time, and for monitoring the effects of angiogenic modifying agents. See, e.g., Neeman, M., “Functional and molecular MR imaging of angiogenesis: Seeing the target, seeing it work”, J Cell Biochem Suppl 2002;39:11-17.

The inventors have recognized that progress in the areas of vascular imaging, diagnosis, and development of therapeutic agents to modulate angiogenesis and other functions of vascular tissue would be greatly facilitated by knowledge of markers (e.g., genes and their encoded polypeptides), that are differentially expressed in endothelial cells and whose expression level may be used to distinguish endothelial cells from other cell types and/or to distinguish between endothelial cells present in different vascular beds. Accordingly, the present invention is based on the inventors' identification of genes whose expression occurs selectively or specifically in vascular endothelial cells, i.e., genes that are differentially expressed in endothelial cells relative to other cell types and their further discovery that certain of these genes are differentially expressed in endothelial cells present in certain vascular beds relative to their expression in endothelial cells present in other vascular beds.

As described in more detail in the Examples, the inventors applied a combination of database mining and subtraction suppression hybridization to identify a set a candidate endothelial cell specific genes. Public databases were queried to identify potential candidate genes, which were then evaluated by application of newly developed scoring metrics, resulting in a set of 384 candidate genes. Subtraction suppression hybridization was performed on endothelial cells stimulated with TGF-β, a known modulator of angiogenic vascular formation (Pepper, 1997), thus identifying an additional 288 candidates. High throughput transcriptional profiling using microarrays was then employed to select a subset of these genes that showed differential expression between endothelial cells and non-endothelial cells. Clones identified through the two complementary approaches were printed on glass slides to generate DNA microarrays. RNA samples isolated from endothelial cells originating from different vascular beds or from a variety of non-endothelial cell types were hybridized to the microarrays, and expression data were evaluated using several statistical methods.

The set of genes thus identified showed a relative expression ranging from 3-fold to 55-fold between endothelial and non-endothelial cell types. In other words, the level of expression of the genes was between 3 and 55 fold greater in endothelial cells than in other cell types tested. These genes, and their associated polypeptides and polynucleotides, have been named VECSM genes, VECSM polynucleotides, and VECSM polypeptides, where VECSM stands for “vascular endothelial cell specific marker”. When discussing these genes, the nomenclature VECSM will be taken to represent any of the vascular endothelial cell specific markers. For example, VECSM may represent VECSM4, VECSM11, etc. The VECSM genes include a subset of previously known endothelial cell specific genes (Group I), a subset known to be expressed in other tissue types but not previously known to be expressed or differentially expressed in endothelial cells (Group II), and a subset of genes that had previously been uncharacterized or minimally characterized (Group III).

The identification of known endothelial cell markers (Group I) validates the approach adopted herein, while the discovery that genes listed in Groups II and III are differentially expressed in endothelial cells greatly expands the repertoire of endothelial cell specific markers. Identification of these markers provides a wide variety reagents and methods, as described below. For example, identification of VECSM genes permits the selective targeting of molecules to endothelial cells, e.g., for purposes including, but not limited to, diagnosis, prognosis, treatment, imaging, or assessment of treatments for conditions involving the vascular endothelium. Such conditions include conditions associated with aberrant or absent endothelial growth as well as conditions in which vascular endothelium contributes to disease, as in the case of tumors that require an adequate blood supply.

Evaluation of the level of expression of these markers permits the identification of endothelial cells in tissue samples, as well as their quantitation. The expression products of the genes are also targets for diagnostic agents such as imaging molecules. These markers may also be used to selectively target active agents (e.g., therapeutic agents) to endothelial cells. In addition, the genes and their expression products (RNA and/or protein) are potential targets for therapies aimed at increasing, decreasing, altering, or otherwise modulating the activity or function of vascular tissue and/or the degree of angiogenic activity. It is noted that endothelial cells and hematopoietic cells originate from the same embryonic precursors, i.e., hemangioblasts (Suda, T., et al., Int. J. Hematol., 71(2): 99-107, 2000). Therefore the invention encompasses the use of any of the markers described herein for purposes of identifying, targeting, imaging, etc., of hematopoietic cells in addition to endothelial cells.

II. VECSM Genes, Polynucleotides, Polypeptides, and Related Reagents

A. VECSM Genes, Clones, and Polynucleotides

Genes in Groups I, II, and III are listed in Tables 1, 2, and 3, respectively. The tables list the GenBank accession number of the cDNA clone, one strand of which (i.e., the strand complementary to mRNA) was printed on the microarrays used to identify the gene, gene names, alternate name(s), and symbol(s) for the gene represented by the clone (where such name(s) or symbol(s) exist,

TABLE 1
Genes Previously Identified as Differentially Expressed in Endothelial Cells

| | | Gene Symbol/ | Fold | SAM | SAM | GER | GER | TNoM |
|---|---|---|---|---|---|---|---|---|
| Acc. No. | Gene Name | Other name | Difference | Score | Expect | Score | Rand | Score |
| R22412 | platelet/endothelial cell adhesion molecule (CD31 | PE-CAM1, CD31 | 55.5 | 6.91 | 1.25 | 0.102 | 20 | 3 |
| | antigen) | | | | | | | |
| AA423867 | multimerin | MMRN, ECM | 48.3 | 6.16 | 1.10 | 0.191 | 45 | 7 |
| W46577 | endothelial cell-specific molecule 1 | ESM1 | 27.6 | 5.85 | 1.02 | 0.130 | 27 | 3 |
| H02884 | cadherin 5, type 2, VE-cadherin (vascular | CDH5, | 23.5 | 6.64 | 1.20 | 0.046 | 3 | 1 |
| | epithelium) | VE-CADHERIN | | | | | | |
| N93476 | endothelial differentiation, sphingolipid G-protein- | EDG1 | 10.4 | 5.67 | 1.00 | 0.080 | 13 | 0 |
| | coupled recept, 1 | | | | | | | |
| AA485883 | von Willebrand factor | VWF | 10.0 | 5.88 | 1.03 | 0.065 | 10 | 1 |
| H11003 | endothelin 1 | ET1 | 6.5 | 4.62 | 0.87 | 0.129 | 26 | 4 |
| AA026831 | kinase insert domain receptor (a type III receptor | KDR, FLK1, | 5.5 | 3.44 | 0.75 | 0.331 | 96 | 9 |
| | tyrosine kinase) | VEGFR, | | | | | | |
| | | VEGFR2, | | | | | | |
| | | VEGFR-2, | | | | | | |
| | | FLK-1 | | | | | | |
| T47442 | protein C receptor, endothelial (EPCR) | EPCR | 5.0 | 3.83 | 0.79 | 0.301 | 84 | 6 |
| AA598794 | connective tissue growth factor | CTGF, IGFBP8 | 4.2 | 3.31 | 0.74 | 0.320 | 91 | 11 |
| AA150505 | complement component 1, q subcomponent, | C1QR1 | 29.1 | 6.48 | 1.13 | 0.130 | 28 | 4 |
| | receptor 1 | | | | | | | |
| AA156022 | roundabout homolog 4, magic roundabout | ROBO4 | 14.4 | 5.99 | 1.08 | 0.113 | 22 | 0 |
| | (Drosophila) | | | | | | | |
| AI888888 | melanoma cell adhesion molecule | MCAM, MUC18 | 11.6 | 5.10 | 0.94 | 0.111 | 21 | 2 |
| N54794 | serine (or cysteine) proteinase inhibitor, clade E | PAI1 | 3.8 | 3.18 | 0.71 | 0.220 | 54 | 4 |
| | (nexin, PAI1) | | | | | | | |

TABLE 2
Genes Not Previously Identified as Differentially Expressed in Endothelial Cells

| | | Gene | | | | | | |
|---|---|---|---|---|---|---|---|---|
| | | Symbol/ | | | | | | |
| | | Other | Fold | SAM | SAM | GER | GER | TNoM |
| Acc. No. | Gene Name | name | Difference | Score | Expect | Score | Rank | Score |
| AA155913 | matrix Gla protein | MGP, MGLAP | 5.6 | 3.23 | 0.72 | NA | NA | NA |
| AA424833 | bone morphogenetic protein 6 | BMP6 | 6.5 | 4.80 | 0.90 | 0.121 | 25 | 2 |
| AA875933 | EGF-containing fibulin-like extracellular matrix protein 1 | EFEMP1, FBNL | 25.0 | 5.93 | 1.04 | 0.098 | 18 | 3 |
| AA428201 | EGF-TM7-latrophilin-related protein | ETL | 12.8 | 4.91 | 0.91 | 0.193 | 46 | 5 |
| AI056548 | hedgehog interacting protein | HHIP, HIP | 10.5 | 5.16 | 0.95 | 0.181 | 42 | 3 |
| AI261621 | phospholipid scramblase 4 | PLSCR4 | 9.8 | 4.50 | 0.84 | 0.091 | 17 | 3 |
| N64508 | podocalyxin-like | PODXL, PCLP | 8.2 | 3.80 | 0.79 | 0.398 | 145 | 7 |
| AI279830 | protein phosphatase 1, regulatory (inhibitor) subunit | PPP1R16B | 6.9 | 4.54 | 0.84 | 0.063 | 9 | 2 |
| | 16B | | | | | | | |
| AA418728 | palmdelphin | PALMD, PALML | 6.3 | 4.79 | 0.89 | 0.091 | 16 | 1 |
| AA010753 | ras homolog gene family, member J (TC10-LIKE | ARHJ, TCL | 6.2 | 4.44 | 0.83 | 0.168 | 39 | 6 |
| | PROTEIN) | | | | | | | |
| H22922 | manic fringe homolog (Drosophila) | MFNG | 6.2 | 3.57 | 0.77 | 0.316 | 88 | 6 |
| AF064493 | LIM domain binding 2 | LDB2, CLIM1 | 5.9 | 3.41 | 0.74 | 0.367 | 120 | 12 |
| W68141 | protein kinase, cAMP-dependent, catalytic, alpha | PRKACA | 5.2 | 4.86 | 0.90 | 0.044 | 2 | 0 |
| AF297711 | netrin 4 | NTN4 | 5.1 | 5.18 | 0.96 | 0.062 | 8 | 2 |
| R09728 | serum deprivation response (phosphatidylserine | SDPR | 4.9 | 3.91 | 0.80 | 0.247 | 67 | 6 |
| | binding protein) | | | | | | | |
| Z99289 | laminin, alpha 4 | LAMA4 | 4.9 | 3.47 | 0.75 | 0.222 | 55 | 9 |
| AF092132 | Hs.p21-ACTIVATED KINASE 2 mRNA, complete cds | PAK2 | 4.5 | 3.19 | 0.72 | NA | NA | NA |
| AA669136 | transcription factor 4 (IMMUNOGLOBULIN | TCF4, ITF2, SEF2-1B, | 3.7 | 3.56 | 0.76 | 0.245 | 64 | 6 |
| | TRANSCRIPTION FACTOR 2) | SEF2, E2-2 | | | | | | |
| D67029 | SEC14-like 1 (S. cerevisiae) | SEC14L1 | 2.9 | 3.69 | 0.78 | 0.354 | 110 | 9 |
| B001631 | thymosin, beta 4, X chrm. (PROTHYMOSIN BETA- | TMSB4X, TMSB4, | 2.6 | 3.31 | 0.73 | 0.121 | 24 | 4 |
| | 4) | PTMB4 | | | | | | |

TABLE 3

| | | Fold | SAM | SAM | GER | GER | TNoM |
|---|---|---|---|---|---|---|---|
| Acc. No. | Gene Name | Difference | Score | Expect | Score | Rank | Score |
| AA410298 | ESTs | 29.7 | 7.33 | 1.43 | 0.027 | 1 | 0 |
| AA256482 | Hs. mRNA for FLJ00138 protein | 28.6 | 7.21 | 1.31 | 0.052 | 6 | 3 |
| AW770514 | ESTs, Highly similar to AF161403 1 HSPC285 | 20.4 | 6.51 | 1.16 | 0.067 | 11 | 1 |
| | [H. sapiens] | | | | | | |
| AI422298 | Hs., similar to hypothetical protein FLJ11110 | 11.9 | 5.43 | 0.99 | 0.145 | 35 | 4 |
| N75004 | ESTs | 11.2 | 4.91 | 0.92 | 0.170 | 40 | 5 |
| N54398 | Hs. cDNA FLJ32566 fis, clone SPLEN2000047 | 10.9 | 5.24 | 0.97 | 0.101 | 19 | 4 |
| W81545 | EST | 10.6 | 5.04 | 0.93 | 0.148 | 36 | 1 |
| R21535 | Hs. cDNA FLJ11724 fis, clone HEMBA1005331 | 9.0 | 4.05 | 0.81 | 0.290 | 79 | 5 |
| AA004368 | hypothetical protein FLJ21269 | 7.0 | 4.68 | 0.88 | 0.217 | 53 | 2 |
| AA147552 | ESTs | 5.8 | 3.42 | 0.75 | 0.133 | 31 | 8 |
| AA460732 | DKFZP564D0764 protein | 5.5 | 4.38 | 0.83 | 0.140 | 34 | 4 |
| AA418988 | Hs. mRNA; cDNA DKFZp586L0120 (from clone | 5.0 | 3.89 | 0.80 | 0.136 | 33 | 3 |
| | DKFZp586L0120) | | | | | | |
| AW772163 | hypothetical protein FLJ20401 | 4.4 | 3.76 | 0.78 | 0.258 | 72 | 6 |
| N66734 | Hs. mRNA; cDNA DKFZp586N0121 (from clone | 4.1 | 3.63 | 0.77 | 0.226 | 58 | 7 |
| | DKFZp586N0121) | | | | | | |
| AI299356 | Hs. cDNA FLJ31414 fis, clone NT2NE2000260 | 3.6 | 3.26 | 0.72 | 0.250 | 68 | 8 |
| AA668698 | ESTs | 3.4 | 3.54 | 0.76 | 0.212 | 52 | 3 |

and relevant data demonstrating differential expresion. In general, the term “VECSM Group II polynucleotide” refers to a naturally occurring polynucleotide comprising a polynucleotide having a Genbank accession number listed in Table 2, and the term “VECSM Group II polypeptide” refers to a naturally occurring polypeptide that is encoded by a VECSM Group II polynucleotide. In general, the term “VECSM Group III polynucleotide” refers to a naturally occurring polynucleotide comprising a polynucleotide having a Genbank accession number listed in Table 2, and the term “VECSM Group III polypeptide” refers to a naturally occurring polypeptide that is encoded by a VECSM Group III polynucleotide. It will be appreciated that the accession numbers listed in Tables 2 and 3 identify particular human genes, i.e., the polynucleotides whose sequence corresponds to a particular accession number are transcription products of particular human genes. The terms “VECSM Group II polypeptide” or “VECSM Group II polynucleotide”, are intended to include all naturally occurring RNA or protein expression products, respectively, of the genes identified by the accession numbers listed in Table 2. The terms “VECSM Group III polypeptide” or “VECSM Group III polynucleotide” are intended to include all naturally include all naturally occurring RNA or protein expression products, respectively, of the genes identified by the accession numbers listed in Table 2. The terms “VECSM Group III polypeptide” or “VECSM Group III polynucleotide” are intended to include all naturally occurring RNA or protein expression products, respectively, of the genes identified by the accession numbers listed in Table 3. An RNA expression product of a gene is an RNA that is transcribed from the gene. A protein expression product of a gene is a polypeptide that is translated from an RNA that is transcribed from the gene, including polypeptides translated from RNAs that have been subjected to co-transcriptional or post-transcriptional modification such as splicing, trans-splicing, editing, etc. The polypeptides include polypeptides that have been subjected to co-translational or post-translational processing such as cleavage, provided that at least 8 contiguous amino acids of the original polypeptide remain intact. It is noted that although accession numbers may change, the current accession number corresponding to any particular previously used accession number is readily available and accessible to one of ordinary skill in the art.

Any of the Genbank accession numbers listed in Table 2 or 3 may be used by one of ordinary skill in the art to identify additional polynucleotide sequences from the corresponding gene using publicly available databases. For example, by entering an accession number for a first polynucleotide into the Unigene database (available at the web site having URL www.ncbi.nlm.nih.gov) one of ordinary skill in the art can obtain a Unigene ID number. This number refers to a set of cDNA clones all of which correspond to the same gene (i.e., they have the sequence of an mRNA transcribed from that gene). The Unigene number can then be used by one of ordinary skill in the art to obtain a list of accession numbers, each of which identifies a cDNA clone representing an mRNA that is transcribed from the same gene as the first polynucleotide. The database further includes the sequences of the polynucleotides. For example, the first clone listed in Table 3 has the accession number AA410298. Appendix A, page 1, shows use of this accession number to obtain a Unigene ID number as of Mar. 17, 2004. Appendix A, page 3 shows the Unigene ID number (Hs. 483538) obtained by searching with accession number AA410298. This ID number is linked to a list of 42 cDNA clones, identified by accession number, all of which represent the same gene. Appendix A, pages 4-7, shows this list. Such polynucleotides, corresponding to the same genes as the clones identified by accession number in Tables 2 and 3, are encompassed within the terms “VECSM Group II polynucleotide” and “VECSM Group III polynucleotide”. It will be appreciated that the list of clones is likely to grow as more data is deposited in the public databases and additional clones with areas of overlapping sequence are identified.

As used herein, consistently with its usage in the art, the term “cDNA” generally refers to a double-stranded DNA, one strand of which is complementary to mRNA. The term may also refer to a single DNA strand obtained by reverse transcribing mRNA or to the complement of such a strand. In general, the sequence of a cDNA clone as used or provided herein is the sequence of the strand whose sequence is identical to that of part or all of the mRNA from which the first strand of the cDNA was transcribed (except that the cDNA contains Ts instead of Us). As used herein, a gene, cDNA, or mRNA “corresponds to” or “is represented by” a cDNA clone if the gene, the cDNA, or the mRNA sequence includes at least the coding portion of the sequence of the cDNA clone and, optionally, all or part of any untranslated 5′ or 3′ region. Note that in the case of a gene, the sequence may be interrupted by introns. In addition, the sequence of the gene will generally not include elements such as the polyA tail that are added to the mRNA following transcription. Note also that in any particular individual, the sequence of the gene, cDNA, and/or mRNA that corresponds to the clone may differ by one or more nucleotides from that of the clone due, for example, to polymorphisms or mutations. In particular, certain diseases may be caused by mutations in the genes identified herein. By “full length cDNA” corresponding to a clone is meant the complement of naturally occurring full length mRNA (e.g., mRNA that has not been subject to degradation) that contains a region of complementarity to the clone, where the region of complementarity is typically at least 20 nucleotides in length and is generally at least 50 nucleotides in length. Full length mRNA is understood to mean the RNA transcript, and may or may not include changes due to any post-transcriptional modification such as addition of a polyA tail.

It is noted that although in some cases the clones represent less than full length cDNAs, their length is sufficient to allow identification of the corresponding gene and full length cDNA that includes the clone's sequence as part of its sequence (although such identification is not required in order to practice many aspects of the invention). Thus based solely on the information available from the sequence of the clones, the present invention enables the identification of the full length cDNA and its complement, antisense, ribozyme, and RNAi-inducing molecules targeted to the corresponding mRNA transcript, etc., and methods of using these reagents. In addition, the sequence of the clones enables the identification of the full length polypeptide encoded by the gene, antibodies to the polypeptide, and related reagents and methods of use thereof. These and other aspects of the invention are described more fully below.

For purposes of description, the terms “VECSM clone” will be taken to mean the cDNA clone, one strand of which was printed on the microarrays that were used to identify genes that are differentially expressed in endothelial cells. The term “VECSM gene” refers to a region of genomic DNA from which mRNA whose sequence comprises the sequence of the VECSM clone (except that the mRNA contains Us rather than Ts) is transcribed, having the properties of a gene as defined above. As mentioned above, the gene may be interrupted by introns, thus the portion of the VECSM genomic DNA comprising the VECSM clone sequence may not be contiguous.

The term “VECSM polynucleotide” will be taken to include the corresponding VECSM clone, any naturally occurring polynucleotides, either single or double stranded (other than genomic DNA) whose sequence comprises or consists of the sequence of the VECSM clone, including mRNA whose sequence comprises the sequence of the clone (which will be referred to as “VECSM mRNA”), and any polynucleotides complementary to such naturally occurring polynucleotides (e.g., a cDNA strand complementary to VECSM mRNA). cDNAs, one strand of which is complementary to VECSM mRNA will be referred to as “VECSM cDNA”.

According to certain embodiments of the invention VECSM polynucleotides also include any polynucleotide that comprises or consists of at least 60 contiguous nucleotides of any of the VECSM polynucleotides described above. VECSM polynucleotides are useful for a variety of purposes including, but not limited to, use as probes or elements on a microarray to detect expression of the corresponding gene, as templates for production of VECSM polypeptides, as therapeutic agents, and as targets for diagnostic and/or therapeutic agents, etc.

In addition to the VECSM polynucleotides described above, the invention provides polynucleotides that are able to hybridize to any of the VECSM polynucleotide sequences discussed above under various conditions of stringency, e.g., low, medium, or high stringency. In general, a hybridizing polynucleotide will have a sequence either partially or fully complementary with the polynucleotide to which it hybridizes. Hybridization conditions of various stringency are well known in the art and are, in general, governed by the concentration of reagents such as salts and formamide in the hybridization buffer as well as by the temperature at which hybridization is performed. The selection of appropriate hybridization conditions will typically be determined by the purpose for which hybridization is to be carried out and is a matter of choice for the practitioner.

B. VECSM Oligonucleotides

The invention provides VECSM oligonucleotides corresponding to each of the VECSM genes. The term “VECSM oligonucleotide” includes any polynucleotide, and its complement, that consists of between 8 and 60 contiguous nucleotides of any of the VECSM polynucleotides described above and is 60 or fewer nucleotides in length. According to certain embodiments of the invention the oligonucleotide consists of at least 8 contiguous nucleotides of any of the VECSM polynucleotides described above. According to certain embodiments of the invention the oligonucleotide consists at least 10 contiguous nucleotides of any of the VECSM polynucleotides described above. In particular embodiments the VECSM polynucleotide is a VECSM Group III polynucleotide.

The invention also provides a substantially purified oligonucleotide, and its complement, comprising a portion that specifically hybridizes to at least 8 consecutive nucleotides of a nucleic acid that encodes a VECSM Group III polypeptide, or that specifically hybridizes to at least 10 consecutive nucleotides of any of these nucleic acids. The invention further provides oligonucleotides as described above, wherein the relevant lengths are between 10 and 60, between 12 and 60, or between 15 and 60. In certain embodiments of the invention the oligonucleotide is labeled or conjugated, e.g., with a fluorescent moiety, enzyme, enzyme substrate, or radioisotope, which may facilitate detection of the oligonucleotide. According to certain embodiments of the invention, the sequence of a VECSM oligonucleotide may differ from that of the corresponding VECSM polynucleotide at not more than 10% of the nucleotide positions. According to certain embodiments of the invention, the sequence of a VECSM oligonucleotide may differ from that of the corresponding VECSM polynucleotide at not more than 20% of the nucleotide positions.

VECSM oligonucleotides have a variety of uses including, but not limited to, use as probes to detect complementary nucleic acid sequences, for detection of mRNA expression levels of the corresponding gene (e.g., in cells or tissues, tissue samples isolated from a patient, etc.), for detection of amplification or mutations in the corresponding gene or an mRNA transcript, use as PCR primers (e.g., for amplification or mutagenesis of a VECSM gene, transcript, etc.), and for purposes of reducing or inhibiting expression of a VECSM gene (see description of VECSM antisense oligonucleotides and siRNAs below). The invention encompasses methods of using VECSM oligonucleotides for these purposes. According to certain embodiments of the invention, the VECSM oligonucleotide is between approximately 15 and 30 nucleotides in length, which may be preferred for purposes of PCR amplification. Guidelines for selecting primers for PCR amplification are well known in the art. See, e.g., McPherson, M., et al., PCR Basics: From Background to Bench, Springer-Verlag, 2000. A variety of computer programs for designing primers are available, e.g., ‘Oligo’ (National Biosciences, Inc, Plymouth Minn.), MacVector (Kodak/IBI), and the GCG suite of sequence analysis programs (Genetics Computer Group, Madison, Wis. 53711).

For use in mutagenesis, it may be preferable to use oligonucleotides or polynucleotides between approximately 20 and 100 nucleotides in length. It is to be understood that where the VECSM oligonucleotides or polynucleotides are used for mutagenesis, the sequence will differ from that of the gene or portion thereof that is being mutated.

C. VECSM Microarrays

The invention further provides a microarray for detection or quantification of endothelial cells or angiogenic activity comprising one or more oligonucleotides or cDNAs that specifically hybridizes to a VECSM Group II or Group III polynucleotide. In certain embodiments of the invention the microarray comprises at least 5 or at least 10 different oligonucleotides or cDNAs, each of which specifically hybridizes to a different VECSM Group II or Group III polynucleotide. The oligonucleotides and/or microarrays of the invention may be provided as a kit, which may comprise packaging and instructions for use, a buffer, nucleotides, a polymerase, an enzyme, a positive control sample, a negative control sample, and a negative control primer or probe.

D. VECSM Polypeptides

By the term “VECSM polypeptide” is meant a polypeptide encoded by a VECSM clone, or a naturally occurring polypeptide whose sequence comprises or consists of the sequence of a polypeptide encoded by a VECSM clone. VECSM polypeptides include naturally occurring polypeptides encoded by VECSM cDNA, mRNA, or genomic DNA. VECSM polypeptides include polypeptides encoded by genes corresponding to the polynucleotides identified by accession number in Tables 2 and 3. It will be appreciated that a VECSM polynucleotide sequence can be translated using the genetic code to arrive at the amino acid sequences of the encoded VECSM polypeptide. In various embodiments of the invention VECSM polypeptides include any polypeptide having the sequence of a naturally occurring polypeptide that comprises or, in various embodiments of the invention consists of at least a consecutive 50 amino acid portion of any of the VECSM polypeptides described above. In various embodiments of the invention VECSM polypeptides include any artificially produced polypeptide comprising or, in various embodiments of the invention consisting of at least a consecutive 50 amino acid portion of any of the VECSM polypeptides described above. In certain embodiments of the invention the term VECSM polypeptide includes polypeptides encoded by any naturally occurring variants of the VECSM polynucleotides as described above, e.g., naturally occurring variants of the VECSM polypeptides described above, whether resulting from alterations in the VECSM coding sequence, posttranscriptional processing, or posttranslational modification. VECSM polypeptides are useful for a variety of purposes including, but not limited to, use to generate antibodies (uses of which are described below), as targets for diagnostic and/or therapeutic agents, etc.

The invention further provides polypeptides comprising a VECSM polypeptide that is modified to include an additional polypeptide sequence, e.g., a tag such as an epitope tag. The tag may, but need not be, be present as an N or C terminal fusion.

E. VECSM Peptides

The invention further provides VECSM peptides. By “VECSM peptide” is meant any polypeptide that consists of between 8 and 50 contiguous amino acid residues of a VECSM polypeptide. VECSM peptides have a variety of uses including, but not limited to, use as antigens for the generation of antibodies.

The invention further provides polypeptides comprising a VECSM polypeptide that is modified to include an additional polypeptide sequence, e.g., a tag such as an epitope tag. The tag may, but need not be, be present as an N or C terminal fusion.

F. VECSM Polynucleotide Variants

The invention further provides a number of different types of VECSM polynucleotide variants. In particular, the invention provides VECSM polynucleotide variants in which one or more nucleotides is replaced with a different nucleotide that does not result in a change in the encoded polypeptide, i.e., a variant in which one or more codons is altered to a different codon that codes for the same amino acid. As is well known in the art, due to the degeneracy of the genetic code the same polypeptide sequence is encoded by a plurality of different polynucleotide sequences. The invention therefore provides variants of the VECSM polynucleotide sequences listed above that can encode the naturally occurring VECSM polypeptides. Such variants may be generated by making codon substitutions in accordance with the genetic code, so that a particular codon substitution replaces a codon with a codon that encodes the same amino acid. It may be advantageous to make select particular codons in order to conform to codon usage patterns in a given host, e.g., to increase the rate of expression in a host in which the polypeptide is to be produced.

The invention further provides VECSM polynucleotide variants whose sequences have at least about 80%, more preferably at least about 90%, and most preferably about 95% nucleotide sequence identity to the naturally occurring VECSM polynucleotides. Certain of the variant polynucleotide sequences differ by less than 50, preferably less than 20, yet more preferably less than 10, and yet more preferably less than 5 nucleotides from the sequence of naturally occurring VECSM polynucleotides. Two polynucleotides “differ by” X amino acids if it is possible to transform one polynucleotide into the other by adding, deleting, or replacing X nucleotides of the first polynucleotide. It is to be understood that the variants need not actually be produced by modifying a VECSM polynucleotide but may be produced by any suitable method.

In general, the VECSM polynucleotide variants further includes any naturally occurring variant of the VECSM polynucleotides described above. As is well known in the art, many genes exist in a variety of allelic forms, each of which may differ in sequence by one or more nucleotides. Such differences may contribute to phenotypic variability or may be silent (i.e., resulting in no change in coding sequence). Naturally occurring allelic variants (e.g., single nucleotide polymorphisms) are useful for a variety of purposes including genomic mapping and the identification of genes that are responsible for particular phenotypes and/or diseases. See generally Glazier, A., et al., “Finding Genes That Underlie Complex Traits”, Science, 298(5602):2345-9, 2002, reviewing methods and standards for identification of genes underlying human Mendelian traits and genetically complex traits in humans and other species. Naturally occurring variants also include alterations (mutations) that change the function or activity of the mRNA or encoded protein in a manner that has deleterious consequences for the organism, and may thus contribute to disease. Detection of such naturally occurring variants may be performed using the teachings herein, thus contributing to the diagnosis and prognosis of disease. Typically allelic variants of a full length VECSM polynucleotide will vary from each other by one, two, or at most a few (e.g., up to 10) nucleotides. Similarly, allelic variants of a full length VECSM polypeptide will vary from each other by one, two, or a few amino acids.

The invention encompasses polynucleotides that are able to hybridize to any of the inventive polynucleotide sequences discussed herein under various conditions of stringency. In general, a hybridizing polynucleotide will have a sequence either partially or fully complementary with the polynucleotide to which it hybridizes. Hybridization conditions of various stringency are well known in the art and are, in general, governed by the concentration of reagents such as salts and formamide in the hybridization buffer as well as by the temperature at which hybridization is performed. For example, a stringent hybridization can be performed by use of a hybridization buffer comprising 30% formamide in 0.9M saline/0.09M sodium citrate (SSC) buffer at a temperature of 45° C. followed by washing twice with that SSC buffer at 45° C. A moderately stringent hybridization condition could include use of a hybridization buffer comprising 20% fornamide in 0.8M saline/0.08M SSC buffer at a temperature of 37° C. followed by washing once with that SSC buffer at 37° C. Other examples are provided in the Definitions and in standard references. The selection of appropriate hybridization conditions will typically be determined by the purpose for which hybridization is to be carried out and is a matter of choice for the practitioner. When the polynucleotide is present on an array, the hybridization conditions may be chosen as described in the Examples.

G. VECSM Polypeptide and Peptide Variants

The invention further provides VECSM polypeptide variants having a sequence that may be obtained by deliberate replacement, deletion, or addition of one or more amino acids from or to a naturally occurring VECSM polypeptide sequence, e.g., the substitution of an amino acid with a different amino acid having similar properties (conservative substitution). Conservative amino acid substitution is well known in the art and represents one approach to obtaining a polypeptide having similar or substantially similar properties to those of a given polypeptide while altering the amino acid sequence. In general, amino acids have been classified and divided into groups according to (1) charge (positive, negative, or uncharged); (2) volume and polarity; (3) Grantham's physico-chemical distance; and combinations of these. See, e.g., Zhang, J., J. Mol. Evol., 50: 56-68, 2000; Grantham R., Science, 85: 862-864, 1974; Dagan, T., et al., Mol. Biol. Evol., 19(7), 1022-1025, 2002; Biochemistry, 4th Ed., Stryer, L., et al., W. Freeman and Co., 1995; and U.S. Pat. No. 6,015,692. For example, amino acids may be divided into the following 6 categories based on volume and polarity: special (C); neutral and small (A, G, P, S, T); polar and relatively small (N, D, Q, E), polar and relatively large (R, H, K), nonpolar and relatively small (I, L, M, V), and nonpolar and relatively large (F, W, Y). According to certain embodiments of the invention a conservative amino acid substitution is one that replaces one amino acid with an amino acid in the same group. The invention provides VECSM polypeptide variants having between 1 and 5 deletions, additions, or conservative substitutions. The invention further provides VECSM polypeptide variants whose sequence has between 6 and 10, between 10 and 15, between 15 and 20, or between 20 and 25 deletions, additions, or conservative substitutions relative to the original VECSM sequence. The variants may have a combination of deletions, additions, or conservative substitutions. It is to be understood that the variants need not actually be produced by modifying a VECSM polypeptide but may be produced by any suitable method.

More broadly, the invention encompasses variant polypeptides whose sequences may be obtained by deliberate replacement, deletion, or addition of one or more amino acids from or to the original polypeptide sequence, wherein the replacement, deletion, or addition results in a polypeptide that retains at least one structural or functional feature of the original polypeptide. Such feature may be recognition by a particular antibody, possession of a biological activity such as an enzyme activity, ability to interact with or bind to another protein, etc. In particular, the invention includes variant polypeptides that differ from the original polypeptide by a single amino acid (e.g., an addition, deletion, or substitution of a single amino acid), variants that differ by between 2 and 5 amino acids, variants that differ by between 6 and 10 amino acids, variants that differ by between 11 and 20 amino acids, and variants that differ by between 21 and 50 amino acids from the original VECSM sequence. Two polypeptides “differ by” X amino acids if it is possible to transform one polypeptide into the other by adding, deleting, or replacing X amino acids of the first polypeptide.

The invention further provides VECSM peptide variants. According to certain embodiments of the invention, the sequence of a VECSM polypeptide or peptide may differ from that of the corresponding VECSM polynucleotide or peptide at not more than 10% of the amino acid positions or, according to certain embodiments of the invention at not more than between 1 and 5 amino acid positions.

The invention also encompasses polypeptide variants possessing significant similarity to VECSM polypeptides, wherein definitions of “significantly similar” make reference to the BLAST algorithm and BLOSUM substitution matrix, which are described in Altschul, SF, et al., “Gapped BLAST and PSI-BLAST: a new generation of protein database search programs”, Nucleic Acids Res. 25:3389-3402, 1997 and Henikoff, S. and Henikoff, J., “Amino acid substitution matrices from protein blocks”, Proc. Natl. Acad. Sci. 89, 10915-10919, 1992. In certain embodiments of the invention a significantly similar polypeptide is encoded by a naturally occurring gene (e.g., an animal gene, more specifically a mammalian gene, e.g., a rodent gene or a human gene).

In certain embodiments of the invention a polypeptide is considered significantly similar to a VECSM polypeptide if, when the amino acid sequence of the polypeptide is compared with the amino acid sequence of the VECSM polypeptide using the BLAST algorithm and the BLOSUM substitution matrix with default parameters, the result is a % identity greater than 60 or a % positive greater than 70 encompassing at least 25% of the length of the VECSM polypeptide or both a % identity and a % positivity satisfying these criteria. In other embodiments of the invention a polypeptide is considered significantly similar to a VECSM polypeptide if, when the amino acid sequence of the polypeptide is compared with the amino acid sequence of the VECSM polypeptide using the BLAST algorithm and the BLOSUM substitution matrix with default parameters, the result is a % identity greater than 60 or a % positive greater than 70 encompassing at least 50% of the length of the VECSM polypeptide or both a % identity and a % positivity satisfying these criteria. In other embodiments of the invention a polypeptide is considered significantly similar to a VECSM polypeptide if, when the amino acid sequence of the polypeptide is compared with the amino acid sequence of the VECSM polypeptide using the BLAST algorithm and the BLOSUM substitution matrix with default parameters, the result is a % identity greater than 60 or a % positive greater than 70 encompassing at least 75% of the length of the VECSM polypeptide, or both a % identity and a % positivity satisfying these criteria. In other embodiments of the invention a polypeptide is considered significantly similar to a VECSM polypeptide if, when the amino acid sequence of the polypeptide is compared with the amino acid sequence of the VECSM polypeptide using the BLAST algorithm and the BLOSUM substitution matrix with default parameters, the result is a % identity greater than 60 or a % positive greater than 70 encompassing at least 90% of the length of the VECSM polypeptide, respectively, or both a % identity and a % positivity satisfying these criteria. In other embodiments of the invention a polypeptide is considered significantly similar to a VECSM polypeptide if, when the amino acid sequence of the polypeptide is compared with the amino acid sequence of the VECSM polypeptide using the BLAST algorithm and the BLOSUM substitution matrix with default parameters, the result is a % identity greater than 60 or a % positive greater than 70 encompassing at least 95% of the length of the VECSM polypeptide, or both a % identity and a % positivity satisfying these criteria.

In other embodiments of the invention a polypeptide is considered significantly similar to a VECSM polypeptide if, when the amino acid sequence of the polypeptide is compared with the amino acid sequence of the VECSM polypeptide using the BLAST algorithm and the BLOSUM substitution matrix with default parameters, the result is a % identity greater than 70 or a % positive greater than 80 encompassing at least 25% of the length of the VECSM polypeptide or both a % identity and a % positivity satisfying these criteria. In other embodiments of the invention a polypeptide is considered significantly similar if the VECSM polypeptide if, when the amino acid sequence of the polypeptide is compared with the amino acid sequence of the polypeptide of the VECSM polypeptide using the BLAST algorithm and the BLOSUM substitution matrix with default parameters, the result is a % identity greater than 70 or a % positive greater than 80 encompassing at least 75% of the length of the VECSM polypeptide or both a % identity and a % positivity satisfying these criteria. In other embodiments of the invention a polypeptide is considered significantly similar to a VECSM polypeptide if, when the amino acid sequence of the polypeptide is compared with the amino acid sequence of the VECSM polypeptide using the BLAST algorithm and the BLOSUM substitution matrix with default parameters, the result is a % identity greater than 70 or a % positive greater than 80 encompassing at least 90% of the length of the VECSM polypeptide or both a % identity and a % positivity satisfying these criteria. In other embodiments of the invention a polypeptide is considered significantly similar if, when the amino acid sequence of the polypeptide is compared with the amino acid sequence of the VECSM polypeptide using the BLAST algorithm and the BLOSUM substitution matrix with default parameters, the result is a % identity greater than 70 or a % positive greater than 80 encompassing at least 95% of the length of the VECSM polypeptide or both a % identity and a % positivity satisfying these criteria.

In other embodiments of the invention a polypeptide is considered significantly similar to a VECSM polypeptide if, when the amino acid sequence of the polypeptide is compared with the amino acid sequence of the VECSM polypeptide using the BLAST algorithm and the BLOSUM substitution matrix with default parameters, the result is a % identity greater than 80 or a % positive greater than 90 encompassing at least 25% of the length of the VECSM polypeptide or both a % identity and a % positivity satisfying these criteria. In other embodiments of the invention a polypeptide is considered significantly similar to a VECSM polypeptide if, when the amino acid sequence of the polypeptide is compared with the amino acid sequence of the VECSM polypeptide using the BLAST algorithm and the BLOSUM substitution matrix with default parameters, the result is a % identity greater than 80 or a % positive greater than 90 encompassing at least 75% of the length of the VECSM polypeptide or both a % identity and a % positivity satisfying these criteria. In other embodiments of the invention a polypeptide is considered significantly similar to a VECSM polypeptide if, when the amino acid sequence of the polypeptide is compared with the amino acid sequence of the polypeptide of the VECSM polypeptide using the BLAST algorithm and the BLOSUM substitution matrix with default parameters, the result is a % identity greater than 80 or a % positive greater than 90 encompassing at least 90% of the length of the VECSM polypeptide or both a % identity and a % positivity satisfying these criteria. In other embodiments of the invention a polypeptide is considered significantly similar to a VECSM polypeptide if, when the amino acid sequence of the polypeptide is compared with the amino acid sequence of the VECSM polypeptide using the BLAST algorithm and the BLOSUM substitution matrix with default parameters, the result is a % identity greater than 80 or a % positive greater than 90 encompassing at least 95% of the length of the VECSM polypeptide or both a % identity and a % positivity satisfying these criteria.

In other embodiments of the invention a polypeptide is considered significantly similar to a VECSM polypeptide if, when the amino acid sequence of the polypeptide is compared with the amino acid sequence of the polypeptide of the VECSM polypeptide using the BLAST algorithm and the BLOSUM substitution matrix with default parameters, the result is a % identity greater than 90 or a % positive greater than 95 encompassing at least 25% of the length of the VECSM polypeptide or both a % identity and a % positivity satisfying these criteria. In other embodiments of the invention a polypeptide is considered significantly similar to a VECSM polypeptide if, when the amino acid sequence of the polypeptide is compared with the amino acid sequence of the polypeptide of the VECSM polypeptide using the BLAST algorithm and the BLOSUM substitution matrix with default parameters, the result is a % identity greater than 90 or a % positive greater than 95 encompassing at least 75% of the length of the VECSM polypeptide or both a % identity and a % positivity satisfying these criteria. In other embodiments of the invention a polypeptide is considered significantly similar to a VECSM polypeptide if, when the amino acid sequence of the polypeptide is compared with the amino acid sequence of the polypeptide of the VECSM polypeptide using the BLAST algorithm and the BLOSUM substitution matrix with default parameters, the result is a % identity greater than 90 or a % positive greater than 95 encompassing at least 90% of the length of S the VECSM polypeptide or both a % identity and a % positivity satisfying these criteria. In other embodiments of the invention a polypeptide is considered significantly similar to a VECSM polypeptide if, when the amino acid sequence of the polypeptide is compared with the amino acid sequence of the polypeptide of the VECSM polypeptide using the BLAST algorithm and the BLOSUM substitution matrix with default parameters, the result is a % identity greater than 90 or a % positive greater than 95 encompassing at least 95% of the length of the VECSM polypeptide, respectively, or both a % identity and a % positivity satisfying these criteria. By “encompassing at least X% of the length of a VECSM polypeptide is meant that the length of the portion of the VECSM polypeptide that is being compared with a potentially similar protein is at least X% of the length of the VECSM polypeptide. The invention further provides polynucleotides that encode the VECSM polypeptide variants and polynucleotides complementary to such polynucleotides.

H. VECSM Polypeptide Fragments

The invention also includes fragments of VECSM polypeptides (referred to as “VECSM polypeptide fragments”), e.g., fragments of the VECSM polypeptides described above, e.g. Group III VECSM polypeptides. In certain embodiments of the invention the fragments are between 8 and 50 amino acids in length, inclusive. In certain embodiments of the invention the fragments are between 51 and 100 amino acids in length, inclusive. In certain embodiments of the invention the fragments are between 101 and 200 amino acids in length, inclusive. In certain embodiments of the invention the fragments are between 201 and 300 amino acids in length, inclusive. In certain embodiments of the invention the fragments are between 301 and 500 amino acids in length, inclusive. In certain embodiments of the invention the fragments are between 501 and 1000 amino acids in length, inclusive. Such fragments are useful, for example, as antigens for the generation of antibodies. In addition, fragments containing functionally active domains may be more easily produced than full length polypeptides, which may provide advantages when using such molecules for treatment, etc. In certain embodiments of the invention the length of the a VECSM polypeptide fragment is at least 50%, at least 75%, at least 90%, at least 95%, or at least 99% of the full length of the VECSM polypeptide or VECSM polypeptide variant. The invention further provides polynucleotides that encode the VECSM polypeptide fragments and polynucleotides complementary to such polynucleotides.

I. VECSM Polynucleotide and Polypeptide Analogs

According to certain embodiments of the invention the VECSM polynucleotides described above consist entirely of nucleotides that occur naturally within DNA or RNA in the species from which the gene originates (e.g., in mammals, preferably humans). However, the invention also provides VECSM polynucleotide analogs, i.e., polynucleotides having a VECSM polynucleotide sequence, which polynucleotides comprise one or more altered or modified nucleotides, nucleotide analogs, or nucleotides that are not normally found within the species from which the gene originates. In general, modifications may be made in the base, sugar, and/or backbone portion(s) of the polynucleotide. Numerous modifications are known in the art, and a number of these are known to confer particular properties (e.g., nuclease resistance) to polynucleotides that contain them. See discussion below in the section describing antisense molecules.

The invention also provides VECSM polypeptide analogs, i.e., polypeptides having a VECSM polypeptide sequence which polypeptides incorporate one or more unnatural amino acids (e.g., amino acids that are not naturally found in the species in which the VECSM polypeptide originates, or amino acids that are not naturally found in any organism). Such amino acids include, but are not limited to, cyclic amino acids, diamino acids, β-amino acids, homo amino acids, alanine derivatives, phenylalanine boronic acids, proline and pyroglutamine derivatives, etc. Alterations and modifications may include the replacement of an L- amino acid with a D-amino acid, or various modifications including, but not limited to, phosphorylation, carboxylation, alkylation, etc.

Polypeptides incorporating unnatural amino acids may be produced either entirely artificially or through biological processes, e.g., in living organisms. Use of unnatural amino acids may have a number of advantages. For example, unnatural amino acids may be utilized as building blocks, conformational constraints, molecular scaffolds, or pharmacologically active products. They represent a broad array of diverse structural elements that may be utilized, e.g., for the development of new leads in peptidic and non-peptidic compounds. They may confer desirable features such as enhanced biological activity, proteolytic resistance, etc. See, e.g., Bunin, B. A. et al., Annu. Rep. Med. Chem. 1999, 34, 267; Floyd, C. D. et al., Prog. Med. Chem. 1999, 36, 91; Borman, S. Chem. Eng. News 1999, 77, 33; Brown, R. K Modern Drug Discovery 1999, 2, 63; and Borman, S. Chem. Eng. News 2000, 78, 53, describing various applications of unnatural amino acids.

II. Identification of Full Length cDNAs and Encoded Polypeptides

Full length cDNAs may be identified according to a variety of approaches well known in the art. Additional nucleotide sequence can be obtained by identifying and sequencing clones, e.g., ESTs in the same Unigene cluster and/or I.M.A.G.E. clones having sequences that overlap with those of the clones used on the microarray, which are available from Invitrogen, Carlsbad, Calif. (See Lennon, G., Auffray, C., Polymeropoulos, M., Soares, M. B., “The I.M.A.G.E. Consortium: an integrated molecular analysis of genomes and their expression”, Genomics 33, 151-152, 1996). These nucleotide sequences can be used to construct a consensus sequence. Any of these nucleotide sequences may be extended by using any of a variety of techniques well known in the art. For example, reverse transcription PCR (RT-PCR) using primers based on known sequence may be used to identify longer cDNA clones. A variety of other methods such as restriction site PCR, inverse PCR, and capture PCR, are discussed in U.S. Pat. No. 6,008,337. Extended sequence obtained thereby may include upstream sequences such as promotors and other regulatory elements. Genomic sequence such as introns can also be obtained.

Discovery of additional sequence may also be performed using computer-based searches of sequenced human DNA. For example, the availability of databases containing the complete human genome sequence allows the precise chromosomal location of the clone to be identified, together with the sequence of DNA upstream and downstream. Analysis of reading frames and putative start and stop sites for transcription allows assembly of a candidate full length cDNA sequence. The identity of the full length cDNA may be confirmed, and a full length clone obtained, for example, by performing reverse transcription PCR (RT-PCR) on cells or tissue samples in which the gene is expected to be expressed (e.g., endothelial cells in the case of the genes described herein). Other approaches may also be used, e.g., probing a cDNA library prepared from cells or tissue samples in which the gene is expected to be expressed. Alternatively, a full length cDNA may be chemically synthesized, although it is generally preferable to confirm results obtained from database searching with actual cloning.

Of course it is not necessary to perform database searching prior to RT-PCR (or other approaches) in order to identify a full length cDNA. Either RT-PCR or other suitable approaches may be used without the additional information available from databases. It is noted that these approaches may also be used to identify and isolate cDNAs that, while longer than a clone itself, may be shorter than the full length cDNA corresponding to the clone.

As is well known in the art it is straightforward, based on the genetic code and the correspondence between amino acids and triplet codons which it defines, to derive a corresponding polypeptide sequence from each polynucleotide sequence. In most cases the amino acid sequence of the polypeptide encoded by a cDNA may be readily determined by analytic approaches that include: (i) identifying start codons; (ii) identifying the longest open reading frame (ORF) in the cDNA; (iii) identifying the putative translation initiation site. In cases where sequence analysis is not sufficient to determine which of multiple possible polypeptides is produced, anti-peptide antibodies may be raised, each of which is capable of binding to only a single polypeptide among the possibilities. These antibodies may be used to determine which of the candidate polypeptides is actually present in endothelial cells. The invention therefore provides a polypeptide encoded by a cDNA complementary to a naturally occurring mRNA, wherein the cDNA corresponds to a polynucleotide. having a sequence identified by accession number in Table 2 or Table 3, wherein the length of the cDNA is greater than the length of the polynucleotide.

J. Using VECSM Regulatory Elements for Tissue-Specific Expression

VECSM promoters and other regulatory elements may be isolated and used to direct expression of operatively linked polynucleotides in endothelial cells. For example, a VECSM promoter may be inserted into a vector upstream from and operatively linked to a polynucleotide sequence of interest so that the promoter directs expression of the polynucleotide in an appropriate host cell, e.g., an endothelial cell. The invention therefore provides a method of expressing a polynucleotide in an endothelial cell comprising steps of (i) operatively linking the polynucleotide to a VECSM promoter element, thereby generating an endothelial cell specific expression cassette; and (ii) introducing the expression cassette into a host cell. The VECSM regulatory elements may be used to direct endothelial-cell specific expression of a polynucleotide of interest in a transgenic non-humna animal, e.g., a mouse.

III. Antisense Nucleic Acids, Ribozymes, and Short Interfering RNAs

A. VECSM Antisense Nucleic Acids and Methods of Use

Antisense nucleic acids are generally single-stranded nucleic acids (DNA, RNA, modified DNA, or modified RNA) complementary to a portion of a target nucleic acid (e.g., an mRNA transcript) and therefore able to bind to the target to form a duplex. Typically they are oligonucleotides that range from 15 to 35 nucleotides in length but may range from 10 up to approximately 50 nucleotides in length. Binding typically reduces or inhibits the function of the target nucleic acid. For example, antisense oligonucleotides may block transcription when bound to genomic DNA, inhibit translation when bound to mRNA, and/or lead to degradation of the nucleic acid. Reduction in expression of a VECSM polypeptide may be achieved by the administration of antisense nucleic acids or peptide nucleic acids comprising sequences complementary to those of the mRNA that encodes the polypeptide. Antisense technology and its applications are well known in the art and are described in Phillips, M. I. (ed.) Antisense Technology, Methods Enzymol., Volumes 313 and 314, Academic Press, San Diego, 2000, and references mentioned therein. See also Crooke, S. (ed.) “Antisense Drug Technology: Principles, Strategies, and Applications” (1^{st} ed), Marcel Dekker; ISBN: 0824705661; 1st edition (2001) and references therein.

The invention provides a variety of different VECSM antisense oligonucleotides. As used herein, the term “VECSM antisense oligonucleotide” includes any VECSM oligonucleotide that comprises between 8 and 60 contiguous nucleotides complementary to a VECSM mRNA. According to certain embodiments of the invention a VECSM antisense oligonucleotide comprises between 10 and 60 contiguous nucleotides complementary to a VECSM mRNA. According to certain embodiments of the invention a VECSM antisense oligonucleotide comprises between 12 and 60 contiguous nucleotides complementary to a VECSM mRNA. According to certain embodiments of the invention a VECSM antisense olignucleotide need not be perfectly complementary to the corresponding mRNA but may have up to 1 or 2 mismatches per 10 nucleotides when hybridized to the corresponding mRNA. In certain embodiments of the invention the total length of the antisense oligonucleotide is less than 30 nucleotides. In certain embodiments of the invention the total length of the antisense oligonucleotide is between 15 and 30 nucleotides.

The invention further encompasses a method of inhibiting expression of a VECSM polypeptide in a cell or a subject comprising delivering a VECSM antisense oligonucleotide to the cell or subject. In addition, the invention provides a method of treating a condition associated with inappropriate or excessive vascular endothelial growth comprising steps of (i) providing a subject in need of treatment for a condition associated with inappropriate or excessive vascular endothelial growth; and (ii) administering a pharmaceutical composition comprising an effective amount of a VECSM antisense oligonucleotide to the subject, thereby alleviating the condition. In addition, the invention provides a method of treating a condition associated with an inadequate or reduced blood supply or inadequate or reduced angiogenesis or vascular endothelial growth comprising steps of (i) providing a subject in need of treatment for a condition associated with an inadequate or reduced blood supply or inadequate or reduced angiogenesis or vascular endothelial growth; and (ii) administering a pharmaceutical composition comprising an effective amount of a VECSM antisense oligonucleotide to the subject, thereby alleviating the condition.

B. VECSM Ribozymes and Methods of Use

Ribozymes (catalytic RNA molecules that are capable of cleaving other RNA molecules) represent another approach to reducing gene expression. Such ribozymes can be designed to cleave specific mRNAs corresponding to a gene of interest. Their use is described in U.S. Pat. No. 5,972,621, and references therein. The invention provides a ribozyme designed to cleave VECSM mRNA. The invention further encompasses a method of inhibiting expression of a VECSM polypeptide in a cell or subject comprising delivering a ribozyme designed to cleave VECSM mRNA to the cell or subject. In addition, the invention provides a method of treating a condition associated with inappropriate or excessive vascular endothelial growth comprising steps of (i) providing a subject in need of treatment for a condition associated with inappropriate or excessive vascular endothelial growth; and (ii) administering a pharmaceutical composition comprising an effective amount of a ribozyme designed to cleave VECSM mRNA to the subject, thereby alleviating the condition. In addition, the invention provides a method of treating a condition associated with an inadequate or reduced blood supply or inadequate or reduced angiogenesis or vascular endothelial growth comprising steps of (i) providing a subject in need of treatment for a condition associated with an inadequate or reduced blood supply or inadequate or reduced angiogenesis or vascular endothelial growth; and (ii) administering a pharmaceutical composition comprising an effective amount of a ribozyme designed to cleave VECSM mRNA to the subject, thereby alleviating the condition.

C. RNAi-Inducing Agents Targeted to VECSmRNA and Methods of Use

RNA interference (RNAi) is a mechanism of post-transcriptional gene silencing mediated by double-stranded RNA (dsRNA), which is distinct from the antisense and ribozyme-based approaches described above. dsRNA molecules are believed to direct sequence-specific degradation of mRNA in cells of various types after first undergoing processing by an RNase III-like enzyme called DICER (Bernstein et al., Nature 409:363, 2001) into smaller dsRNA molecules comprised of two 21 nt strands, each of which has a 5′ phosphate group and a 3′ hydroxyl, and includes a 19 nt region precisely complementary with the other strand, so that there is a 19 nt duplex region flanked by 2 nt-3′ overhangs. RNAi is thus mediated by short interfering RNAs (siRNA), which typically comprise a double-stranded region approximately 19 nucleotides in length with 1-2 nucleotide 3′ overhangs on each strand, resulting in a total length of between approximately 21 and 23 nucleotides. In mammalian cells, dsRNA longer than approximately 30 nucleotides typically induces nonspecific mRNA degradation via the interferon response. However, the presence of siRNA in mammalian cells, rather than inducing the interferon response, results in sequence-specific gene silencing.

siRNA has been shown to downregulate gene expression when transferred into mammalian cells by such methods as transfection, electroporation, or microinjection, or when expressed in cells via any of a variety of plasmid-based approaches. RNA interference using siRNA is reviewed in, e.g., Tuschl, T., Nat. Biotechnol., 20: 446-448, May 2002. See also Yu, J., et al., Proc. Natl. Acad. Sci., 99(9), 6047-6052 (2002); Sui, G., et al., Proc. Natl. Acad. Sci., 99(8), 5515-5520 (2002); Paddison, P., et al., Genes and Dev., 16, 948-958 (2002); Brummelkamp, T., et al., Science, 296, 550-553 (2002); Miyagashi, M. and Taira, K., Nat. Biotech., 20, 497-500 (2002); Paul, C., et al., Nat. Biotech., 20, 505-508 (2002). As described in these and other references, RNAi may achieved using an siRNA consisting of two individual nucleic acid strands or using a single RNA strand with a self-complementary region capable of forming a hairpin (stem-loop) structure (short hairpin RNA). A number of variations in structure, length, number of mismatches, size of loop, identity of nucleotides in overhangs, etc., are consistent with effective siRNA-triggered gene silencing. While not wishing to be bound by any theory, it is thought that intracellular processing (e.g., by DICER) of a variety of different precursors results in production of siRNA capable of effectively mediating gene silencing. Generally it is preferred to target exons rather than introns, and it may also be preferable to select sequences complementary to regions within the 3′ portion of the target transcript. Generally it is preferred to select sequences that contain approximately equimolar ratio of the different nucleotides and to avoid stretches in which a single residue is repeated multiple times.

An siRNA may thus comprise an RNA structure having a double-stranded region approximately 19 nucleotides in length with 1-2 nucleotide 3′ overhangs on each strand, resulting in a total length of between approximately 21 and 23 nucleotides. siRNA may be generated intracellularly from various RNA structures (short hairpin RNAs) that contain two complementary elements that hybridize to one another to form a stem, a loop, and optionally an overhang, preferably a 3′ overhang. Such shRNAs are also provided by the invention and can be produced exogenously. Preferably, the stem is approximately 19 bp long, the loop is about 1-20, more preferably about 4-10, and most preferably about 6-8 nt long and/or the overhang is about 1-20, and more preferably about 2-15 nt long. In certain embodiments of the invention the stem is minimally 19 nucleotides in length and may be up to approximately 29 nucleotides in length. Loops of 4 nucleotides or greater are less likely subject to steric constraints than are shorter loops and therefore may be preferred. The overhang may include a 5′ phosphate and a 3′ hydroxyl. The overhang may but need not comprise a plurality of U residues, e.g., between 1 and 5 U residues.

Accordingly, the invention provides siRNA and shRNA compositions targeted to mRNA encoding any of the VECSM polypeptides. The siRNAs and shRNAs of the invention may be prepared by any method known in the art for the synthesis of nucleic acid molecules. These include techniques for chemical synthesis such as solid phase phosphoramidite chemical synthesis. Inventive siRNAs or shRNAs may be comprised entirely of natural RNA nucleotides, or may instead include one or more nucleotide analogs and/or modifications as mentioned above for antisense molecules. The RNA structure may be stabilized, for example by including nucleotide analogs at one or more free strand ends in order to reduce digestion, e.g., by exonucleases. This may also be accomplished by the use of deoxy residues at the ends, e.g., by employing dTdT overhangs at one or more 3′ ends. Alternatively, siRNA or shRNA molecules may be generated by in vitro transcription of DNA sequences encoding the relevant molecule. Such DNA sequences may be incorporated into a wide variety of vectors with suitable RNA polymerase promoters such as T7, T3, or SP6.

siRNA or shRNA may be generated by intracellular transcription of small RNA molecules, which may be followed by intracellular processing events. For example, intracellular transcription is achieved by cloning siRNA or shRNA templates into RNA polymerase III transcription units, e.g., under control of a U6 or HI promoter. In one approach, sense and antisense strands are transcribed from individual promoters, which may be on the same construct. The promoters may be in opposite orientation so that they drive transcription from a single template, or they may direct synthesis from different templates. Alternately, shRNAs may be expressed as stem-loop structures. The siRNAs, shRNAs, or vectors providing templates for synthesis of one or more siRNAs or shRNAs (RNAi-inducing vector) may be introduced into cells by any of a variety of methods. For instance, siRNAs or RNAi-inducing vector the can be introduced into cells via conventional transformation or transfection techniques. As used herein, the terms “transformation” and “transfection” are intended to refer to a variety of art-recognized techniques for introducing foreign nucleic acid (e.g., DNA or RNA) into a host cell, including calcium phosphate or calcium chloride co-precipitation, DEAE-dextran-mediated transfection, lipofection, injection, or electroporation. It is noted that it may be desirable to utilize siRNA rather than shRNA when it is desirable to cause RNAi by exogenous administration of RNA, while it may be desirable to utilize vectors that provide templates for transcription of shRNA rather than siRNA.

Vectors that direct in vivo synthesis of siRNA or shRNA constitutively or inducibly can be introduced into cell lines, cells, or tissues. In certain preferred embodiments of the invention, inventive vectors are gene therapy vectors (e.g., adenoviral vectors, adeno-associated viral vectors, retroviral or lentiviral vectors, or various nonviral gene therapy vectors) appropriate for the delivery of a construct directing transcription of an siRNA to mammalian cells, most preferably human cells.

The invention further encompasses a method of inhibiting expression of a VECSM gene in a cell or subject comprising delivering an siRNA, shRNA, or RNAi-inducing vector targeted to VECSM mRNA to the cell or subject. In addition, the invention provides a method of treating a condition associated with inappropriate or excessive vascular endothelial growth comprising steps of (i) providing a subject in need of treatment for a condition associated with inappropriate or excessive vascular endothelial growth; and (ii) administering a pharmaceutical composition comprising an siRNA, shRNA, or RNAi-inducing vector targeted to VECSM mRNA to the subject, thereby alleviating the condition. In addition, the invention provides a method of treating a condition associated with an inadequate or reduced blood supply or inadequate or reduced angiogenesis or vascular endothelial growth comprising steps of (i) providing a subject in need of treatment for a condition associated with an inadequate or reduced blood supply or inadequate or reduced angiogenesis or vascular endothelial growth; and (ii) administering a pharmaceutical composition comprising an siRNA, shRNA, or RNAi-inducing vector targeted to VECSM mRNA to the subject, thereby alleviating the condition. The invention provides similar methods for other RNAi-inducing compositions.

Preferred RNAi-inducing compositions (e.g., siRNA, shRNA, or vectors providing templates for synthesis of siRNA or shRNA) reduce the level of the target transcript and its encoded protein by at least 2-fold, preferably at least 4-fold, more preferably at least 10-fold or more. The ability of a candidate siRNA, shRNA, or RNAi-inducing vector to reduce expression of the target transcript and/or its encoded protein may readily be tested using methods well known in the art including, but not limited to, Northern blots, RT-PCR, microarray analysis in the case of the transcript, and various immunological methods such as Western blot, ELISA, immunofluorescence, etc., in the case of the encoded protein. In addition, the potential of any siRNA composition for treatment of a particular condition or disease associated with inappropriate or excessive vascular endothelial growth or an inadequate or reduced blood supply or inadequate or reduced angiogenesis or vascular endothelial growth. Efficacy may also be tested in appropriate animal models or in human subjects, as is the case for all methods of treatment described herein.

D. Delivery Methods and Modifications

Antisense nucleic acids, ribozymes, or siRNAs, shRNAs, or RNAi-inducing vectors can be delivered to cells by standard techniques such as microinjection, electroporation, or transfection. Antisense nucleic acids, ribozymes, siRNAs, shRNAs, or RNAi-inducing vectors can be formulated as pharmaceutical compositions and delivered to a subject using a variety of approaches, as described further below. According to certain embodiments of the invention the delivery of antisense, ribozyme, siRNA, shRNA, or RNAi-inducing vector molecules to a subject is accomplished via a gene therapy approach in which vectors (e.g., viral vectors such as retroviral, lentiviral, or adenoviral vectors, etc.) or cells directing expression of the molecules administered to the subject. Delivery methods are discussed further below.

It may advantageous to employ various nucleotide modifications and analogs to confer desirable properties on the antisense nucleic acid, ribozyme, siRNA, or shRNA. Numerous nucleotide analogs and nucleotide modifications are known in the art, and their effect on properties such as hybridization and nuclease resistance has been explored. For example, various modifications to the base, sugar and internucleoside linkage have been introduced into oligonucleotides at selected positions, and the resultant effect relative to the unmodified oligonucleotide compared. A number of modifications have been shown to alter one or more aspects of the oligonucleotide such as its ability to hybridize to a complementary nucleic acid, its stability, etc . For example, useful 2′-modifications include halo, alkoxy and allyloxy groups. U.S. Pat. Nos. 6,403,779; 6,399,754; 6,225,460; 6,127,533; 6,031,086; 6,005,087; 5,977,089, and references therein disclose a wide variety of nucleotide analogs and modifications that may be of use in the practice of the present invention. See also Crooke, S. (ed.), referenced above, and references therein. As will be appreciated by one of ordinary skill in the art, analogs and modifications may be tested using, e.g., the assays described herein or other appropriate assays, in order to select those that effectively reduce expression of the target nucleic acid.

In certain embodiments of the invention the analog or modification results in a nucleic acid with increased absorbability (e.g., increased absorbability across a mucus layer, increased oral absorption, etc.), increased stability in the blood stream or within cells, increased ability to cross cell membranes, etc. The invention encompasses antisense nucleic acids, ribozymes, and siRNAs incorporating any of these or other known useful nucleic acid modifications and/or analogs.

IV. Vectors, Host Cells, Transgenic and Knockout Animals

The invention provides plasmids and other vectors comprising the VECSM polynucleotides, VECSM polypeptide variants, and VECSM polypeptide fragments, and cells comprising such vectors. Vectors comprising VECSM polynucleotides, variants, and fragments may be introduced into any of a wide variety of cell types using, e.g., transformation or conjugation (for bacteria or yeast), electroporation, transfection (for insect or mammalian cells), or other forms of gene transfer. Such gene transfer may be performed, e.g., in order to study the effect of expressing the polypeptide in the cell, for therapeutic purposes (gene therapy), in order to produce and subsequently isolate the polypeptide, etc. A wide variety of vector/cell expression systems are known in the art. In general, vectors contain necessary control and regulatory sequences or elements (e.g., enhances, promotors, polyadenylation sequence, etc.) operatively linked to an inserted polynucleotide so as to direct expression of the polynucleotide and its encoded polypeptide in the appropriate cell. Depending upon the cell to be employed, appropriate vectors may include phages, viruses, or plasmids. One of ordinary skill in the art will readily be able to select appropriate regulatory elements depending upon the particular host cell (or animal or plant) into which the vector is to be introduced. Suitable host cells widely used to express polypeptides include, but are not limited to, bacteria, yeast, insect cells, and mammalian cells. The invention encompasses any available vector/cell system and specifically includes vectors that direct expression of a VECSM polynucleotide, vectors that direct expression of a VECSM polypeptide, variant, or fragment (e.g., expression vectors), in addition to cells and cell lines comprising, e.g., transformed with, such vectors.

The invention further provides cells and non-human animals (e.g., rodents and other mammals, fish, etc.) and plants that are transgenic for any of the VECSM polynucleotides. In addition to their potential use as sources for VECSM polypeptides, transgenic animals may be used to study the function of the inventive polypeptides. Methods for the production of transgenic animals and plants, as well as methods for purifying and harvesting inventive polypeptides from such animals and plants are well known in the art and are within the scope of the invention. According to certain embodiments of the invention the transgenic animals express the VECSM polynucleotide of interest in a conditional, tissue-specific and/or inducible manner. The invention also encompasses cells and transgenic animals that have been engineered to lack expression of the inventive polynucleotides. Methods for “knocking out” a gene using the technique of homologous recombination and methods of creating cells and organisms lacking expression of the knocked out gene are well known in the art and are described, for example, in U.S. Pat. No. 5,464,764, U.S. Pat. No. 5,487,992, U.S. Pat. No. 5,627,059, and U.S. Pat. No. 5,631,153. In general, mice that over-express or under-express any of the VECSM genes may be generated according to a variety of conventional, recently developed, or emerging transgenic or knockout techniques. Such techniques may include use of cell or tissue specific regulatory elements, inducible systems, etc. See, e.g., Kwan, K., “Conditional alleles in mice: practical considerations for tissue-specific knockouts.” Genesis, 32(2): 49-62, 2002; Lewandowski, M., “Conditional control of gene expression in the mouse”, Nat. Rev. Genet., 2(10): 743-55, 2001; Bockamp, E., et al., Physiol Genomics 11(3):115-32 (2002). While not wishing to be bound by any theory, it is possible that knockout mice that lack expression of a VECSM polynucleotide will display alterations, e.g., defects, in angiogenesis and/or in endothelial cell development. Such mice are therefore useful to study these processes and to screen for compounds that alter angiogenesis and/or endothelial cell development for therapeutic purposes.

V. Delivery of Nucleic Acids to a Subject

The various nucleic acids described above (e.g., nucleic acids encoding VECSM polypeptides, fragments, and variants; antisense oligonucleotides complementary to VECSM mRNA, ribozymes designed to cleave VECSM mRNA, siRNA, shRNA targeted to VECSM mRNA may be delivered to a subject using any of a variety of approaches, including those applicable to non-nucleic acid agents such as IV, intranasal, oral, etc. However, according to certain embodiments of the invention the nucleic acids are delivered via a gene therapy approach, in which a construct capable of directing expression of one or more of the inventive nucleic acids is delivered to cells or to the subject (ultimately to enter cells, where transcription may occur). Thus according to certain embodiments of the invention the vectors described above include gene therapy vectors appropriate for the delivery of a construct that directs expression of a VECSM polypeptide, variant, fragment, etc., or a construct directing transcription of an antisense oligonucleotide complementary to a VECSM mRNA, or a ribozyme designed to cleave VECSM mRNA, or an siRNA targeted to a VECSM mRNA to mammalian cells, preferably domesticated mammal cells, and most preferably human cells. A variety of gene therapy vectors are known in the art. Suitable gene therapy vectors include viral vectors such as adenoviral or adeno-associated viral vectors, retroviral vectors and lentiviral vectors. In certain instances lentiviruses may be preferred due, e.g., to their ability to infect nondividing cells. See, e.g., Mautino and Morgan, AIDS Patient Care STDS 2002 Jan;16(1):11-26. See also Lois, C., et al., Science, 295: 868-872, Feb. 1, 2002, describing the FUGW lentiviral vector; Somia, N., et al. J. Virol 74(9): 4420-4424, 2000; Miyoshi, H., et al., Science 283: 682-686, 1999; and U.S. Pat. No. 6,013,516.

A number of nonviral vectors and gene delivery systems exist, any of which may be used in the practice of the invention. For example, extrachromosomal DNA (e.g., plasmids) may be used as a gene therapy vector. See, e.g., Stoll, S. and Calor, M, “Extrachromosomal plasmid vectors for gene therapy”, Curr Opin Mol Ther, 4(4):299-305, 2002. According to one approach, the inclusion of appropriate genetic elements from various papovaviruses allows plasmids to be maintained as episomes within mammalian cells. Such plasmids are faithfully distributed to daughter cells. In particular, viral elements of various polyomaviruses and papillomaviruses such as BK virus (BKV), bovine papilloma virus 1 (BPV-1) and Epstein-Barr virus (EBV), among others, are useful in this regard. The invention therefore provides plasmids that direct expression of a VECSM polypeptide, variant, fragment, etc., or a construct directing transcription of an antisense oligonucleotide complementary to a VECSM mRNA, or a ribozyme designed to cleave VECSM mRNA, or an siRNA or shRNA targeted to a VECSM mRNA to mammalian cells, preferably domesticated mammal cells, and most preferably human cells. According to certain embodiments of the invention the plasmids comprise a viral element sufficient for stable maintenance of the transfer plasmid as an episome within mammalian cells. Appropriate genetic elements and their use are described, for example, in Van Craenenbroeck, et al., Eur. J Biochem. 267, 5665-5678 (2000) and references therein, all of which are incorporated herein by reference. Plasmids can be delivered as “naked DNA” or in conjunction with a variety of delivery vehicles.

Protein/DNA polyplexes represent an approach useful for delivery of nucleic acids to cells and subjects. These vectors may be used to deliver constructs directing transcription of the inventive nucleic acids (constructs that direct transcription of VECSM polypeptides, fragments, or variants, antisense molecules, ribozymes, siRNAs or shRNAs) or may be used to deliver the nucleic acids themselves. Thus their use is not limited to gene therapy. See, e.g., Cristiano, R., Surg. Oncol. Clin. N. Am., 11(3), 697-715, 2002. Cationic polymers and liposomes may also be used for these purposes. See, e.g., Merdan, T., et al., “Prospects for cationic polymers in gene and oligonucleotide therapy against cancer”, Adv Drug Deliv Res, 54(5), 715-58, 2002; Liu, F. and Huang, L., “Development of non-viral vectors for systemic gene delivery”, J. Control. Release, 78(1-3):259-66, 2002; Maurer, N., et al., “Developments in liposomal drug delivery systems”, Expert Opin Biol Ther, 1(2), 201-26, 2001; and Li, S. and Ma, Z., “Nonviral gene therapy”, Curr Gene Ther, 1(2), 201-26, 2001. See Rasmussen, H., Curr Opin Mol. Ther, 4(5), 476-81, 2002 for a review of angiogenic gene therapy strategies for the treatment of cardiovascular disease. Numerous reagents and methods for gene therapy are described in Philips, I., (ed.), Methods in Enzymology, Vol. 346: Gene Therapy Methods, Academic Press, 2002.

Any of the nucleic acid delivery vehicles (or nucleic acids themselves) can be targeted for delivery to specific cells, tissues, etc. In particular, they can be targeted to endothelial cells using antibodies or ligands that specifically bind to a VECSM polypeptide as discussed further below. Nucleic acids can be directly conjugated to such antibodies or ligands, which then deliver the nucleic acids to endothelial cells.

Gene therapy protocols may involve administering an effective amount of a gene therapy vector comprising a nucleic acid capable of directing expression of a VECSM polynucleotide, variant, or fragment, VECSM antisense nucleic acid, or a ribozyme, siRNA, or shRNA targeted to a VECSM mRNA to a subject. Another approach that may be used alternatively or in combination with the foregoing is to isolate a population of cells, e.g., stem cells or immune system cells from a subject, optionally expand the cells in tissue culture, and administer a gene therapy vector to the cells in vitro. The cells may then be returned to the subject. Optionally, cells expressing the desired polynucleotide, siRNA, shRNA, etc., can be selected in vitro prior to introducing them into the subject. In some embodiments of the invention a population of cells, which may be cells from a cell line or from an individual who is not the subject, can be used. Methods of isolating stem cells, immune system cells, etc., from a subject and returning them to the subject are well known in the art. Such methods are used, e.g., for bone marrow transplant, peripheral blood stem cell transplant, etc., in patients undergoing chemotherapy.

In yet another approach, oral gene therapy may be used. For example, U.S. Pat. No. 6,248,720 describes methods and compositions whereby genes under the control of promoters are protectively contained in microparticles and delivered to cells in operative form, thereby achieving noninvasive gene delivery. Following oral administration of the microparticles, the genes are taken up into the epithelial cells, including absorptive intestinal epithelial cells, taken up into gut associated lymphoid tissue, and even transported to cells remote from the mucosal epithelium. As described therein, the microparticles can deliver the genes to sites remote from the mucosal epithelium, i.e. can cross the epithelial barrier and enter into general circulation, thereby transfecting cells at other locations.

The invention also encompasses the use of VECSM polynucleotides, polynucleotide fragments or variants as DNA vaccines. Such vaccines comprise polynucleotide sequences, typically inserted into vectors, that direct the expression of an antigenic polypeptide within the body of the individual being immunized. Details regarding the development of vaccines, including DNA vaccines, for various forms of cancer may be found, for example, in Brinckerhoff L. H., Thompson L. W., Slingluff C. L., Jr., Melanoma Vaccines, Curr Opin Oncol, 12(2):163-73, 2000 and in Stevenson, F. K., DNA vaccines against cancer: from genes to therapy, Ann. Oncol., 10(12): 1413-8, 1999 and references therein. DNA vaccines comprising VECSM polypeptides, fragments, or variants may be usefil to inhibit angiogenesis, e.g., in the treatment of cancer.

VI. Producing Polynucleotides and Polypeptides

The invention contemplates the production of any of the VECSM polynucleotides and other polynucleotides described herein by chemical synthesis, by PCR, by in vitro transcription (e.g., using SP6 or T7 in vitro transcription systems) or by use of expression vectors comprising the polynucleotides operably linked to a promoter as described above. The invention encompasses the production of either DNA or RNA having the sequence of an inventive polynucleotide. One of ordinary skill in the art will be able to select appropriate vectors and synthesis conditions depending upon whether it is desired to produce DNA or RNA. The polynucleotides can be labeled or conjugated with detectable moieties including radionuclides, enzymes, chromogenic substrates, fluorescent substances, etc., using any of a variety of techniques.

The invention contemplates production of any of the VECSM polypeptides or other polypeptides described herein using any of a variety of techniques including both in vivo or in vitro synthesis. For example, VECSM polypeptides can be chemically synthesized, produced using in vitro translation systems (e.g., rabbit reticulocyte lysate, wheat germ, etc.) Polynucleotides encoding a VECSM polypeptide can be inserted into an expression vector, which can then be introduced into an appropriate host cell (e.g., a bacterial, yeast, insect, or mammalian cell). As mentioned above, the invention provides an expression vector comprising a VECSM polynucleotide, and a host cell comprising such a vector. In certain embodiments of the invention the inventive polypeptide is secreted from a cell transformed with an expression vector, thereby allowing purification from the medium rather than by harvesting the cells. The invention also encompasses the production of inventive polypeptides in cells that have been engineered to express such polypeptides according to the methods described in U.S. Pat. No. 6,063,630, which discloses methods of “turning on” an endogenous gene in cells that normally express the gene at low or undetectable levels. Methods for harvesting, isolating, purifying, etc., polypeptides from cells expressing such polypeptides are well known in the art.

The invention therefore provides a method of producing a VECSM polypeptide comprising steps of (i) introducing an expression vector comprising a VECSM nucleotide sequence operably linked to a promoter into a suitable host cell cultured in a culture medium; (ii) maintaining the host cell under conditions suitable for expression of the polypeptide; (iii) harvesting the cell or culture medium; and (iv) isolating the VECSM polypeptide from the cell or culture medium. The inventive polypeptides can also be produced in animals or plants that are transgenic for a corresponding polynucleotide sequence. Isolated VECSM polypeptides may be purified and employed for any of a variety of purposes including as antigens and as therapeutic agents.

VII. VECSM Fusion Proteins

As will be appreciated by one of ordinary skill in the art, in certain circumstances it may be advantageous to modify an inventive polynucleotide sequence by ligating it to a heterologous sequence, thereby enabling the production of a fusion protein. Certain vectors are designed to incorporate such heterologous sequences so that insertion of a polynucleotide into the vector at an appropriate location results in an in-frame fusion to the heterologous sequence, which may be either upstream or downstream from the inserted polynucleotide. Such heterologous sequences may encode tags or cleavable linker sequences such as glutathione S-transferase (GST), the hemaglutinin epitope known as HA tag, a short stretch of the Myc protein (Myc tag), FLAG tag, 6× His tag, maltose binding protein tag, etc. In general, many of these tags are useful for the purification and/or detection of the polypeptide using an antibody or other reagent that binds to the tag. Other useful heterologous sequences include that of fluorophores such as green fluorescent protein (GFP), which allows visualization of the fusion protein, sequences that promote cellular uptake, etc. The present invention encompasses all such fusion proteins.

Thus, in general, the invention provides a polypeptide comprising a domain consisting of a VECSM polypeptide or portion thereof, and a domain consisting of a heterlogous polypeptide, wherein the VECSM polypeptide and the heterologous polypeptide form part of a single polypeptide molecule. According to certain preferred embodiments of the invention the heterologous polypeptide comprises a readily detectable moiety, a tag, or a cleavable linker. The domains may be separated by one or more intervening amino acids. The invention provides polynucleotides encoding the VECSM fusion proteins. Such polynucleotides contain a portion that encodes a VECSM polypeptide and a portion that encodes a heterologous polypeptide, in the same reading frame. The invention further provides vectors and cells comprising these VESCM fusion polynucleotides and methods of producing the VECSM fusion polynucleotides, which are essentially the same as the methods for producing VECSM polynucleotides.

The invention further provides methods of isolating, purifying, or detecting a VECSM fusion polynucleotide. The methods for isolating or purifying a VECSM fusion polynucleotide, and certain of the methods for detecting a VECSM fusion polynucleotide, comprise the step of exposing a mixture comprising a VECSM fusion polynucleotide to an antibody that specifically binds to the heterologous polypeptide. Additional steps are similar to those described for isolating, purifying, or detecting a VECSM polypeptide using antibodies that specifically bind to the VECSM polypeptide. The invention further provides methods of detecting a VECSM fusion polynucleotide, wherein the heterologous polypeptide comprises a readily detectable moiety, comprising the step of detecting the readily detectable moiety using a suitable detection means, many of which are known in the art.

VIII. Antibodies

In another aspect, the present invention provides an antibody that specifically binds to a VECSM polypeptide selected from the group consisting of VECSM Group II or Group III polypeptides. In particular, the present invention provides an antibody that specifically binds to a polypeptide encoded by a polynucleotide comprising any of the polynucleotides listed in Table 2 or Table 3.

The invention provides each of the antibodies described above individually as well as in the form of collections of two or more antibodies. In preferred embodiments of the invention the antibodies specifically bind to a polypeptide that naturally occurs in endothelial cells, in accordance with the inventors' discovery that the genes encoding the polypeptides recognized by the antibodies are differentially expressed by endothelial cells. In particularly preferred embodiments of the invention the antibodies specifically bind to a polypeptide that naturally occurs in endothelial cells and is differentially expressed in such cells. In certain preferred embodiments of the invention the endothelial cells are mammalian cells. In particularly preferred embodiments of the invention the endothelial cells are human cells. According to certain embodiments of the invention the antibodies are polyclonal antibodies, however in preferred embodiments of the invention they are monoclonal antibodies. As noted above, the term “antibodies” can include a variety of antibody derivatives and fragments, humanized antibodies, etc., provided that such derivative or fragment or humanized antibody retains the ability to bind to the antigen recognized by the full length antibody.

Generally applicable methods for producing antibodies are well known in the art and are described extensively in references cited above. It is noted that antibodies can be generated by immunizing animals (or humans) either with a full length polypeptide, a partial polypeptide, fusion protein, or peptide (which may be conjugated with another moiety to enhance immunogenicity). The specificity of the antibody will vary depending upon the particular preparation used to immunize the animal and on whether the antibody is polyclonal or monoclonal. For example, if a peptide is used the resulting antibody will bind only to the antigenic determinant represented by that peptide. It may be desirable to develop and/or select antibodies that specifically bind to particular regions of the polypeptide, e.g., the extracellular domain. Such specificity may be achieved by immunizing the animal with peptides or polypeptide fragments that correspond to that region. Alternately, a panel of monoclonal antibodies can be screened to identify those that specifically bind to the desired region. The invention therefore provides a panel of antibodies for each endothelial cell specific marker, wherein each member of the panel specifically recognizes a different antigenic determinant present in the marker.

In general, preferred antibodies will possess high affinity, e.g., a K_{d} of <200 nM, and preferably, of <100 nM for their target. According to certain embodiments of the invention preferred antibodies do not show significant reactivity with normal tissues, e.g., tissues of key importance such as heart, kidney, brain, liver, bone marrow, colon, breast, prostate, thyroid, gall bladder, lung, adrenals, muscle, nerve fibers, pancreas, skin, etc. Antibodies with low reactivity towards heart, kidney, central and peripheral nervous system tissues and liver are particularly preferred. In the context of reactivity with tissues, the term “significant reactivity”, as used herein, refers to an antibody or antibody fragment, which, when applied to a tissue of interest under conditions suitable for immunohistochemistry, will elicit either no staining or negligible staining, e.g., only a few positive cells scattered among a field of mostly negative cells. In certain embodiments of the invention the antibodies allow detection of endothelial cells when applied to a tissue sample that contains endothelial cells, i.e., the antibodies selectively bind to endothelial cells relative to their binding to other cell types present in the tissue sample.

As noted above, the clones that were initially identified as being differentially expressed in endothelial cells represent, in many cases, partial cDNAs. However, the inventive antibodies described above are not limited to antibodies that specifically bind to polypeptides encoded by the clones but rather include antibodies that specifically bind to any region of a polypeptide encoded by the full length cDNAs corresponding to the clones. In other words, the antibodies include antibodies that bind to polypeptides that are translated from RNA transcripts transcribed from the gene whose sequence includes the cDNA clone sequence, provided that such transcript includes a portion complementary to the clone. It will be appreciated that multiple different transcripts may be transcribed from certain genes. In addition, mechanisms such as alternative splicing and other types of RNA processing may generate multiple transcripts from a single initial transcript. Hence it is possible that not all transcripts transcribed from the gene corresponding to the clone will contain a region complementary to the clone, and polypeptides translated from such transcripts will therefore not contain a region encoded by the clone.

The invention provides various methods of using the antibodies described above. For example, the antibodies may be used to detect the presence and/or number of endothelial cells in a sample obtained from a subject or in vivo. They may therefore be used to determine the presence or extent of angiogenesis and/or vascularization. The antibodies may be used to perform immunohistochemical analysis, immunoblotting, ELISA assays, etc., in order to detect the polypeptide to which the antibody specifically binds. They may be used as components of antibody arrays. The antibodies may also be used for imaging studies, as described further below. In addition, the antibodies are useful as therapeutic agents.

IX Ligands

In another aspect, the invention provides ligands that specifically bind to a VECSM polypeptide. The term “ligand” may encompass any type of molecule other than antibodies as described above. Ligands may be, for example, peptides, non-immunoglobulin polypeptides, nucleic acids, protein nucleic acids (PMAs), aptamers, small molecules, etc. Ligands that specifically bind to any of the VECSM polypeptides described herein may be identified using any of a variety of approaches. For example, ligands may be identified by screening libraries, e.g., small molecule libraries. Naturally occurring or artificial (non-naturally occurring) ligands, particularly peptides or polypeptides, may be identified using a variety of approaches including, but not limited to, those known generically as two- or three-hybrid screens, the first version of which was described in Fields S. and Song O., Nature 1989 Jul 20;340(6230):245-6. Nucleic acid or modified nucleic acid ligands may be identified using, e.g., systematic evolution of ligands by exponential enrichment (SELEX) (Tuerk, C. and Gold., L, “Systematic evolution of ligands by exponential enrichment: RNA ligands to bacteriophage T4 DNA polymerase”, Science 249(4968): 505-10, 1990). See also Jellinek, D., et al., “Potent 2′-amino-2′-deoxypyrimidine RNA inhibitors of basic fibroblast growth factor”, Biochemistry, 34(36): 11363-72, 1995, describing identification of high-affinity 2′-aminopyrimidine RNA ligands to basic fibroblast growth factor (bFGF).

In general, a screen for a ligand that specifically binds to any particular VECSM polypeptide may comprise steps of contacting VECSM polypeptide with a candidate ligand under conditions in which binding can take place; and determining whether binding has occurred. Any appropriate method for detecting binding, many of which are well known in the art, may be used. One of ordinary skill in the art will be able to select an appropriate method taking into consideration, for example, whether the candidate ligand is a small molecule, peptide, nucleic acid, etc. For example, the candidate ligand may be tagged, e.g., with a radioactive molecule. The VECSM polypeptide can then be isolated, e.g., immunoprecipitated from the vessel in which the contacting has taken place, and assayed to determine whether radiolabel has been bound. This approach may be particularly appropriate for small molecules. Phage display represents another method for the identification of ligands that specifically bind to VECSM polypeptides. In addition, determination of the three-dimensional structure of a VECSM polypeptide (e.g., using nuclear magnetic resonance, X-ray crystallography, etc.) may facilitate the design of appropriate ligands.

X. Identification, Detection, and Quantification of Endothelial Cells

The invention provides various methods of identifying, detecting, and quantifying endothelial cells, based on the discovery that VECSM polynucleotides are differentially expressed in endothelial cells. For example, the invention provides a method of identifying or quantifying endothelial cells in a sample or in a subject comprising steps of: (i) providing a sample or subject; (ii) detecting or measuring expression of a VECSM polynucleotide in the sample or subject. The presence or amount of a VECSM polynucleotide may be assessed using any appropriate technique including, but not limited to, RT-PCR, Northern blotting, cDNA or oligonucleotide microarray analysis, etc.

A second method comprises steps of: (i) providing a sample or subject; (ii) detecting or measuring expression of a VECSM polypeptide in the sample or subject. The presence or amount of a VECSM polypeptide may be assessed using any appropriate technique including, but not limited to, immunohistochemistry, ELISA assays, immunoblotting, etc. These methods may be performed using the antibodies of the invention described above. The genes listed in Tables 2 and 3 may be used to define cell type specific signatures for endothelial cells for the quantification of the number, proportion, or relative proportion of endothelial cells in mixed cell populations, as described in co-pending U.S. patent application entitled “SYSTEMS AND METHODS FOR DETERMINING CELL TYPE COMPOSITION OF MIXED CELL POPULATIONS USING GENE EXPRESSION SIGNATURES”, Agilent Docket No. 10020740-1.

W. Distinguishing Endothelial Cell Types

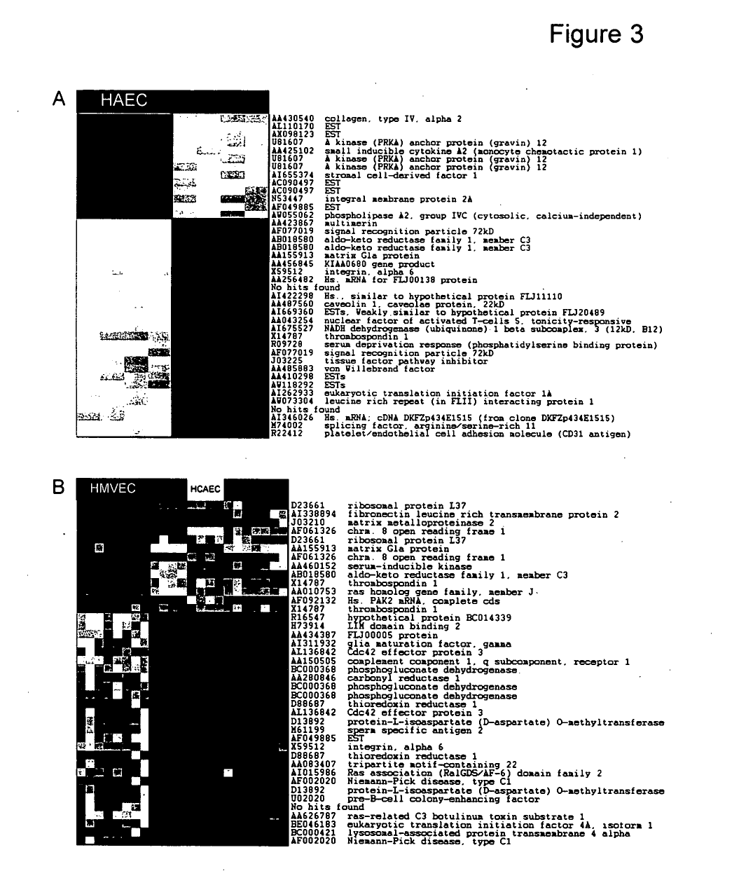

Endothelial cells have a variety of functions and properties depending on their presence within vessels of different types and sizes as well as their tissue of origin, which may be reflected by differences in gene expression (Auerbach 1985). For example, microvascular cells (i.e., cells lining capillaries) have markedly different functions from those lining conduit vessels such as arteries and arterioles. As described in Example 4, one aspect of the present invention is the identification of VECSM polynucleotides and polypeptides whose expression differs among different endothelial cell types. For example, FIG. 3B identifies a set of genes that are overexpressed in human microvascular endothelial cells relative to their expression in human aortic endothelial cells (HAEC), human coronary artery cells (HCAEC), and human umbelical vein cells (HUVEC). These genes include genes from which cDNAs identified by the following Genbank accession numbers are transcribed: R16547, H73914, AA434387, AI311932, AL136842, AA150505, BC000368, AA280846, D88687, D13892, M61199, AF049855, X59512, AA083407, A1015986, AF002020, U02020, AA626787, BE046183, and BC000421. FIG. 3B also identifies a set of genes that are underexpressed in HUVEC relative to their level of expression in HAEC, HCAEC, and HUVEC. Such genes include genes from which cDNAs identified by the following Genbank accession numbers are transcribed: D23661, AI338894, J03210, AF061326, D23661, AA155913, AF061326, AA460152, AB018580, X14787, AA010753, and AF092123.

The invention therefore provides a method of identifying an endothelial cell as a microvascular endothelial cell or of identifying a population of endothelial cells as a microvascular endothelial cell population comprising steps of: (i) measuring, in an endothelial cell or in a population of endothelial cells, the level or activity of an expression product of a gene that is overexpressed in microvascular endothelial cells relative to its expression in a different endothelial cell type; (ii) comparing the level of expression or activity of the expression product to the level of expression or activity expected in a cell or population of cells of a different endothelial cell type; and (iii) identifying the endothelial cell or population of endothelial cells as a microvascular endothelial cell or population of microvascular endothelial cells if the measured level of expression or activity is higher than would be expected in a cell or population of cells of a different endothelial cell type. In the foregoing method the gene can be any gene from which a cDNA having a Genbank accession number selected from the group consisting of accession numbers listed in Table 2 or Table 3 is transcribed.

The invention further provides a method of identifying an endothelial cell as a microvascular endothelial cell or of identifying a population of endothelial cells as a microvascular endothelial cell population comprising steps of: (i) measuring, in an endothelial cell or in a population of endothelial cells, the level or activity of an expression product of a gene that is underexpressed in microvascular endothelial cells relative to its expression in a different endothelial cell type; (ii) comparing the level of expression or activity of the expression product to the level of expression or activity expected in a cell or population of cells of a different endothelial cell type; and (iii) identifying the endothelial cell or population of endothelial cells as a microvascular endothelial cell or population of microvascular endothelial cells if the measured level of expression or activity is lower than would be expected in a cell or population of cells of a different endothelial cell type. In the foregoing method the gene can be any gene from which a cDNA having a Genbank accession number selected from the group consisting of accession numbers listed in Table 2 or Table 3 is transcribed. In any of the foregoing methods the expression product can be a polynucleotide transcribed from the gene or a polypeptide encoded by such a polynucleotide. Any of the genes identified herein as overexpressed in one or more endothelial cell types relative to the level of expression in a different endothelial cell type may be used to define a cell type specific signature for one or more endothelial cell types.

XII. Targeted Delivery Vehicles

The invention further provides a variety of delivery vehicles targeted to endothelial cells using the antibodies and/or ligands of the invention that specifically bind to VECSM polypeptides. In general, delivery vehicles are employed to improve the ability of an active molecule to achieve its desired effect on a cell, tissue, organ, subject, etc., e.g., by increasing the likelihood that the active agent will reach its site of activity. By “delivery vehicle” is meant a natural or artificial substance that is physically associated with an active molecule and provides one or more of the following functions among others: (1) conveys an active molecule within the body; (2) facilitates the uptake of an active molecule by cells, tissues, organs, etc.; (3) increases stability of an active molecule, e.g., increases half-life of the molecule; (4) changes other pharmacokinetic properties of the active molecule from what they would have been in the absence of the delivery vehicle. The active molecule may be associated with the delivery vehicle in any of a number of ways. For example, the active molecule may be bonded to the delivery vehicle (e.g., via covalent or hydrogen bonds). In certain preferred embodiments of the invention the active molecule is dispersed within or encapsulated within the delivery vehicle. By “dispersed within” is meant that individual molecules of the active molecule are intermingled with molecules comprising the material from which the delivery vehicle is made as opposed, for example, to being present as a discrete cluster of molecules.

Preferred targeting agents for use in targeting bind to a VECSM polypeptide or portion thereof that is expressed on the surface of endothelial cells. According to the invention antibodies or ligands are incorporated in and/or linked to the delivery vehicle for targeting to endothelial cells. Typically at least the portion of the antibody or ligand that binds to the VECSM polypeptide is present on the surface of the delivery vehicle, while the molecule to be delivered is typically inside. Viral vectors can be engineered to express such binding portions, peptide or polypeptide ligands, etc. Immunoliposomes (antibody-directed liposomes) can also be used. See, e.g., Bendas, G., “Immunoliposomes: a promising approach to targeting cancer therapy”, BioDrugs, 15(4), 215-24, 2001. It is noted that such targeted delivery vehicles may be used for the delivery of a wide variety of agents to endothelial cells. Typically the agent is contained within the liposome's aqueous cavity or is one of the components in its lipid membrane.

The invention further provides a targeting agent, e.g., an antibody or ligand that specifically binds to a VECSM polypeptide, wherein the targeting agent is conjugated to a support. The support can be, for example, a nanosphere, microsphere, or bead. The support can be made out of any of a variety of materials including, but not limited to, agarose, polyacrylamide, nylon, dextran, polyethylene glycol, polysaccharides such as PLA, PLGA or chitosan, other polymers, etc. Such conjugates are useful, for example, for detecting, isolating, or purifying VECSM polypeptides. These conjugates may also serve as delivery vehicles for a VECSM antibody or ligand. According to one approach, the antibodies or ligands of the invention can be conjugated to nanoparticles, which may incorporate moieties such as therapeutic agents or agents useful for imaging, as described, for example, in as described in Li, et al., J. Cell. Biochem. Suppl., 39:65-71, 2002. In addition, the invention provides targeting agents that specifically bind to VECSM polypeptides, wherein the targeting agents are conjugated to a support, and wherein an additional moiety is conjugated to the support. The additional moiety may be, for example, a therapeutic agent, an imaging agent, a readily detectable marker, an enzyme, etc.

XII. Targeting Agents Linked With a Functional Moiety

In another aspect, the invention provides compositions comprising a targeting agent linked with a functional moiety, wherein the targeting agent specifically binds to a Group II or Group III VECSM polypeptide. The invention further provides compositions comprising an targeting agent linked with a functional moiety, wherein the targeting agent specifically binds to a Group II or Group III VECSM polypeptide. Targeting agents may be any agent that specifically binds to a VECSM polypeptide. In particular, targeting agents can be antibodies or ligands that specifically bind to a VECSM polypeptide, as described above.

In general, these compositions possesses at least two functions, one of which is specifically binding to a VECSM polypeptide. The antibody may be any of the antibodies described above that bind to VECSM polypeptides. By “functional moiety” is meant any compound, agent, molecule, etc., that possesses an activity or property that alters, enhances, or otherwise changes the ability of the targeting agent to fulfill any particular purpose or that enables the targeting agent to fulfill a new purpose. Such purposes include, but are not limited to, imaging of vasculature and/or imaging of angiogenesis, providing diagnostic and/or prognostic information and/or treatment of diseases or conditions associated with inappropriate or excessive vascular endothelial growth (e.g., angiogenesis), or associated with an inadequate or reduced blood supply or inadequate or reduced angiogenesis or vascular endothelial growth, etc.

By “linked” is generally meant covalently bound or, if noncovalently bound, physically associated via intermolecular forces approximately equal in strength to that of covalent bonds. Thus a noncovalent interaction between two molecules that has very slow dissociation kinetics can function as a link. For example, an antibody is generally considered linked to its cognate antigen. As another example, reactive derivatives of phospholipids can be used to link the liposomes or cell membranes in which they are incorporated to antibodies or enzymes. Targeting agents, e.g., antibodies or ligands linked with a functional moiety will be referred to herein as conjugates or heteroconjugates. According to certain embodiments of the invention the functional moiety is a compound (e.g., polyethylene glycol) that stabilizes the targeting agent and/or increases its resistance to degradation.

According to certain embodiments of the invention the targeting agent is synthesized using precursors, e.g., amino acids, that contain the functional moiety. For example, an antibody or a polypeptide ligand can be synthesized using amino acid precursors that contain flourine-19 instead of hydrogen at one or more positions, or that contain nitrogen-15 or oxygen-17 instead of the more abundant isotope at one or more positions. As a second example, where the functional moiety is a polypeptide, the composition may be produced as a fusion protein, as described above, wherein one portion of the fusion protein (the antibody or ligand) specifically binds to the VECSM polypeptide and a second portion of the fusion protein consists of or comprises a functional moiety. Alternately, polypeptides may be modified to incorporate a functional moiety. For example, the methods described in Haruta, Y., and Seon, B. K., Proc. Nat. Acad. Sci., 83, 7898-7902 (1986) may be used to iodinate antibodies and other polypeptides. See also Tabata, M., et al., Int. J. Cancer, Vol. 82, Issue 5: 737-742, 1999. Functional moieties incorporated into a targeting agent of the invention during synthesis or added to the antibody or ligand subsequently are considered “linked” to the targeting agent.

Functional moieties may be linked to targeting agents such as antibodies by any of a number of methods that are well known in the art. Examples include, but are not limited to, the glutaraldehyde method which couples primarily through the α-amino group and ε-amino group, maleimide-sulfhydryl coupling chemistries (e.g., the maleimidobenzoyl-N-hydroxysuccinimide ester (MBS) method), and periodate oxidation methods, which specifically direct the coupling location to the Fc portion of the antibody molecule. In addition, numerous cross-linking agents are known, which may be used to link the targeting agent to the functional moiety.

A wide variety of methods (selected as appropriate taking into consideration the properties and structure of the ligand and functional moiety) may likewise be used to produce the ligand-functional moiety conjugates of the invention. Suitable cross-linking agents include, e.g., carboiimides, N-Hydroxysuccinimidyl-4-azidosalicylic acid (NHS-ASA), dimethyl pimelimidate dihydrochloride (DMP), dimethylsuberimidate (DMS), 3,3′-dithiobispropionimidate (DTBP), etc. According to certain embodiments of the invention the functional moiety is a compound (e.g., polyethylene glycol) that stabilizes the ligand and/or increases its resistance to degradation.

For additional information on conjugation methods and crosslinkers see generally the journal Bioconjugate Chemistry, published by the American Chemical Society, Columbus OH, PO Box 3337, Columbus, OH, 43210. This journal reports on advances concerning the covalent attachment of active molecules to biopolymers, surfaces, and other materials. Coverage spans conjugation of antibodies and their fragments, nucleic acids and their analogs, liposomal components, and other biologically active molecules with each other or with any molecular groups that add useful properties. Such molecular groups include small molecules, radioactive elements or compounds, polypeptides, etc. See also “Cross-Linking”, Pierce Chemical Technical Library, available at the Web site having URL www.piercenet.com and originally published in the 1994-95 Pierce Catalog and references cited therein and Wong S S, Chemistry ofProtein Conjugation and Crosslinking, CRC Press Publishers, Boca Raton, 1991. The following section presents a number of examples of specific conjugation approaches and cross-linking reagents. However, it is to be understood that the invention is not limited to these methods, and that selection of an appropriate method may require attention to the properties of the particular functional moiety, substrate, or other entity to be linked to the targeting agent.

According to certain embodiments of the invention a bifunctional crosslinking reagent is used to couple a functional moiety with a targeting agent of the invention. In general, bifunctional crosslinking reagents contain two reactive groups, thereby providing a means of covalently linking two target groups. The reactive groups in a chemical crosslinking reagent typically belong to the classes of functional groups—including succinimidyl esters, maleimides, and iodoacetamides. Bifunctional chelating agents may also be used. For example, a targeting agent of the invention may be coupled with a chelating agent, which may be used to chelate a functional moiety such as a metal. Bifunctional chelating agents may be used to couple more than one functional moiety to a targeting agent of the invention. For example, according to certain embodiments of the invention one or more of the functional moieties is useful for imaging and/or one or more of the functional moieties is useful for therapy. Appropriate chelating agents for use with the antibodies or ligands of the invention include polyaminocarboxylates, e.g., DTPA, macrocyclic polyaminocarboxylates such as 1,4,7,10-tetraazacyclododecane N,N′,N″,N′″-tetraacetic acid (DOTA), etc. See Lever, S., J. Cell. Biochem. Suppl., 39:60-64, 2002, and references therein.

The most common schemes for forming a heteroconjugate involve the indirect coupling of an amine group on one biomolecule to a thiol group on a second biomolecule, usually by a two- or three-step reaction sequence. The high reactivity of thiols and their relative rarity in most biomolecules make thiol groups good targets for controlled chemical crosslinking. If neither molecule contains a thiol group, then one or more can be introduced using one of several thiolation methods. The thiol-containing biomolecule may then be reacted with an amine-containing biomolecule using a heterobifinctional crosslinking reagent, e.g., a reagent containing both a succinimidyl ester and either a maleimide or an iodoacetamide. Amine-carboxylic acid and thiol-carboxylic acid crosslinking may also be used. For example, 1-Ethyl-3-(3-dimethylaminopropyl)carbodiimide (EDAC) can react with biomolecules to form “zero-length” crosslinks, usually within a molecule or between subunits of a protein complex. In this chemistry, the crosslinking reagent is not incorporated into the final product. The water-soluble carbodiimide EDAC crosslinks a specific amine and carboxylic acid between subunits of allophycocyanin, thereby stabilizing its assembly. See, e.g., Yeh S W, et al., “Fluorescence properties of allophycocyanin and a crosslinked allophycocyanin trimer.”, Cytometry 8, 91-95 (1987).

Several methods are available for introducing thiols into biomolecules, including the reduction of intrinsic disulfides, as well as the conversion of amine, aldehyde or carboxylic acid groups to thiol groups. Disulfide crosslinks of cystines in proteins can be reduced to cysteine residues by dithiothreitol (DTT), tris-(2-carboxyethyl)phosphine (TCEP), or or tris-(2-cyanoethyl)phosphine. Amines can be indirectly thiolated by reaction with succinimidyl 3-(2-pyridyldithio)propionate (SPDP) followed by reduction of the 3-(2-pyridyldithio)propionyl conjugate with DTT or TCEP. Amines can be indirectly thiolated by reaction with succinimidyl acetylthioacetate followed by removal of the acetyl group with 50 mM hydroxylamine or hydrazine at near-neutral pH. Tryptophan residues in thiol-free proteins can be oxidized to mercaptotryptophan residues, which can then be modified by iodoacetamides or maleimides

Reagents used to crosslink liposomes, cell membranes and potentially other lipid assemblies to biomolecules typically comprise a phospholipid derivative to anchor one end of the crosslink in the lipid layer and a reactive group at the other end to attach the membrane assembly to the target biomolecule.

For purpose of covalently linking active molecules (e.g., toxins or therapeutic agents) to targeting agents, it may be preferred to select methods that result in a conjugate wherein the targeting agent is separable from the toxin to allow the toxin to enter the cell. Thiol-cleavable, disulfide-containing conjugates may be employed for this purpose. Cells are able to break the disulfide bond in the cross-linker, which permits release of the toxin within the target cell. Examples of suitable cross-linkers include 2-Iminothiolane (Traut's reagent), N-succinimidyl 3-(2-pyridyldithio)propionate (SPDP), etc. In addition, it is generally preferable to select methods that do not significantly impair the ability of the targeting agent to specifically bind to its target and do not significantly impair the ability of the functional moiety to perform its intended function. One of ordinary skill in the art will be able to test the conjugate to determine whether the targeting agent retains binding ability and/or whether the functional moiety retains its function.

According to certain embodiments of the invention the functional moiety is released from the targeting agent upon uptake into the cell. For example, the functional moiety may be attached to the targeting agent via a linker or spacer that is cleaved by an intracellular enzyme. According to certain embodiments of the invention the finctional moiety is an antisense molecule, ribozyme, siRNA, or shRNA which may be targeted to any transcript present in endothelial cells. In general, the antibodies and ligands of the invention that specifically bind to VECSM polypeptides may be used as described in Allen, T., Nature Reviews Cancer, Vol. 2, pp. 750-765, 2002, and references therein.

According to certain embodiments of the invention the functional moiety is one that causes, either directly or indirectly, a change in the physiological (i.e., functional) and/or biochemical state of a cell with which it comes into contact. In general, a change in the physiological state of a cell will involve multiple biochemical changes. By “directly causing” is meant that the functional moiety either causes the change itself or by interacting with one or more cellular or extracellular constituents (e.g., nucleic acid, protein, lipid, carbohydrate, etc.) not introduced or induced by the hand of man. The category of direct causation includes instances in which the functional moiety initiates a “pathway”, e.g., in which the functional moiety interacts with one or more constituents, which causes a change in the interaction(s) of this constituent with other constituents, ultimately leading to the alteration in physiological or biochemical state of the cell. By “indirectly causing” is meant either (i) that the functional moiety itself does not cause the change but must be converted into an active form (e.g., by a cellular enzyme) in order to cause the change; or (ii) that the functional moiety itself does not cause the change but instead acts on a second agent that causes the change, which second agent is also introduced to or induced in the cell, its surface, or vicinity by the hand of man.

Various examples of changes in physiological or biological state include, but are not limited to, increases or decreases in gene expression (e.g., increases or decreases in transcription, translation, and/or mRNA or protein turnover), alterations in subcellular localization or secretion of a cellular constituent, alteration in cell viability or growth rate, alteration in differentiation state, etc. According to certain embodiments of the invention the functional moiety is a growth stimulatory or inhibitory agent. For example, the finctional moiety may comprise or encode a growth factor, a growth factor receptor, or an agonist or antagonist of a growth factor receptor, wherein the growth factor, growth factor receptor, growth factor receptor agonist, or growth factor receptor antagonist stimulates or inhibits growth or division of endothelial cells, and wherein presence of the growth factor receptor in or on the surface of endothelial cells may either stimulate or inhibit their growth or division depending at least in part on the presence of agonists or antagonists.

Functional moieties that stimulate growth and/or division of endothelial cells include, but are not limited to, vascular endothelial growth factor, granulocyte macrophage colony stimulating factor, angiopoietin 1 or 2, epidermal growth factor, nerve growth factor, transforming growth factor-β, tumor necrosis factor α, platelet-derived growth factor, insulin-like growth factor, acidic fibroblast growth factor, basic fibroblast growth factor, hepatocyte growth factor, brain-derived neurotrophic factor, keratinocyte growth factor, bone morphogenetic protein, or a cartilage-derived growth factor. Additional stimulatory agents include various integrins, PECAM, MMP, VE-cadh, CXC, COX2, and IL-8. Receptors whose presence in or on the surface of endothelial cells stimulates their growth and/or division in the presence of an appropriate ligand include, but are not limited to, VEGF receptors, FGF receptors, and Tie2 receptors. In addition, agonists of these growth factor receptors stimulate growth of endothelial cells. Such agonists include both endogenous ligands and also various naturally occurring or artificial ligands, including small molecules.

Functional moieties that inhibit growth and/or division of endothelial cells include, but are not limited to, angiostatin, bevacizumab, arrestin, canstatin, combretatastin, endostatin, interferon-α, NM-3, thrombospondin, tumstatin, thalidomide, 2-methoxyestradiol, vitaxin, EGF receptor, the VEGF receptor, the PDGF receptor, and ERBB-2 (HER-2/neu). Functional moieties that inhibit growth and/or division of endothelial cells also include enzymes such as stromolysin, elastase, gelatinase that cleave a precursor of an endogenous inhibitor of angiogenesis, resulting in formation of the active species. Receptors whose presence in or on the surface of endothelial cells inhibits their growth in the presence of the appropriate ligand include the EGF receptor, the VEGF receptor, the PDGF receptor, and ERBB-2 (HER-2/neu). Such ligands include both endogenous antagonists and also various naturally occurring or artificial ligands, including small molecules.

The examples given above are representative only and are not intended to be limiting. The invention encompasses the use of other moieties having the function or effect of stimulating or inhibiting growth and/or division of endothelial cells. See, e.g., Scappaticci, F., et al., J. Clin. Oncol., 20(18): 3906-3927, 2002. Whether any particular functional moiety stimulates or inhibits growth and/or division of endothelial cells may readily be tested either using in vitro tissue culture systems in which endothelial cells are contacted with the functional moiety is and their growth and/or division is then measured, or in vivo, in either animals or humans. In the latter case, the ability of the moiety to stimulate or inhibit growth and/or division of endothelial cells may be assessed using, for example, various imaging techniques (see below), or by taking samples of vascular endothelium and assessing its proliferative state (e.g., by determining the mitotic index, measuring expression or activity of proteins associated with cell division, etc.).

According to certain embodiments of the invention the functional moiety is toxic to cells. Typically, these cells will be endothelial cells to which the antibody specifically binds. However, the functional moiety may (instead or in addition) be toxic to cells in the vicinity of such endothelial cells. By “toxic to cells” is meant that the moiety either kills the cells or substantially inhibits their growth (e.g., by increasing the average doubling time of a cell population by at least a factor of 2, preferably by at least a factor of 5, more preferably by at least a factor of 10). The moiety may be directly or indirectly toxic. For example, a directly toxic moiety may interact with or inhibits one or more cellular or extracellular constituents that is required for continued cell viability, growth, or cell division in such a way as to compromise cell viability, growth, or cell division or may, by interacting with one or more cellular or extracellular constituents, trigger a pathway such as apoptosis, etc.

Alternately, the moiety may be considered indirectly toxic if it must be transformed to a different molecule in order to achieve its toxic effects (e.g., a prodrug). For example, one approach to inducing cell type specific toxicity is to deliver a relatively nontoxic molecule (prodrug) that is capable of being converted into a toxin when acted upon by an enzyme to a subject. The molecule may become widely distributed throughout the subject's body, but in the absence of the converting enzyme does not produce deleterious effects. The converting enzyme may then be targeted to specific cells that it is desired to kill or inhibit via an antibody-enzyme conjugate. This method is frequently referred to as antibody-directed enzyme prodrug therapy. Alternately, the antibody-enzyme conjugate may be delivered prior to delivery of the prodrug. See, e.g., Jung, M., “Antibody directed enzyme prodrug therapy (ADEPT) and related approaches for anticancer therapy”, Mini Rev. Med. Chem., 1(4):399-407, 2001.

Any of a variety of agents may serve as the toxic functional moiety (also referred to herein as a toxin). These include, but are not limited to, pharmaceuticals used in the treatment of cancer (chemotherapeutic agents), a large number of which are listed, for example, in the Physician's Desk Reference 2003 (published by Thomson Medical Economics, Montvale, N.J.), toxic peptides, polypeptides, or proteins (or subunits thereof) such as ricin (deglycosylated ricin A chain may be preferred), abrin, cholera toxin, Pseudomonas exotoxin, diptheria toxin, tissue factor, tumor necrosis factor, etc., certain radioactive atoms such as phosphorus-32, iodine-125, iodine-131, indium-111, rhenium-186, rhenium-188, yttrium-90, samarium-153, holmium-166, lutetium-177, promethium-149, astatine-211, etc. (or molecules containing such elements). Such atoms may be attached to antibodies or ligands via chelating agents (employing, for example, an organic chelating agent such as DTPA attached to the antibody as described in U.S. Pat. No. 4,472,509), or by direct labeling (e.g., of tyrosine residues). See also WO 02/36771 and U.S. Pat. No. 6,451,312, and references cited therein, which describe numerous cytotoxic moieties and methods of linking them to antibodies or ligands, etc. In certain preferred embodiments of the invention the isotopes and the number of radioactive elements in the functional moiety are selected so that a dose of more than 4000 cGy, e.g., 6000, 8000, or 10,000 cGy is delivered to target cells.

According to certain embodiments of the invention the functional moiety is a radiosensitizer, i.e., an agent that increases the cytotoxic effect of subsequently delivered radiation. Radiosensitizers include, e.g., molecules such as fluoropyrimidines, hydroxyurea, halogenated pyrimidines, thymidine analogs, gemcitabine, fludarabine, etc., expression products of certain genes such as p53, cyclin D, etc. According to certain embodiments of the invention the functional moiety is suitable for use in photodynamic therapy. Drugs used for this purpose include ALA, Foscan, and Photofrin. More generally, various porphyrins, chlorins, phthallocyanins, and hypocrellins may be used in various embodiments of the invention.

According to certain embodiments of the invention the functional moiety is a nucleic acid, which may serve as a template for a transcript to be expressed in the cell. The transcript may encode a polypeptide to be expressed within the cell or may act as a ribozyme, antisense molecule, siRNA, or shRNA any of which may reduce or inhibit expression of a target transcript, e.g., by cleaving the transcript (in the case of ribozymes), causing degradation of the transcript, and/or inhibiting its translation. It will be appreciated that the effect of a ribozyme, antisense molecule, siRNA, or shRNA will depend, in general, upon the particular target transcript. In certain embodiments of the invention the ribozyme, antisense molecule, siRNA, or shRNA is toxic to the cell. Suitable target transcripts for such ribozymes, antisense molecules, siRNAs, or shRNAs include transcripts whose expression product is essential for cell viability, cell division, or cell growth, including transcripts that encode anti-apoptotic polypeptides, e.g., Bcl-2. Antisense, ribozyme, siNRA, and shRNA molecules are discussed further above.

As mentioned above, recent research has shed light on the molecular mechanisms of angiogenesis (reviewed in Carmeliet P. “Mechanisms of angiogenesis and arteriogenesis”, Nat Med, 6:389-395, 2000). Manipulating angiogenic pathways, e.g, activating or inhibiting angiogenesis holds promise for the treatment and prevention of a variety of conditions associated with insufficient blood supply or excessive or inappropriate angiogenesis. For example, inhibiting angiogenesis holds promise for the prevention and/or treatment of tumors, and therapy with various anti-angiogenic agents has led to reduction in tumor volume (tumor regression) (reviewed in Carmeliet P, Jain R K. “Angiogenesis in cancer and other diseases”, Nature, 407:249-257, 2000).

XIV. Reagents and Methods for Detection and Imaging of Vasculature and Angiogenesis

As described above, the invention provides a composition comprising a targeting agent linked to a functional moiety, wherein the targeting agent specifically binds to a VECSM polypeptide. According to certain embodiments of the invention the functional moiety is a readily detectable moiety. In general, a readily detectable moiety has a property such as fluorescence, chemiluminescence, radioactivity, color, magnetic or paramagnetic properties, etc., which property renders it detectable by instruments that detect fluorescence, chemiluminescence, radioactivity, color, or magnetic resonance, etc. Alternately, a readily detectable moiety may comprise or encode an enzyme that acts on a substrate to produce a readily detectable compound. According to certain embodiments of the invention the readily detectable moiety is one that, when present at a target site subsequent to administration of the inventive composition to a subject, can be detected from outside the body. In certain preferred embodiments of the invention the readily detectable moiety can be detected non-invasively.

A variety of different moieties suitable for imaging (e.g., moieties suitable for detection by X-ray, fluoroscopy, computed tomography, magnetic resonance imaging, positron emission tomography, gamma tomography, electron spin resonance imaging, optical or fluorescence microscopy, etc.) can be used. Such agents are referred to herin as “imaging agents”. Imaging agents include, but are not limited to, radioactive, paramagnetic, or supraparamagnetic atoms (or molecules containing them). Suitable radioactive atoms include technetium-99m, thallium-211, iodine-133; atoms with magnetic moments such as iodine-123, iodine-131, indium-Ill, fluorine-19, carbon-13, nitrogen-15, oxygen-17, gadolinium, manganese, or iron. Other suitable atoms include rhenium-186 and rhenium-188. Useful paramagnetic ions include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), europium, and erbium (III), with gadolinium being particularly preferred. Gd-chelates, e.g., DTPA chelates, may be used. For example, the water soluble Gd(DTPA)²-chelate, is one of the most widely used contrast enhancement agents in experimental and clinical imaging research. The DTPA chelating ligand may be modified, e.g., by appending one or more functional groups preferably to the ethylene diamine backbone. Ions useful in other contexts, such as X-ray imaging, include but are not limited to lanthanum (III), gold (III), lead (II), and bismuth (III). Additional moieties useful for imaging include gallium-67, copper-67, yttrium-90, and astatine-211. Moieties useful for optical or fluorescent detection include fluorescein and rhodamine and their derivatives. Agents that induce both optical contrast and photosensitivity include derivatives of the phorphyrins, anthraquinones, anthrapyrazoles, perylenequinones, xanthenes, cyanines, acridines, phenoxazines and phenothiazines (Diwu, Z. J. and Lown, J. W., Pharmacology and Theraeutics 63: 1-35, 1994; Grossweiner, L. I., American Chemical Society Symposium Series 559: 255-265, 1994). Further information regarding methods and applications of molecular imaging in contexts including basic research, diagnosis, therapeutic monitoring, drug development, etc., may be found in articles appearing in the Journal of Cellular Biochemistry, Volume 87, Issue S39 (Supplement), 2002.

The readily detectable moiety may be linked to the targeting agent using various methods as described above. It is noted that many of these moieties may also be useful for therapeutic applications. See, e.g., U.S. Pat. Nos. 5,021,236 and 4,472,509, for various diagnostic agents known in the art to be useful for imaging purposes and methods for their attachment to antibodies. See also discussion above describing coupling of antibodies and ligands of the invention with functional moieties.

According to certain embodiments of the invention the functional moiety is able to bind to an additional moiety, which may impart additional functions. For example, the functional moiety may be a bispecific antibody, one portion of which binds to the VECSM polypeptide and another portion of which binds to a second molecule, e.g., another functional moiety. Alternately, a second molecule may be linked covalently to the functional moiety.

Accordingly, the invention provides a method of imaging angiogenesis, vasculature, or body tissue comprising endothelial cells in a sample or subject, comprising steps of: (i) administering to the sample or subject an effective amount of a targeting agent that specifically binds to a VECSM Group II or Group III polypeptide, wherein the targeting agent is linked to a functional moiety that enhances detectability of endothelial cells by an imaging procedure; and (ii) subjecting the sample or subject to the imaging procedure. According to certain embodiments of the invention the targeting agent specifically binds to a VECSM Group III polypeptide. The targeting agent may be, for example, an antibody or ligand that specifically binds to the polypeptide. The body tissue comprising endothelial cells may be any body tissue that contains capillaries or other blood vessels. The methods are useful for imaging both newly developing capillaries and blood vessels as well as existing vasculature and vascularized body tissues such as solid organs (e.g., heart, lung, kidney, etc.). Appropriate imaging procedures include, but are not limited to, X-ray, fluoroscopy, computed tomography, magnetic resonance imaging, positron emission tomography and variants thereof such as SPECT or CT-PET, gamma tomography, electron spin resonance imaging, optical or fluorescence microscopy.

Imaging of angiogenesis and/or vascularization has a wide range of applications. For example, the extent of angiogenesis has been correlated with survival in stroke patients (Krupinski, J., et al., Stroke, 25:1794-1798, 1994). Imaging angiogenesis in such settings may be used to provide prognostic information and/or to assess the effects of therapy.

Imaging of tumor angiogenesis, e.g., microvascular density, is useful for noninvasive characterization of tumors, distinguishing benign proliferative lesions from cancer, detecting subclinical tumors and/or metastases, monitoring effects of treatment (e.g., anti-angiogenic therapy), etc. See, e.g., Padhani, A., J. Magnetic resonance Imaging, 16:407-422, 2002; Ogawa, M., et al., Nuclear Medicine and Biology, 30: 1-9, 2003; Pearlman, J. D., et al., Current Pharmaceutical Design, 8: 1467-1496, 2002, Hoffinan, R., Lancet Oncology, 3: 546-556, 2002; Neeman, M., J. Cell. Biochem. Suppl., 39:11-17, 2002; Costouros, N., et al., J. Cell. Biochem. Supp., 39:72-78, 2002, and references in the foregoing, for a discussion of some applications of imaging angiogenesis. As described therein, current methods of imaging angiogenesis include nuclear imaging (SPECT, PET, CT-PET, micro-PET), MRI, including dynamic contrast-enhanced MRI, X-ray angiography, digital subtraction angiography, and optical imaging (e.g., intravital videomicroscopy, in vivo imaging such as whole-body imaging) using optical markers such as flourescent proteins (e.g., green fluorescent protein), bioluminescent enzymes (e.g., luciferase), and near infrared probes (e.g., Cy5.5 labeled proteins). In general, the imaging methods of the invention may be used for similar purposes. In addition, any of the optical markers may be used in conjunction with the antibodies or ligands of the invention for imaging angiogenesis.

XV. Diagnostic Applications

It will be appreciated that any of the above imaging methods may be used for to provide diagnostic and/or prognostic information related to a disease or clinical condition associated with inappropriate or excessive angiogenesis or vascular endothelial growth, or a disease or clinical condition associated with reduced or inadequate blood supply and/or to monitor the effect of therapy directed to such a disease or clinical condition. In addition, the invention provides a method of providing diagnostic or prognostic information related to a disease or condition associated with inappropriate or excessive angiogenesis or vascular endothelial growth comprising steps of: (i) providing a subject in need of diagnostic or prognostic information related to a disease or condition associated with inappropriate or excessive angiogenesis or vascular endothelial growth; and (ii) determining the level of expression or activity of a VECSM polynucleotide or polypeptide in the subject or in a biological sample obtained from the subject. According to certain embodiments of the invention, if the level of expression or activity of the VECSM polynucleotide or polypeptide is higher than would be expected in a normal subject or in a biological sample obtained from a normal subject (where by “normal subject” is meant a subject not suffering from a disease or clinical condition associated with inappropriate or excessive angiogenesis or reduced or inadequate blood supply), there is an increased likelihood that the subject is at risk of or suffering from a disease or condition associated with inappropriate or excessive angiogenesis or vascular endothelial growth. In addition, a decrease in the level or activity of a VECSM polynucleotide or polypeptide, relative to the level or activity of the polynucleotide or polypeptide when previously measured in the same subject or in a biological sample obtained from the subject, serves as an indication that a therapy designed to alleviate inappropriate angiogenesis or vascular endothelial cell growth is having the desired effect.

The invention also provides a method of providing diagnostic or prognostic information related to a disease or condition associated with reduced or inadequate blood supply comprising steps of: (i) providing a subject in need of diagnostic or prognostic information related to a disease or condition associated with inadequate or reduced blood supply or inadequate or reduced angiogenesis or vascular endothelial growth; and (ii) determining the level of expression or activity of a VECSM polynucleotide or polypeptide in the subject or in a biological sample obtained from the subject. According to certain embodiments of the invention, if the level of expression or activity of the VECSM polynucleotide or polypeptide is lower than would be expected in a normal subject or in a biological sample obtained from a normal subject (where by “normal subject” is meant a subject not suffering from a disease or clinical condition associated with inadequate or reduced blood supply or inadequate or reduced angiogenesis or vascular endothelial growth), there is an increased likelihood that the subject is at risk of or suffering from a disease or condition associated with inadequate or reduced blood supply or inadequate or reduced angiogenesis or vascular endothelial growth. In addition, an increase in the level or activity of a VECSM polynucleotide or polypeptide, relative to the level or activity of the polynucleotide or polypeptide when previously measured in the same subject or in a biological sample obtained from the subject, serves as an indication that a therapy designed to alleviate a inadequate or reduced blood supply or inadequate or reduced angiogenesis or vascular endothelial growth is having the desired effect.

In any of the foregoing methods the level of expression of an expression product (e.g., an RNA transcribed from a gene or a polypeptide encoded by such an RNA) can be determined according to standard methods, some of which are described elsewhere herein.

XVI. Therapeutic Applications and Screening Methods

A. Compositions and Methods for Inhibiting or Stimulating Angiogenesis

1. Inhibiting Angiogenesis

The invention provides a variety of compositions and methods for inhibiting or increasing angiogenesis. In particular, the invention provides a composition comprising (i) a targeting agent; and (ii) a finctional moiety, wherein the targeting agent specifically binds to a VECSM polypeptide, and wherein the functional moiety comprises an angiogenesis inhibitor. According to certain embodiments of the invention the angiogenesis inhibitor is an inhibitor of expression or activity of a pro-angiogenic protein, preferably a pro-angiogenic protein that is expressed by endothelial cells. The targeting agent may be, for example, an antibody or ligand.

The invention also provides a method of inhibiting angiogenesis in a subject comprising steps of: (i) providing a subject having a condition characterized by excessive or inappropriate angiogenesis; and (ii) administering a composition comprising (i) a targeting agent; and (ii) a functional moiety, wherein the targeting agent specifically binds to a VECSM polypeptide, and wherein the functional moiety comprises an angiogenesis inhibitor, to the subject, thereby inhibiting angiogenesis in the subject. The method may further comprise the step of determining that the subject has a condition characterized by excessive or inappropriate angiogenesis. The methods may also comprise the step of administering a second angiogenesis inhibitor, which may be present in the composition. The methods may be used for the treatment and/or prevention of any condition characterized by excessive or inappropriate angiogenesis.

A variety of angiogenesis inhibitors are known, and a number are under evaluation in clinical trials. See Kerbel, R. and Folkman, J., Nature Reviews Cancer, 2, 727-739, Oct. 2002 for a review and references therein, all of which are incorporated herein by reference, for additional information. Examples of angiogenesis inhibitors include naturally occurring peptides such as angiostatin or endostatin, precursors of an angiogenesis inhibitor (e.g., plasminogen), and a variety of small molecules. Angiogenesis inhibitors have been classified as either “direct” or “indirect”, although some inhibitors may fall into both categories. In general, direct angiogenesis inhibitors, such as vitaxin, angiostatin and others, prevent vascular endothelial cells from proliferating, migrating, or avoiding cell death in response to a spectrum of pro-angiogenic proteins, including VEGF, bFGF, IL-8, platelet-derived growth factor (PDGF) and platelet-derived endothelial growth factor (PD-EGF), pleiotrophin, etc.

Direct angiogenesis inhibitors include, but are not limited to, angiostatin, bevacizumab, arrestin, canstatin, combretatastin, endostatin, NM-3, thrombospondin, tumstatin, 2-methoxyestradiol, and vitaxin. (See Kerbel and Folkman and references cited therein). Direct inhibitors, and also certain indirect inhibitors, typically target genetically stable endothelial cells rather than unstable mutating tumor cells, which may reduce the likelihood that they will induce acquired drug resistance (Kerbel, R., Bioessays, 13, 31-36, 1991; Boehm, T., Nature, 390, 404-407, 1997.) Indirect angiogenesis inhibitors generally prevent the expression of or block the activity of a protein (typically a tumor cell protein) that activates angiogenesis, or block the expression of its receptor on endothelial cells.

Targets of indirect angiogenesis inhibitors include the EGF receptor, the VEGF receptor, the PDGF receptor, and ERBB-2 (HER-2/neu). Signaling pathways involving these receptors lead to increased expression and/or activity of a variety of pro-angiogenic proteins such as VEGF, bFGF, TGF-α, VEGF receptor, PDGF receptor, angiopoietin-1, TGF-β, and PAI1. Angiogenesis inhibitors that act as antagonists of these targets include antibodies that specifically bind to them and also a variety of small molecules. Many of the pro-angiogenic proteins are the products of oncogenes that drive or contribute to the angiogenic switch. Examples of such oncogenes include, but are not limited to, K-Ras, H-Ras, Src, c-Myb, n-Myc, c-Myc, ErbB2, EGFR, PyMT, Fos, trkB, HPV-16, v-p3k, ODC, PTTG1, E2a-Pbx1, and Bcl-2. Expression or over-expression of these proteins leads to increased angiogenesis. Any agent, whether a small molecule, antisense molecule, ribozyme, siRNA, etc., that inhibits the expression and/or activity of the expression product of any of these and other pro-angiogenic genes and oncogenes (or encodes such an inhibitor) may function as an angiogenesis inhibitor and serve as the functional moiety in the methods for inhibiting angiogenesis.

In certain embodiments of the invention the finctional moiety is or encodes an enzyme such as stromolysin, elastase, gelatinase, etc., that may convert a precursor of an angiogenesis inhibitor into active form. See Robert Kerbel and Judah Folkman, “Clinical Translation of Angiogenesis Inhibitors”, Nature Reviews Cancer 2, 727-739, 2002, for a discussion of angiogenesis inhibitors and their role in therapy. While the discussion therein focuses on cancer therapy, one of ordinary skill in the art will be able to make use of the teachings in the context of other applications of angiogenesis inhibitors.

2. Stimulating Angiogenesis

Therapeutic angiogenesis is a promising new method of treatment for patients with an impaired or inadequate blood supply. Typically impaired blood supply occurs as a result of arterial disease, e.g., coronary artery disease or peripheral vascular disease, in which vessels become occluded. The goal of this strategy is to promote the development of supplemental blood conduits that will act as endogenous bypass vessels. See Kutryk M J, Stewart D J, Microsc Res Tech, Feb 1;60(2):138-58, 20003 for a review. New vessel formation occurs through the processes of angiogenesis (which is understood to include vasculogenesis and arteriogenesis), which may take place under the influence of growth factors such as those that belong to the vascular endothelial growth factor (VEGF), fibroblast growth factor (FGF) and angiopoietin (Ang) families of molecules, which stimulate endogenous receptors such as the VEGF receptor, FGF receptor, and Tie2 receptors, which are present in or on endothelial cells.

The invention accordingly provides a method of stimulating angiogenesis in a subject comprising steps of: (i) providing a subject having a condition characterized by an inadequate blood supply to one or more organs or tissues; and (ii) administering a composition comprising (i) a targeting agent that specifically binds to a VECSM polypeptide; and (ii) a functional moiety, wherein the functional moiety comprises a stimulator of angiogenesis, to the subject, thereby stimulating angiogenesis in the subject. The method may further comprise the step of determining that the subject has a condition characterized by excessive or inappropriate angiogenesis. The method may also comprise the step of administering a second angiogenesis stimulator. The polypeptide may be a VECSM Group II or Group III polypeptide.

A number of angiogenesis stimulators are known, including vascular endothelial growth factor, granulocyte macrophage colony stimulating factor, angiopoietin 1 or 2, epidermal growth factor, nerve growth factor, transforming growth factor-β, tumor necrosis factor α, platelet-derived growth factor, insulin-like growth factor, acidic fibroblast growth factor, basic fibroblast growth factor, hepatocyte growth factor, brain-derived neurotrophic factor, keratinocyte growth factor, bone morphogenetic protein, or a cartilage-derived growth factor. Additional agents include various integrins, PECAM, MMP, VE-cadh, CXC, COX2, and IL-8. Any of these, or portions thereof, may serve as the functional moiety in the methods for stimulating angiogenesis.

In any of the methods above, where the functional moiety is a receptor, e.g., a growth factor receptor, the invention contemplates administration of a composition comprising one or more agonists or antagonists of the receptor, either in isolated form or linked to an antibody or ligand that specifically binds to a VECSM polypeptide. Preferably the composition comprising one or more agonists or antagonists of the receptor is administered either shortly before, at the same time as, or after administration of the composition comprising the receptor.

B. Compositions and Methods for Inhibiting Endothelial Cell or Tumor Growth or Survival

In addition to compositions and methods for inhibiting angiogenesis (discussed above) the invention provides compositions and methods for killing or inhibiting the growth or survival of endothelial cells. These compositions and methods may be used to inhibit angiogenesis and/or to destroy existing vasculature. The invention provides a composition comprising (i) a targeting agent that specifically binds to a VECSM polypeptide; and (ii) a functional moiety, wherein the functional moiety comprises a cytotoxic agent. The invention further provides a method of killing or inhibiting the growth or survival of endothelial cells comprising steps of: contacting the endothelial cells with a composition comprising (i) a targeting agent that specifically binds to a VECSM polypeptide; and (ii) a functional moiety, wherein the functional moiety comprises a cytotoxic agent, thereby killing or inhibiting growth or survival of the endothelial cells.

The invention further provides a method of treating a subject comprising steps of: (i) providing a subject suffering from a condition characterized by excessive or inappropriate angiogenesis or vasculature; and administering to the subject a composition comprising (i) a targeting agent that specifically binds to a VECSM polypeptide; and (ii) a functional moiety, wherein the functional moiety comprises a cytotoxic agent, thereby killing or inhibiting growth or survival of endothelial cells. The VECSM polypeptide can be a Group II or Group III polypeptide.

The invention further provides a method of inhibiting tumor growth or survival comprising steps of: (i) providing a subject having a tumor; and (ii) administering to the subject a composition comprising (i) a targeting agent; and (ii) a functional moiety, wherein the targeting agent specifically binds to a VECSM polypeptide, and wherein the functional moiety comprises an angiogenesis inhibitor, to the subject, thereby inhibiting growth, survival, or metastasis of the tumor. The method may further comprise the step of: administering a second anti-tumor agent. The targeting agents in the foregoing methods may be antibodies or ligands that specifically bind to VECSM polypeptides. In certain embodiments of the invention the targeting agent and the functional moiety are physically linked, e.g., via a covalent or non-covalent bond.

C. VECSMPolypeptides as Therapeutic Targets

As discussed above, the discovery that VECSM polynucleotides are differentially expressed in endothelial cells suggests that they are appropriate targets for treatment or prevention of diseases and clinical conditions associated with inappropriate or excessive vascular endothelial growth (including, but are not limited to, cancer, atherosclerosis, restenosis, psoriasis, rheumatoid arthritis, diabetic retinopathy, endometriosis, menorrhagia, hemangiomas, and vascular malformations) and/or diseases and clinical conditions may associated with a reduced or inadequate blood supply (including, but not limited to, ischemic cardiovascular diseases, diabetes, peripheral arterial disease, etc.). Thus the invention provides a method for treating a disease or clinical condition associated with inappropriate or excessive vascular endothelial growth comprising: (i) providing a subject at risk of or suffering from a disease or clinical condition associated with inappropriate or excessive angiogenesis or vascular endothelial growth; and (ii) administering a compound that modulates activity or abundance of a Group II or Group III VECSM polynucleotide or polypeptide to the subject. By “modulate” is meant to enhance or reduce the level or activity of a molecule or to alter the temporal or spatial pattern of its expression or activity.

The invention further provides a method for treating a disease or clinical condition associated with inadequate or reduced blood supply comprising: (i) providing a subject at risk of or suffering from a disease or clinical condition associated with a reduced or inadequate blood supply; and (ii) administering a compound that modulates activity or abundance of a Group II or Group III VECSM polynucleotide or polypeptide to the subject. According to certain embodiments of the invention VECSM polynucleotide encodes a protein with an extracellular portion, or the VECSM polypeptide comprises an extracellular portion.

D. Screening Methods

The invention encompasses methods for screening compounds for preventing or treating a disease or clinical condition associated with inappropriate or excessive angiogenesis or inadequate blood supply by assaying the ability of the compounds to modulate the expression of the endothelial-cell specific genes disclosed herein or to modulate the activity of the protein products of these genes. Appropriate screening methods include, but are not limited to, assays for identifying compounds and other substances that interact with (e.g., bind to) the target gene protein products.

Methods for identifying compounds capable of modulating gene expression are described, for example, in U.S. Pat. No. 5,976,793. The screening methods described therein are particularly appropriate for identifying compounds that do not naturally occur within cells and that modulate the expression of genes of interest whose expression is associated with a defined physiological or pathological effect within a multicellular organism.

The invention encompasses methods for identifying compounds that modulate the expression or activity of a VECSM polynucleotide or polypeptide. By “modulate” is meant increase, decrease, or otherwise alter (e.g., change the temporal or spatial expression, cell type specificity, or modification state). No particular amount of increase, decrease, or alteration is implied. In particular, the invention encompasses methods for identifying compounds that modulate the activity of a VECSM polynucleotide or polypeptide listed in Table 2 or Table 3. Methods of screening for such compounds are well known in the art and depend, to a certain degree, on the particular properties and activities of the polypeptide. In addition, the invention provides compounds that modulate expression or activity of a VECSM polynucleotide or polypeptide.

Expression of a VECSM polynucleotide or polypeptide can be measured using a variety of methods well known in the art, in general, any measurement technique capable of determining RNA or protein presence or abundance may be used for these purposes. For RNA such techniques include, but are not limited to, microarray analysis (For information relating to microarrays and also RNA amplification and labeling techniques, which may also be used in conjunction with other methods for RNA detection, see, e.g., Lipshutz, R., et al., Nat Genet., 21(1 Suppl):20-4, 1999; Kricka L., Ann. Clin. Biochem., 39(2), pp. 114-129; Schweitzer, B. and Kingsmore, S., Curr Opin Biotechnol 2001 Feb;12(1):21-7; Vineet, G., et al., Nucleic Acids Research, 2003, Vol. 31, No. 4.; Cheung, V., et al., Nature Genetics Supplement, 21:15-19, 1999; Methods Enzymol, 303:179-205, 1999; Methods Enzymol, 306: 3-18, 1999; M. Schena (ed.), DNA Microarrays: A Practical Approach, Oxford University Press, Oxford, UK, 1999. See als U.S. Pat. Nos. 5,242,974; 5,384,261; 5,405,783; 5,412,087; 5,424,186; 5,429,807; 5,436,327; 5,445,934; 5,472,672; 5,527,681; 5,529,756; 5,545,531; 5,554,501; 5,556,752; 5,561,071; 5,599,695; 5,624,711; 5,639,603; 5,658,734; 6,235,483; WO 93/17126; WO 95/11995; WO 95/35505; EP 742 287; EP 799 897; U.S. Pat. Nos. 5,514,545; 5,545,522; 5,716,785; 5,932,451; 6,132,997; 6,235,483; US Patent Application Publication 20020110827)

Other methods for detecting expression of VECSM polynucleotides include Northern blots, RNAse protection assays, reverse transcription (RT)-PCR assays, real time RT-PCR (e.g., Taqman™ assay, Applied Biosystems), SAGE (Velculescu et al. Science, vol. 270, pp. 484-487, Oct. 1995), Invader® technology (Third Wave Technologies), etc. See, e.g., Eis, P. S. et al., Nat. Biotechnol. 19:673 (2001); Berggren, W. T. et al., Anal. Chem. 74:1745 (2002), etc. Methods for detecting VECSM polypeptides include, but are not limited to, immunoblots (Western blots), immunofluorescence, flow cytometry (e.g., using appropriate antibodies), mass spectrometry, and protein microarrays (Elia, G., Trends Biotechnol 2002 Dec;20(12 Suppl):S19-22, and reference therein).

Certain of the methods may be performed in vitro, e.g., using a VECSM polypeptide or significantly similar polypeptide or fragment thereof produced using recombinant DNA technology. Certain of the methods may be performed by applying the test compound to a cell that expresses the polypeptide and measuring the expression or activity of the polypeptide, which may involve isolating the polypeptide from the cell and subsequently measuring its amount and/or activity. In certain of the methods the polypeptide may be a variant that includes a tag (e.g., an HA tag, 6× His tag, Flag tag, etc.) which may be used, for example, to facilitate isolation, or the variant may be a fusion protein.

Compounds suitable for screening according to the above methods include small molecules, natural products, peptides, nucleic acids, etc. Sources for compounds include natural product extracts, collections of synthetic compounds, and compound libraries generated by combinatorial chemistry. Libraries of compounds are well known in the art. One representative example is known as DIVERSet™, available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan, et al., “Stereoselective Synthesis of Over Two Million Compounds Having Structural Features Both Reminiscent of Natural Products and Compatible with Miniaturized Cell-Based Assays”, Am. Chem Soc. 120, 8565-8566, 1998; Floyd C D, Leblanc C, Whittaker M, Prog Med Chem 36:91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc. In certain embodiments of the invention the methods are performed in a high-throughput format using techniques that are well known in the art, e.g., in multiwell plates, using robotics for sample preparation and dispensing, etc. Representative examples of various screening methods may be found, for example, in U.S. Pat. No. 5,985,829, U.S. Pat. No. 5,726,025, U.S. Pat. No. 5,972,621, and U.S. Pat. No. 6,015,692. The skilled practitioner will readily be able to modify and adapt these methods as appropriate.

Thus the invention provides a method for screening a compound comprising steps of: (i) providing a sample comprising cells that express a VECSM Group II or Group III polypeptide; (ii) contacting the cells with the compound; and (iii) determining whether the level of expression or activity of the polynucleotide or polypeptide in the presence of the compound is increased or decreased relative to the level of expression or activity of the polynucleotide or polypeptide in the absence of the compound; and (iv) identifying the compound as a modulator of the VECSM polynucleotide or polypeptide if the level of expression or activity of the VECSM polynucleotide or polypeptide is higher or lower in the presence of the compound relative to its level of expression or activity in the absence of the compound. According to certain embodiments of the invention the VECSM polynucleotide or polypeptide is a Group III polynucleotide or polypeptide.

The invention includes compounds identified using the above methods, e.g., compounds that increase or decrease expression or increase or decrease one or more activities of a VECSM Group II or Group III polypeptide. The invention encompasses methods for treating diseases or conditions associated with excessive, inadequate, or inappropriate angiogenesis by administering compounds and other substances that modulate the overall activity of the target gene products, e.g., compounds identified using the inventive screening methods described above. Compounds and other substances can effect such modulation either on the level of target gene expression or target protein activity.

XVII. Pharmaceutical Compositions

The invention provides a variety of pharmaceutical compositions. For example, the invention provides a pharmaceutical composition comprising an effective amount of an antibody that specifically binds to a Group II or Group III VECSM polypeptide and a pharmaceutically acceptable carrier. The invention further provides a pharmaceutical composition comprising an effective amount of an antibody that specifically binds to a VECSM Group II or Group III polypeptide. The invention further provides a pharmaceutical composition comprising an effective amount of a ligand that specifically binds to a Group II or Group III VECSM polypeptide, and a pharmaceutically acceptable carrier. The antibodies and ligands may be conjugated with any of the therapeutic moieties discussed above. The invention further provides a pharmaceutical composition comprising an effective amount of an antisense molecule, ribozyme, siRNA, shRNA, or vector that provides a template for transcription of an siRNA or shRNA, wherein the antisense molecule, ribozyme, siRNA, shRNA, or vector that provides a template for transcription of an siRNA or shRNA reduces expression of a VECSM Group II or Group III polypeptide.

Compositions containing antibodies, ligands, conjugates, antisense nucleic acids, siRNA, shRNAs, ribozymes, and/or small molecules or other therapeutic agents as described herein may be formulated for delivery by any available route including, but not limited to parenteral (e.g., intravenous), intradermal, subcutaneous, oral (e.g., inhalation), transdermal (topical), transmucosal, rectal, and vaginal. Preferred routes of delivery include parenteral, transmucosal, rectal, and vaginal. Inventive pharmaceutical compositions typically include one or more therapeutic agents, in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. Compositions can also be delivered directly to a site of tissue injury or surgery. They may be administered by catheter or using diagnostic/therapeutic equipment such as bronchoscopes, colonoscopes, etc. Inventive compositions may also be delivered as implants or components of implantable devices. For example, inventive compositions may be used to coat stents and/or vascular grafts.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use typically include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringability exists. Preferred pharmaceutical formulations are stable under the conditions of manufacture and storage and must be preserved against the contaminating action of microorganisms such as bacteria and fungi. In general, the relevant carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

For administration by inhalation, the inventive therapeutic agents are preferably delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

A therapeutically effective amount of a pharmaceutical composition typically ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The pharmaceutical composition can be administered at various intervals and over different periods of time as required, e.g., one time per week for between about 1 to 10 weeks, between 2 to 8 weeks, between about 3 to 7 weeks, about 4, 5, or 6 weeks, etc. For certain conditions it may be necessary to administer the therapeutic composition on an indefinite basis to keep the disease under control. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject with a therapeutic agent as described herein, can include a single treatment or, in many cases, can include a series of treatments.

Exemplary doses include milligram or microgram amounts of the inventive therapeutic agent per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.) It is furthermore understood that appropriate doses of a therapeutic agent depend upon the potency of the agent, and may optionally be tailored to the particular recipient, for example, through administration of increasing doses until a preselected desired response is achieved. It is understood that the specific dose level for any particular animal subject may depend upon a variety of factors including the activity of the specific compound employed, the age, body weight, general health, gender, and diet of the subject, the time of administration, the route of administration, the rate of excretion, any drug combination, and the degree of expression or activity to be modulated.

Inventive pharmaceutical compositions can be included in a container, pack, or dispenser together with instructions for administration.

### EXEMPLIFICATION

### Example 1

### Identification of Candidate Endothelial Cell Specific Genes Using Database Mining

Overview. Three publicly available gene expression databases were identified and used for this analysis, Unigene at URL www.ncbi.nlm.nih.gov/SAGE/sagexpsetup.cgi, SAGE at URL www.ncbi.nlm.nih.gov/SAGE/SAGEtag.cgi and BodyMap at URL bodymap.ims.u-tokyo.acjp/gene_ranking.php. A metric was employed with each database to identify those SAGE tags that were sequenced in endothelial cell libraries and not in other cell type libraries. Unigene IDs were recorded for each gene identified, or obtained by searching the appropriate database for assignment of a Unigene number. At Unigene, it was possible to employ the Library Differential Display feature, and to identify 29 genes that were differentially expressed in cultured endothelial cells. The SAGE site had information from HMVEC cells in the presence or absence of vascular endothelial cell growth factor, and 256 genes were identified as specifically expressed in these cells. Expression data at BodyMap at the University of Tokyo, allowed comparison of human aortic intimal tissue to other non-vascular tissue types (Hishiki et al. 2000; Kawamoto et al. 2000). The intima in normal blood vessel wall is primarily endothelial cells and extracellular matrix, so this comparison was appropriate. Using BodyMap data, 200 differentially expressed genes were initially identified, and Unigene numbers obtained. IMAGE clones representing the identified Unigene clusters were ordered from Research Genetics, Inc. (See Examples 3 and 4)

Database screening methodology. Top “endothelial-specific” candidate genes were compiled from the Unigene, Serial Analysis of Gene Expression (SAGE), and BodyMap libraries as follows. Using the Library Differential Display feature of Unigene at the web site having URLwww.ncbi.nlm.nih.gov/lUniGene/ddd.cgi?ORG=Hs, a list of the top 100 genes was generated representing those differentially expressed between endothelial cell lines (pool A) and all non-vascular cell lines (pool B). A metric was developed termed the score; SCORE=Pool A/(Pool B+0.00001), with the (0.00001) factor added to prevent division by zero and to compensate for undue inflation of the score that division by a small fraction would create. The genes were then sorted by the highest SCORE, with all genes already identified with a Unigene ID. Only those genes with a SCORE greater than 10.4 were used in the final compilation of genes (a total of 29 genes).

The SAGE (Serial Analysis of Gene Expression) database at the NCBI having URL www.ncbi.nlm.nih.gov/SAGE/sagexpsetup.cgiwas queried as follows. The xProfiler tool of SAGE was used to generate two pools of cell lines to perform a virtual subtraction between endothelial and all non-endothelial cell types. In pool A, only HMVEC and HMVEC+VGEF cell lines were selected. In pool B, as many non-endothelial cell lines as possible were selected. The subtraction was performed with a factor difference of 2.0 and coefficients of variance cutoffs of 0%. From this, a list of 65,505 SAGE tags was generated and downloaded, along with the number of “hits” in pool A and pool B. This list was then resorted based upon the highest ratio of pool A to pool B (with zero hits in pool B changed to 0.9 to prevent division by zero). A score metric was devised whereby the SCORE=Group A/Group B and all genes were then sorted by this value. In some instances, a gene with a high SCORE was only identified by the SAGE tag, without information on the Unigene ID or gene name. In such cases, a search using the SAGE tag to gene mapper at the web site having URL www.ncbi.nlm.nih.gov/SAGE/SAGEtag.cgi was performed to identify the likely gene or EST represented by the tag. Genes with a SCORE of 2.2222 or greater were constituted an initial compilation of genes (a total of 368 genes). However, of these 368, no reliable matches for a Unigene cluster were found based on the SAGE tag information for 112, leaving 256 to be included in the final compilation.

Putative endothelial genes were also identified from the BodyMap gene expression database at the web site having URL bodymap.ims.u-tokyo.acjp/gene_ranking.php bodymap.ims.u-tokyo.acjp/gene_ranking.php (Hishiki et al. 2000; Kawamoto et al. 2000). The Gene Ranking System was utilized to select for genes preferentially expressed in the intima layer of the human aorta. This generated a list of 200 genes along with the number of times each gene is present in aortic intima versus other tissues (SCORE=# intima aorta/(TOTAL−intima aorta+0.0001)), and the genes were ranked based upon the highest ratio. After the genes were sorted by this score, a Unigene number was found for the top 188 genes, when possible, based on either the gene name, gene sequence, or some combination thereof. Of these 188 genes, no significant matches were found for only 10 genes, leaving 178 Bodymap genes used in the final compilation.

The top genes from each virtual subtraction were compiled (178 from BodyMap, 29 from Unigene, and 256 from SAGE for a total of 463 genes). Two of these 463 genes were later found to best match genes from the mouse and rat and were therefore excluded. Of these 461 genes, 384 genes (Unigene numbers) were submitted to Research Genetics cMiner at the web site having URL www.resgen.com/resources/apps/cminer/program and IMAGE clones obtained. The 384 genes represented by these IMAGE clones were considered promising candidate endothelial cell specific genes and were subjected to further analysis as described in Examples 3 and 4.

### Example 2

### Identification of Candidate Endothelial Cell Specific Genes Using Suppression Subtractive Hybridization

Materials and Methods

Cell Culture. Human umbilical vein endothelial cells (HUVEC), human aortic endothelial cells (HAEC), human coronary artery endothelial cells (HCAEC), human lung microvascular endothelial cells (HMVEC), human aortic smooth muscle cells (HASMC), human mammary epithelial cells (HMEC), human aortic smooth muscle cells (HASMC), normal human astrocytes (NHA), and normal human epidermal keratinocytes (NHK) were primary cultured cells obtained from Clonetics, Inc. (San Diego, Calif.). All primary human cells were from single donors ages 33 to 54, except NHA (donor 18 weeks), HASMC and pulmonary HMVEC (donor 3 years), and HUVEC (donated at birth). HCAEC and HAEC were from male donors, other cells were from females, or the gender of the source was not known. Cells were employed for studies at passage 3-9. HepG2 (human hepatocellular carcinoma cell line) cells were obtained from American Type Culture Collection (Manassas, Va.). All four endothelial cell types were plated on 100 mm culture dishes precoated with 2% gelatin (Sigma, St. Louis, Mo.) and cultured in M199 containing 15% FBS (HyClone, Logan, Utah), endothelial cell growth supplement (20 ug/ml, Sigma), heparin (20 units/ml, Sigma), glutamine, and penicillin. Non-endothelial cell types were grown in media recommended by the supplier.

Cloning by suppression subtractive hybridization. HAEC (passage 5) were serum starved for 24 hours, then treated with 3 ng/ml human recombinant TGF-β1 (R&D Systems, Minneapolis, Minn.), and harvested at 30 minute, 5 hour and 24 hour intervals. RNA isolated from these harvested cells were pooled. A polymerase chain reaction-based cDNA subtraction and normalization methodology was employed using reagents supplied in the polymerase chain reaction Select cDNA Subtraction kit (Clontech, Palo Alto, Calif.) to identify genes preferentially expressed in TGF-β stimulated endothelial cells (Diatchenko et al. 1996). Tester DNA was derived from 2 μg of TGF-β stimulated (3 ng/ml) HUVEC poly(A)+RNA, and driver DNA was derived from 2 μg of poly(A)+RNA from growth-arrested HUVEC. The reverse subtraction was performed by reversing the tester and driver. Subtraction hybridization was performed according to the manufacturer's instructions, and the 288 products of secondary polymerase chain reaction were cloned into plasmid vectors and evaluated by nucleotide sequence analysis. Clones were identified by employing the DNA sequence in automated BLAST searches of the NCBI nucleotide databases.

Results

We complemented the in silico approach described in Example 1 to identify potential endothelial cell-specific clones by performing subtraction suppression hybridization on endothelial cells stimulated with TGF-β, a known modulator of angiogenic vascular formation (Pepper 1997). From this library, 288 TGF-β, regulated endothelial cell genes were isolated and sequenced, identified through sequence database searches, and included in the construction of the custom spotted cDNA array. Accession numbers for these clones are available at the Web site having URL quertermous.stanford.edu. The 288 genes represented by these nucleotide sequences were considered promising candidate endothelial cell specific genes and were subjected to further analysis as described in Examples 3 and 4.

### Example 3

### Refinement of Candidate Endothelial Cell Specific Gene Sets

This example describes the process by which a subset of the candidate genes identified as described in Examples 1 and 2 was selected as being endothelial cell specific, with at least a three-fold difference in expression in endothelial cells relative to a mixed pool of non-endothelial cells.

Microarray construction and hybridization. For cDNA probe preparation, the 384 virtual subtraction and 288 subtraction hybridization clones were amplified by PCR employing flanking sequences of cloning vectors, according to standard methodology. Five μl of PCR reaction were visualized on 1% agarose gels for quality determination. PCR reactions were purified on a Qiagen BioRobot 3000. In addition, the 288 subtraction hybridization clones were PCR amplified and purified by both Microcon-96 filtrate (Millipore) and Arraylt kits (TeleChem International, Sunnyvale, Calif.). There were not appreciable differences in microarray results from PCR products purified by the different methods, however results are reported separately for the three different preps such that there are 1248 probes representing 672 clones. DNA microarrays were printed on glass slides employing Agilent's SurePrint inkjet technology (Agilent Technologies, Inc., Palo Alto, Calif.). The microarrays contain 6 repeat features for each of the 1248 probes. For a description of the performance features of Agilent's deposition cDNA microarrays with respect to uniformity, sensitivity, precision, and accuracy in gene expression profiling assays, see the Web site having URL www.chem.agilent.com/scripts/LiteraturePDF.asp?iWHID=27667.

Sample labeling and hybridization to the arrays was performed as follows. Ten μg of total RNA from cultured cells were reverse-transcribed in the presence of 400 units of Superscript II RNase H⁻ Reverse Transcriptase (Invitrogen, Carlsbad, Calif.), 25 μM of dCTP and 100 μM each of dATP, dTTP and dGTP, 25 μM of Cy3- or Cy5-dCTP (NEN Life Science, Boston, Mass.), 4 μM of 5′-T16N-2′ DNA primer and 27 units of Rnase inhibitor (Amersham, Piscataway, N.J.). The labeling was carried out at 42° C. for 1 hour. After degradation of unlableled RNA by RNase I, labeled cDNAs were purified with a Qiagen PCR cleanup kit. Microarray hybridization was performed at 65° C. overnight in a 25 μl of hybridization solution containing Agilent's deposition hybridization buffer, 5 units of PolydA₄₀₋₆₀, (Amersham), 5 μg of yeast tRNA (Sigma), 10 μg of human Cot 1 DNA (Invitrogen) and cy3- and cy5-prelabeled HCV deposition control targets (Qiagen Operon, Valencia, Calif.). At the end of hybridization, microarrays were first washed in 0.5×SSC/0.01% SDS for 5 min. at room temperature, then washed in 0.06×SSC wash buffer for 10 min. Finally, microarrays were dried by centrifugation. At least two separate cultures of each cell type were employed for RNA preparation and hybridization. HUVEC RNA served as a common reference for all of these experiments. At least four hybridizations were performed for each cell type, and dye reversal experiments were conducted for most cell type experiments.

Scanning, background subtraction and normalization of array data. The microarrays were scanned on an Agilent G2565AA Microarray Scanner System and the images were quantified using Agilent's G2567AA Feature Extraction Software Version A.5.0.92. Background subtraction and normalization methods not included in the commercial software were applied for this data set. For background subtraction, the array was divided into 30 localized regions, and for each region the average of the weakest 5% of features was subtracted from the raw signal for each feature. Given that the microarrays used in this study were highly enriched for genes known to be expressed in endothelial cells, it seemed likely that standard two-color DNA microarray normalization methods which rely on the assumption that the distributions of signals for both the samples in the red and green channels are identical, would not be appropriate. As the endothelial cell types tended to have higher expression for most of the probes on arrays than the non-endothelial cell types, we expected the samples to have overall biases of up and down regulation of genes. Therefore, we developed a new normalization method, which selected a set of normalization probes that were least regulated across the set of all arrays in this study. In this new method, each probe is scored by the length of the 2N-dimensional Longest Order-Preserving Sequence (LOPS) of which it is a member. Probes within the these longest (or almost longest) order preserving sequences tend to be the most “housekeeping-like”, since their rankings within each sample tend to be conserved across all the samples. Once the probes were scored, the 174 least regulated probes, corresponding to 1044 features (6 replicates per probe) on each array, were used for normalization. The normalization probe signals were fitted to a straight line in log(red signal) versus log (green signal) space, and these fit parameters were applied to calculate normalized signals for all features. Saturated, non-uniform, and population-outlier features were omitted from the normalization sets on an array-by-array basis. Signals from the six replicate features for each probe printed on the arrays were combined by averaging normalized feature signals, while eliminating the same bad or outlier features.

Data analysis. Several statistical methods were employed to identify those genes that were likely to be differentially expressed between the different cell types under consideration. SAM (Significance Analysis of Microarrays) is a widely used variant of permutation analysis developed specifically for microarrays. The algorithm employs replicate experiments to develop a measure of variance that is then used to test whether observed differences in gene expression, in two cell-type partitions, are likely to be real. SAM is described on the Web site having URL www-stat.stanford.edu/˜tibs/SAM/>(Tusher et al. 2001). The Web site also describes how to obtain a copy of the program. The SAM algorithm employed was executed by the SAM Isolator program (X-mine, Inc., Brisbane, Calif.). SAM also computes the False Discovery Rate (FDR) for any set of genes defined by how sharply they individually separate two cell-type classes. The FDR calculation uses a pseudo-random permutation process and the actual numbers assume sample independence. While this assumption is valid when two homogenous classes are considered it is not easily justified when different numbers of replicates represent different subclasses. Thus this analysis yields strong results in two way classifications such as in FIG. 3.

Parametric and non-parametric (distribution free) scoring methods described more fully at the Web site having URLwww.labs.agilent.com/resources/techreports.html were also used to identify differentially expressed genes and assess the significance of the observed differences. Some of these methods are applicable for data with more than two classes, as described below. Parametric methods assume a certain distribution for expression values of every gene within each given class (e.g., cell type) and then score genes according to how separate the class specific distributions are. The parametric method employed here was the Gaussian Error score (discussed below). Distribution free scores, in contrast, are not based on parametric assumptions. These include the Kolmogorov-Smimov score, and the Wilcoxon rank-sum test (Chakravarti and Roy 1967; Hollander and Wolfe 1973). The use of non-parametric scores for microarray data analysis have been described, e.g., in (Ben-Dor et al. 2000; Ben-Dor et al. 2001). The Threshold-Number-of-Misclassifications (TNoM) score was applied to the current data. The exact p-values for the TNoM score could be computed using combinatorial approaches with the underlying assumption of independence of samples within one class (Ben-Dor et al. 2001). This assumption is not valid for the set of experiments we consider since we include replicate experiments for the same cell type.

The Gaussian error score (GER) is applicable in the general multi-class case (such as the comparison of HAEC vs HCAEC vs HMVEC), where we seek genes that separate all classes from each other. In this 3-way comparison, the expression patterns of single genes are shown for the replicate experiments with HAEC (red), HCAEC (green), and HMVEC (blue). For each cell type a Gaussian curve (shown in the figures with the respective class color) is fit to the data. Intuitively, the more separate the three distributions. are, the more relevant the gene for the distinction between the different cell types. To formalize this intuition we compute, for each sample, t, of class (cell type) Γ(t) its error score,

$${Err}_{g} ( t ) = 1 - \frac{p ( {e}_{g} ( t ) \vert {μ}_{Γ ( t )} , {σ}_{Γ ( t )} ) p ( Γ ( t ) )}{{\sum }_{Γ} p ( {e}_{g} ( t ) \vert {μ}_{Γ} , {σ}_{Γ} ) p ( Γ )}$$

where e_{g}(t) is the expression level of the gene g in sample t and p(Γ) is the prior probability of the class Γ. The Gaussian Error score of g is then defined by

$$GER ( g ) = {\sum }_{j = 1}^{m} {Err}_{g} ( {t}_{j} ) .$$

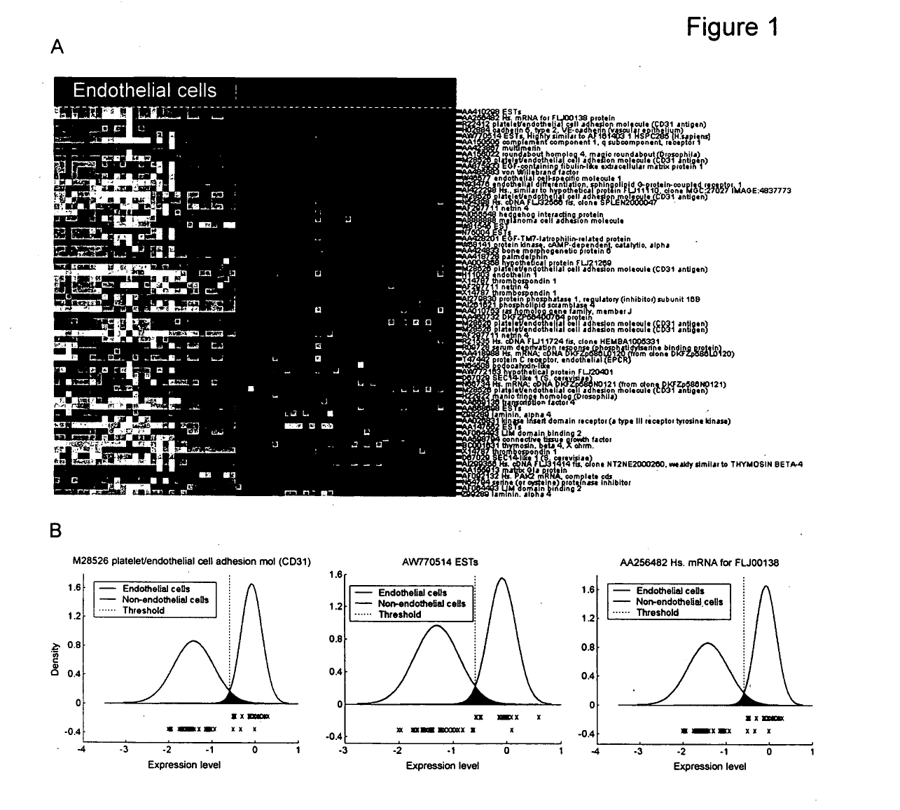

One disadvantage of parametric scores is their sensitivity to the effects of outliers. Very large or small ratio values can push the distributions apart and yield a score much better than reality supports. This can be overcome by rejecting outliers in calculating the Gaussian fits (both in the t-test and in GER). Another disadvantage is that parametric scores are usually based on homogeneous distributions within classes. This is not the case in some of these analyses, e.g., comparison of endothelial to non-endothelial cells as in FIG. 1. These problems can be avoided by using distribution-free combinatorial scores, like TNoM.

Results

Overview. Genes identified through database mining and subtraction cloning as described in Examples 1 and 2 were spotted on treated glass slides with ink jet printer methodology. These experiments involved hybridizations to microarrays derived from two separate printings. Hybridization to these arrays with ten micrograms total RNA gave consistent and reproducible results between arrays. At least four hybridizations were performed per RNA sample, and dye reversal experiments were performed for almost every RNA, providing information regarding reproducibility of the data. Primary cultured human endothelial cells derived from the umbilical vein (HUVEC), coronary artery (HCAEC), aorta (HAEC), and pulmonary microcirculation (HMVEC) were employed as models of endothelial cell lineage. Primary cultured human astrocytes (NHA), keratinocytes (NHK), mammary epithelial cells (HMEC), and aortic smooth muscle cells (HASMC), as well as the HepG2 hepatic cell tumor line, were employed as models of non-endothelial cell lineage and were compared with the endothelial cell types. Thirty hybridizations were performed with endothelial cell RNAs, and thirty-six hybridizations with non-endothelial cell RNAs. Data resulting from hybridization of RNA samples isolated from all of the cultured cell types was evaluated with a number of different analytical tools to identify genes that might serve as markers consistently expressed by endothelial cells in different size vessels in various tissues, as described in Example 4.

Identified Endothelial Cell Specific Genes. The Statistical Analysis of Microarrays (SAM), which rigorously scores differences in gene expression through consideration of the variance exhibited by each gene on the array (Tusher et al. 2001), was employed to refine the sets of genes identified as described in Examples 1 and 2. To provide a rigid criteria for endothelial cell-specific expression, the false detection rate (FDR) was set to 0.0, minimizing the detection of insignificant differences in gene expression (Tusher et al. 2001). The genes that were identified as endothelial cell specific included well characterized endothelial markers such as CD31, VE-cadherin, multimerin, the KDR vascular endothelial cell receptor, and von Willebrand factor (FIG. 1, Table 1). These genes showed a relative expression between endothelial cells and non-endothelial cells that varied between 55-fold and 5-fold. Additional molecules known to be preferentially expressed in endothelial cells were also identified, including endothelial differentiation sphingolipid G-protein coupled receptor (EDG-1), melanoma adhesion receptor (MCAM), endothelin-1, ICAM2, protein C receptor, thymosin beta 4, and plasminogen activator inhibitor type I. The detection of these molecules validated the methodology employed. Interestingly, a number of genes that have been characterized in the context of other tissues and cell types were identified, including netrin 4 precursor, cyclic AMP-dependent protein kinase, bone morphogenetic protein 6, manic fringe, matrix gla protein, and hedgehog interacting protein. While clearly not specific for endothelial cells in vivo, these genes did show higher levels of expression in the endothelial lineage (10-fold to 5-fold), suggesting some differential requirement for their biological function in this cell-type and indicating their utility for identifying and quantifying endothelial cells, imaging endothelial vasculature, etc.

A variety of genes that have been previously characterized by were not known to be differentially expressed in endothelial cells were identified. These genes are listed in Table 2 (Group II genes).

A number of genes that have been previously uncharacterized or minimally characterized were identified as differentially expressed in endothelial cells. These genes are listed in Table 3 (Group III genes). Certain of these genes displayed some sequence similarity with known genes, including ESTs similar to fibulin and ras. Among the most differentially expressed genes were a large group that had been characterized only as ESTs with no specific annotation, and included AA410298, AA256482, and AI422298. A final group of genes were recognized only as hypothetical transcripts through analysis of the human genome, and these included AA004368 and AW772163. Interestingly, a proportionately greater percentage of clones identified through this SAM analysis as showing endothelial cell-preferential expression came from the database mining geneset as opposed to those from subtraction cloning.

FIG. 1A shows a heatmap organized with individual hybridizations arranged along the x-axis, with relative ratios of expression (cellular RNA compared to HUVEC) indicated by color. Color intensity is scaled within each row, so that the highest expression corresponds to bright yellow, and the lowest expression corresponds to bright blue. In some cases, individual genes were represented more than once on the microarray due to redundancy between the genesets, and the corresponding features were handled independently in the data analysis. For this reason, they appear multiple times on the heat map in FIG. 1. Also of note, this statistical analysis did not find genes that were consistently expressed at lower levels by endothelial cells as a group compared to the non-endothelial cells as a group.

These data were also evaluated with a Gaussian error model (GER), which measures the overlap between Gaussian curves as a ranking function, and an analogous gene list was generated through this approach. The separation of signals between the different endothelial and non-endothelial cells is shown for the well known endothelial-specific marker CD31, as well as two novel ESTs identified through this work (FIG. 1B). In each graph the x-axis represents log (red to green ratios). The colored crosses indicate actual data obtained from the respective classes (i.e., endothelial and non-endothelial cell types), and the curves represent best Gaussian fits to the actual data. The threshold indicated represents the intersection of the two curves. The highlighted region is the Bayesean probability of error when using the curves for classification into the two depicted classes.

### Example 4

### Identification of Vascular Bed Specific Genes

Methods

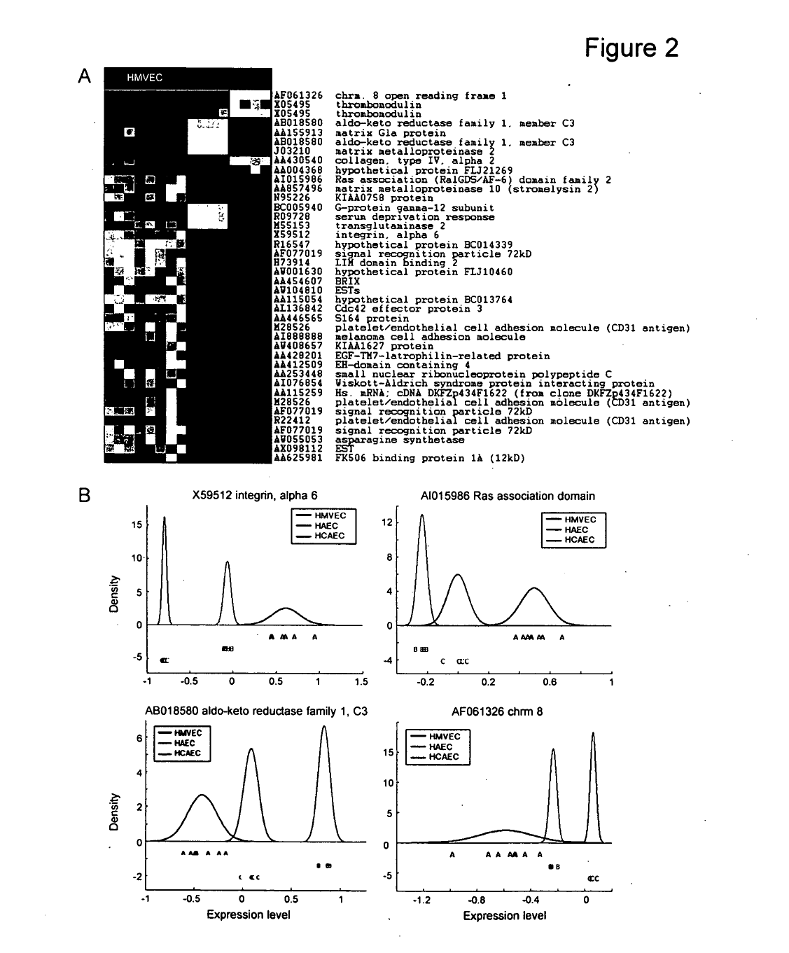

To investigate the issue of differences in gene expression exhibited by endothelial cells depending on their association with different sizes and types of vessels as well as tissues of origin, analyses were conducted to identify differences in gene expression between the HAEC, HCAEC, and the HMVEC. Analysis methods included the Gaussian error model for two-way and multiple comparisons, and SAM for two-way comparisons. In particular, in order to identify genes that are differentially expressed in HAEC, HCAEC, and HMVEC we computed a Gaussian error score applied to three classes corresponding to the three different endothelial cell types for each gene (Ben-Dor et al. 2001). FIG. 2A shows the 40 top-scoring genes grouped by pattern of expression observed in these three classes. Genes in the top group have the highest expression in HCAEC and the lowest expression in HMVEC. In the next group, genes have the highest expression in HAEC, and the lowest in HMVEC. The last group shows genes with the highest expression in HMVEC and the lowest expression in HCAEC. FIG. 2B presents Gaussian distribution plots for six representative genes, showing the separation of signals between the different cell types.

Differentially expressed genes among the different endothelial cell types included thrombomodulin, aldo-keto reductase family member 3, Ras association domain family 2, and matrix metalloproteinase 10, as well as a number of ESTs and hypothetical proteins deduced from the human genome sequence. When the analyses were focused still further, a large number of markers were identified that distinguished between the two types of large vessel endothelial cells, the HAEC and the HCAEC. FIG. 3A shows a portion of the genes found to be preferentially expressed in HAEC (lower panel) and HCAEC (upper panel). FIG. 3B shows genes that were identified as being preferentially expressed at higher levels (lower panel) and lower levels (upper panel) in HMVEC compared to other endothelial cell types.

Representative genes expressed at higher levels in HAEC included those encoding factors involved in coagulation, including multimerin, thrombospondin, tissue factor pathway inhibitor, and von Willebrand factor. Representative genes expressed at higher levels in HCAEC included the chemokine mononuclear cell attractant cytokine (MCP-1) and the endothelial cell-selective protein kinase A scaffold protein gravin (Grove et al. 1994). There were differences in gene expression for extracellular matrix factors, including the basement membrane protein collagen type IV alpha 2, and the matrix gla protein that regulates calcification of matrix.

Since microvascular endothelial cells have markedly different functions from those lining conduit vessels, the specific expression profile of this cell type was investigated. When microvascular cells were compared to all other endothelial cell types, employing the Gaussian error model and SAM, several hundred genes were found to be preferentially expressed in HMVEC. Approximately 30 such genes (represented by 40 clones) are shown in the heatmap in FIG. 3B. Examination of this set of genes provided potential insights into the unique functions of the microvasculature. For example, selective expression of the vascular endothelial cell growth factor receptor KDR was detected by SAM in the HMVEC (data not shown). This result was consistent with known physiology, since the angiogenic program activated by this receptor is mediated at the level of the microcirculation. Differences in expression of factors that mediate cytoplasmic signaling pathways were also evident, and include PAK2 and cdc42 effector protein 3. Many of the known genes identified through this analysis encode proteins that have not been studied in the context of endothelial cell biology, and those genes found to be more highly expressed in HMVEC included LIM binding domain 2, glia maturation factor gamma, thioredoxin reductase, integrin alpha 6 subunit, pre-B cell colony-enhancing factor. A small number of genes were expressed at lower levels in HMVEC compared to all other endothelial cell types, and these included matrix metalloproteinase 2, matrix gla protein, and thrombospondin, as shown in FIG. 3B.

### Example 5

### Validation of Selected Set of Endothelial Cell Specific Genes

Materials and Methods

Northern blot analysis. For each of the nine different cell types, ten micrograms of total RNA were electrophoresed on a 1% formaldehyde agarose gels and transferred onto nylon membranes. The RNA was immobilized on the membranes by baking at 80° C. for 1 hour. Full-length cDNA probes for each corresponding EST were radiolabeled with ³²P-dCTP by random priming. Blots were hybridized at 42° C. for 16 h in the presence of 48% formamide and 10% dextran sulfate. After hybridization, the membranes were washed at high stringency conditions, 65° C. with 0.2×SSC buffer and 0.5% SDS. Visualization was achieved by exposure to Kodak Biomax MS film (Eastman Kodak Co., Rochester, N.Y.).

In situ hybridization. For mouse embryo in situ hybridization, mouse orthologs of the putative endothelial specific ESTs were identified through Unigene, and obtained from Research Genetics. The whole mount in situs of mouse embryo at embryonic day (E) 8.5 and 9.5 were done according to Herique et al. (Henrique et al. 1995). Briefly, digoxigenin-labelled probe were transcribed from linearlized cDNA template and applied to embryos digested with proteinase K. Then embryos were incubated with BM purple substrate and images were taken with a CCD digital camera.

Results

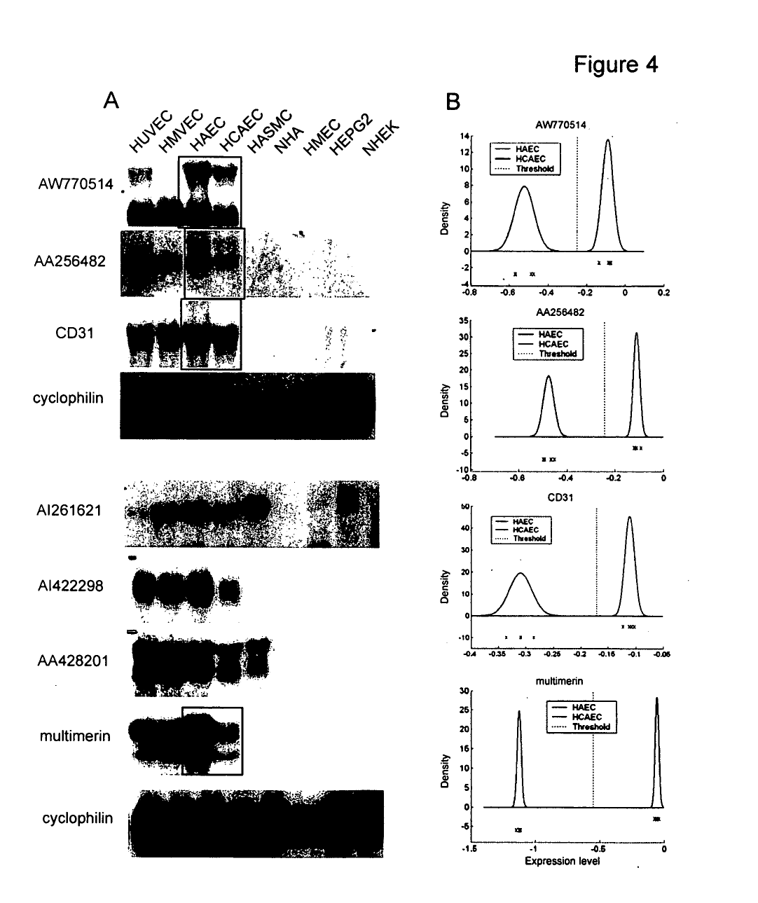

To confirm the microarray methodology and data analysis, a number of RNA blot experiments were conducted. ESTs AW770514, AA256482, AI261621, AI422298, AA428201 were chosen, as well as positive controls CD31 and multimerin (FIG. 4A). ESTs AW770514, AA256482, and AI422298 are highly endothelial restricted, while AI261621 and AA428201 show expression in smooth muscle cells as well. Comparison was made between the Northern blot data and the quantitative information provided by the array experiments as visualized with Gaussian curves (FIG. 4B). Each of the ESTs was found to be primarily endothelial cell-specific, with virtually no detectable expression in the non-endothelial cell lanes. Notable exceptions were AI261621 and AA428201, which showed significant hybridization to the HASMC RNA sample. Even after correcting for differences in RNA loading between the different lanes, differences in hybridization signal were observed between the different endothelial cell types for some genes. Multimerin to a great extent and CD31 to a lesser extent showed more hybridization to the HAEC RNA sample, and the ESTs AI422298 showed a single band with greater hybridization to the HAEC and AW770514 had two specific bands with both showing somewhat greater hybridization with the HAEC RNA. These differences correlate with the data obtained on the microarray (see, for example, FIG. 3A). EST AI261621 showed less hybridization to HUVEC RNA than the other genes. The separation of signals between the HAEC and HCAEC cell types is shown with Gaussian distribution curves for four of the genes evaluated by Northern blot (FIG. 4B).

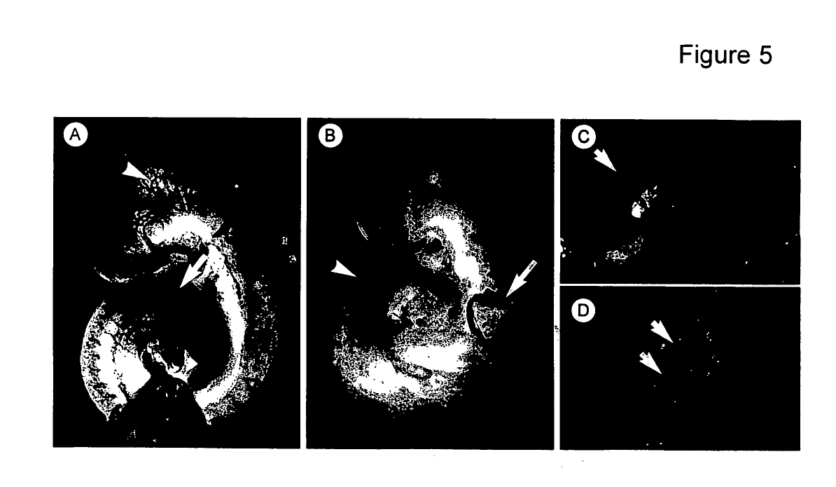

To evaluate in vivo expression of ESTs identified as endothelial cell-specific, in situ hybridization was performed with staged mouse embryos to determine whether these genes might be expressed in early stages of angiogenic blood vessel development (FIG. 5). Of the ten genes studied, five exhibited unique embryonic endothelial cell expression patterns. The patterns varied from expression in all endothelial cells, as observed for one of the ESTs that was determined to represent the AA4 gene (FIG. 5A), to expression in a restricted group of endothelial cells (Jordan et al. 1990). For instance, the mouse ortholog of human EST AW772163 revealed expression restricted to the vitelline veins and aorta, but also showed a temporal expression pattern in the somites (FIG. 5B). The mouse ortholog of human EST W81545 revealed highly restricted expression in the developing blood vessels in the brain and the yolk sac (FIG. 5C).

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

### REFERENCES

Abe, M. and Y. Sato. 2001. cDNA microarray analysis of the gene expression profile of VEGF-activated human umbilical vein endothelial cells. Angiogenesis 4: 289-298.
Auerbach, R., L. Alby, L. W. Morrissey, M. Tu, and J. Joseph. 1985. Expression of organ-specific antigens on capillary endothelial cells. Microvasc Res 29: 401-411.
Bell, S. E., A. Mavila, R. Salazar, K. J. Bayless, S. Kanagala, S. A. Maxwell, and G. E. Davis. 2001. Differential gene expression during capillary morphogenesis in 3D collagen matrices: regulated expression of genes involved in basement membrane matrix assembly, cell cycle progression, cellular differentiation and G-protein signaling. J Cell Sci 114: 2755-2773.
Ben-Dor, A., L. Bruhn, N. Friedman, I. Nachman, M. Schummer, and Z. Yakhini. 2000. Tissue classification with gene expression profiles. J Comput Biol 7: 559-583.
Ben-Dor, A., N. Friedman, and Z. Yakhini. 2001. Class discovery in gene expression data. In Proceedings of the Fifth International Conference on Computational Biology, pp. 31-38.
Bittner, M., P. Meltzer, Y. Chen, Y. Jiang, E. Seftor, M. Hendrix, M. Radmacher, R. Simon, Z. Yakhini, A. Ben-Dor, N. Sampas, E. Dougherty, E. Wang, F. Marincola, C. Gooden, J. Lueders, A. Glatfelter, P. Pollock, J. Carpten, E. Gillanders, D. Leja, K. Dietrich, C. Beaudry, M. Berens, D. Alberts, and V. Sondak. 2000. Molecular classification of cutaneous malignant melanoma by gene expression profiling. Nature 406: 536-540.
Chakravarti, L. and J. Roy. 1967. Handbook of Methods of Applied Statistics, Volume 1. John Wiley & Sons, New York, New York.
Chen, B. P., Y. S. Li, Y. Zhao, K. D. Chen, S. Li, J. Lao, S. Yuan, J. Y. Shyy, and S. Chien. 2001. DNA microarray analysis of gene expression in endothelial cells in response to 24-h shear stress. Physiol Genomics 7: 55-63.
Dekker, R. J., S. van Soest, R. D. Fontijn, S. Salamanca, P. G. de Groot, E. VanBavel, H. Pannekoek, and A. J. Horrevoets. 2002. Prolonged fluid shear stress induces a distinct set of endothelial cell genes, most specifically lung Kruppel-like factor (KLF2). Blood 100: 1689-1698.
Diatchenko, L., Y. F. Lau, A. P. Campbell, A. Chenchik, F. Moqadam, B. Huang, S. Lukyanov, K. Lukyanov, N. Gurskaya, E. D. Sverdlov, and P. D. Siebert. 1996. Suppression subtractive hybridization: a method for generating differentially regulated or tissue-specific cDNA probes and libraries. Proc Natl Acad Sci USA 93: 6025-6030.
Gale, N. W. and G. D. Yancopoulos. 1999. Growth factors acting via endothelial cell-specific receptor tyrosine kinases: VEGFs, angiopoietins, and ephrins in vascular development. Genes Dev 13: 1055-1066.
Garcia-Cardena, G., J. Comander, K. R. Anderson, B. R. Blackman, and M. A. Gimbrone, Jr. 2001. Biomechanical activation of vascular endothelium as a determinant of its functional phenotype. Proc Natl Acad Sci USA 98: 4478-4485.
Gerritsen, M. E., R. Soriano, S. Yang, G. Ingle, C. Zlot, K. Toy, J. Winer, A. Draksharapu, F. Peale, T. D. Wu, and P. M. Williams. 2002. In silico data filtering to identify new angiogenesis targets from a large in vitro gene profiling data set. Physiol Genomics 10:13-20.
Grove, B. D., R. Bowditch, T. Gordon, G. del Zoppo, and M. H. Ginsberg. 1994. Restricted endothelial cell expression of gravin in vivo. Anat Rec 239: 231-242.
Henrique, D., J. Adam, A. Myat, A. Chitnis, J. Lewis, and D. Ish-Horowicz. 1995. Expression of a Delta homologue in prospective neurons in the chick. Nature 375:787-790.
Hirata, K., H. L. Dichek, J. A. Cioffi, S. Y. Choi, N. J. Leeper, L. Quintana, G. S. Kronmal, A. D. Cooper, and T. Quertermous. 1999. Cloning of a unique lipase from endothelial cells extends the lipase gene family. J Biol Chem 274: 14170-14175.
Hishiki, T., S. Kawamoto, S. Morishita, and K. Okubo. 2000. BodyMap: a human and mouse gene expression database. Nucleic Acids Res 28: 136-138.
Hollander, M. and D. A. Wolfe. 1973. Nonparametric Statistical Methods. John Wiley & Sons, New York, New York.
Horrevoets, A. J., R. D. Fontijn, A. J. van Zonneveld, C. J. de Vries, J. W. ten Cate, and H. Pannekoek. 1999. Vascular endothelial genes that are responsive to tumor necrosis factor-alpha in vitro are expressed in atherosclerotic lesions, including inhibitor of apoptosis protein-1, stannin, and two novel genes. Blood 93: 3418-3431.
Huminiecki, L. and R. Bicknell. 2000. In silico cloning of novel endothelial-specific genes. Genome Res 10: 1796-1806.
Jordan, C. T., J. P. McKeam, and I. R. Lemischka. 1990. Cellular and developmental properties of fetal hematopoietic stem cells. Cell 61: 953-963.
Kallmann, B. A., S. Wagner, V. Hummel, M. Buttmann, A. Bayas, J. C. Tonn, and P. Rieckmann. 2002. Characteristic gene expression profile of primary human cerebral endothelial cells. Faseb J 16: 589-591.
Kawamoto, S., J. Yoshii, K. Mizuno, K. Ito, Y. Miyamoto, T. Ohnishi, R. Matoba, N. Hori, Y. Matsumoto, T. Okumura, Y. Nakao, H. Yoshii, J. Arimoto, H. Ohashi, H. Nakanishi, I. Ohno, J. Hashimoto, K. Shimizu, K. Maeda, H. Kuriyama, K. Nishida, A. Shimizu-Matsumoto, W. Adachi, R. Ito, S. Kawasaki, and K. S. Chae. 2000. BodyMap: a collection of 3′ ESTs for analysis of human gene expression information. Genome Res 10:1817-1827.
Loftus, S. K., Y. Chen, G. Gooden, J. F. Ryan, G. Birznieks, M. Hilliard, A. D. Baxevanis, M. Bittner, P. Meltzer, J. Trent, and W. Pavan. 1999. Informatic selection of a neural crest-melanocyte cDNA set for microarray analysis. Proc Natl Acad Sci USA 96:9277-9280.
MacDonald, T. J., K. M. Brown, B. LaFleur, K. Peterson, C. Lawlor, Y. Chen, R. J. Packer, P. Cogen, and D. A. Stephan. 2001. Expression profiling of medulloblastoma: PDGFRA and the RAS/MAPK pathway as therapeutic targets for metastatic disease. Nat Genet 29: 143-152.
Mayanil, C. S., D. George, L. Freilich, E. J. Miljan, B. Mania-Famell, D. G. McLone, and E. G. Bremer. 2001. Microarray analysis detects novel Pax3 downstream target genes. J Biol Chem 276: 49299-49309.
McCormick, S. M., S. G. Eskin, L. V. McIntire, C. L. Teng, C. M. Lu, C. G. Russell, and K. K. Chittur. 2001. DNA microarray reveals changes in gene expression of shear stressed human umbilical vein endothelial cells. Proc Natl Acad Sci USA 98: 8955-8960.
Murakami, T., C. Mataki, C. Nagao, M. Umetani, Y. Wada, M. Ishii, S. Tsutsumi, T. Kohro, A. Saiura, H. Aburatani, T. Hamakubo, and T. Kodama. 2000. The gene expression profile of human umbilical vein endothelial cells stimulated by tumor necrosis factor alpha using DNA microarray analysis. J Atheroscler Thromb 7: 39-44.
Peale, F. V., Jr. and M. E. Gerritsen. 2001. Gene profiling techniques and their application in angiogenesis and vascular development. J Pathol 195: 7-19.
Pepper, M. S. 1997. Transforming growth factor-beta: vasculogenesis, angiogenesis, and vessel wall integrity. Cytokine Growth Factor Rev 8: 21-43.
Perou, C. M., T. Sorlie, M. B. Eisen, M. van de Rijn, S. S. Jeffrey, C. A. Rees, J. R. Pollack, D. T. Ross, H. Johnsen, L. A. Akslen, 0. Fluge, A. Pergamenschikov, C. Williams, S. X. Zhu, P. E. Lonning, A. L. Borresen-Dale, P. O. Brown, and D. Botstein. 2000. Molecular portraits of human breast tumours. Nature 406: 747-752.
Pilpel, Y., P. Sudarsanam, and G. M. Church. 2001. Identifying regulatory networks by combinatorial analysis of promoter elements. Nat Genet 29: 153-159.
Pomeroy, S. L., P. Tamayo, M. Gaasenbeek, L. M. Sturla, M. Angelo, M. E. McLaughlin, J. Y. Kim, L. C. Goumnerova, P. M. Black, C. Lau, J. C. Allen, D. Zagzag, J. M. Olson, T. Curran, C. Wetmore, J. A. Biegel, T. Poggio, S. Mukherjee, R. Rifkin, A. Califano, G. Stolovitzky, D. N. Louis, J. P. Mesirov, E. S. Lander, and T. R. Golub. 2002. Prediction of central nervous system embryonal tumour outcome based on gene expression. Nature 415: 436-442.
Rader, D. J. and M. Jaye. 2000. Endothelial lipase: a new member of the triglyceride lipase gene family. Curr Opin Lipidol 11: 141-147.
Rickman, D. S., M. P. Bobek, D. E. Misek, R. Kuick, M. Blaivas, D. M. Kumit, J. Taylor, and S. M. Hanash. 2001. Distinctive molecular profiles of high-grade and low-grade gliomas based on oligonucleotide microarray analysis. Cancer Res 61: 6885-6891.
Storch, K. F., 0. Lipan, I. Leykin, N. Viswanathan, F. C. Davis, W. H. Wong, and C. J. Weitz. 2002. Extensive and divergent circadian gene expression in liver and heart. Nature 417: 78-83.
Topper, J. N., J. Cai, Y. Qiu, K. R. Anderson, Y. Y. Xu, J. D. Deeds, R. Feeley, C. J. Gimeno, E. A. Woolf, O. Tayber, G. G. Mays, B. A. Sampson, F. J. Schoen, M. A. Gimbrone, Jr., and D. Falb. 1997. Vascular MADs: two novel MAD-related genes selectively inducible by flow in human vascular endothelium. Proc Natl Acad Sci USA 94: 9314-9319.
Tusher, V. G., R. Tibshirani, and G. Chu. 2001. Significance analysis of microarrays applied to the ionizing radiation response. Proc Natl Acad Sci USA 98: 5116-5121.
van't Veer, L. J., H. Dai, M. J. van de Vijver, Y. D. He, A. A. Hart, M. Mao, H. L. Peterse, K. van der Kooy, M. J. Marton, A. T. Witteveen, G. J. Schreiber, R. M. Kerkhoven, C. Roberts, P. S. Linsley, R. Bemards, and S. H. Friend. 2002. Gene expression profiling predicts clinical outcome of breast cancer. Nature 415: 530-536.
Weston, G. C., I. Haviv, and P. A. Rogers. 2002. Microarray analysis of VEGF-responsive genes in myometrial endothelial cells. Mol Hum Reprod 8: 855-863.
Zhang, S., I. N. Day, and S. Ye. 2001. Microarray analysis of nicotine-induced changes in gene expression in endothelial cells. Physiol Genomics 5: 187-192.
Zhao, B., R. A. Bowden, S. A. Stavchansky, and P. D. Bowman. 2001. Human endothelial cell response to gram-negative lipopolysaccharide assessed with cDNA microarrays. Am J Physiol Cell Physiol 281: C1587-1595.
Zhou, J., Y. Jin, Y. Gao, H. Wang, G. Hu, Y. Huang, Q. Chen, M. Feng, and C. Wu. 2002. Genomic-scale analysis of gene expression profiles in TNF-alpha treated human umbilical vein endothelial cells. Inflamm Res 51: 332-341.

1. A composition comprising:
a targeting agent conjugated to a functional moiety, wherein the targeting agent selectively binds to a VECSM Group II or Group III polypeptide.

2. The composition of claim 1, wherein the polypeptide is a VECSM Group III polypeptide.

3. The composition of claim 1, wherein the targeting agent comprises an antibody, an antigen-binding antibody fragment, or a ligand that specifically binds to the polypeptide.

4. The composition of claim 1, wherein the functional moiety comprises a therapeutic or cytotoxic agent.

5. The composition of claim 1, wherein the functional moiety comprises an angiogenesis inhibitor or a stimulator of angiogenesis.

6. The composition of claim 1, wherein the functional moiety comprises a radiosensitizing agent or an imaging agent.

7. The composition of claim 1, wherein the agent is a paramagnetic, radioactive or fluorogenic ion.

8. The composition of claim 7, wherein the agent is selected from the group consisting of: chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), europium, and erbium (III), iodine¹²³, technetium^{99m}, indium¹¹¹, rhenium¹⁸⁸, rhenium¹⁸⁶, copper⁶⁷, iodine¹³¹, yttrium⁹⁰, iodine¹²⁵, astatine²¹¹, and gallium⁶⁷, lanthanum (III), gold (III), lead (II), and bismuth (III).

9. An agent that reduces expression of a VECSM Group II or Group III polynucleotide.

10. The agent of claim 9, wherein the agent comprises an siRNA or shRNA targeted to a VECSM Group II or Group III polynucleotide.

11. The agent of claim 9, wherein the agent comprises an antisense molecule that specifically binds to and inhibits a VECSM Group II or Group III polynucleotide.

12. The agent of claim 9, wherein the agent comprises a ribozyme that cleaves a VECSM Group II or Group III polynucleotide.

13. A method of inhibiting expression of a VECSM Group II or Group III polypeptide in a cell or a subject comprising delivering the agent of claim 9 to the cell or subject.

14. A method of treating a condition comprising steps of:
(i) providing a subject in need of treatment for the condition; and
(ii) administering a pharmaceutical composition comprising the agent of claim 9 to the subject, thereby alleviating the condition.

15. The method of claim 14, wherein the condition is associated with inappropriate or excessive vascular endothelial growth.

16. The method of claim 14, wherein the condition is associated with a reduced or inadequate blood supply.

17. An antibody or antibody fragment that specifically binds to a VECSM Group II or Group III polypeptide, wherein the antibody detects expression of the polypeptide in endothelial cells.

18. A method of treating a condition comprising steps of:
(i) providing a subject in need of treatment for the condition; and
(ii) administering a pharmaceutical composition comprising the antibody of claim 17 to the subject, thereby alleviating the condition.

19. The method of claim 18, wherein the condition is associated with inappropriate or excessive vascular endothelial growth.

20. The method of claim 18, wherein the condition is associated with a reduced or inadequate blood supply.

21. A method for detecting or quantifying vascularization or angiogenic activity in a biological sample or subject comprising:
determining the level of expression of a naturally occurring VECSM Group II or Group III polynucleotide in the biological sample or subject, or determining the level of expression of a naturally occurring polypeptide encoded by such a polynucleotide in the biological sample or subject.

22. The method of claim 21, wherein the biological sample is a tumor sample.

23. A method of imaging angiogenesis, vasculature, or body tissue comprising endothelial cells in a biological sample or subject, comprising steps of:
(i) administering to the biological sample or subject an effective amount of a targeting agent that specifically binds to a VECSM Group II or Group III polypeptide, wherein the targeting agent is linked to a finctional moiety that enhances detectability of endothelial cells by an imaging procedure; and
(ii) subjecting the biological sample or subject to the imaging procedure.

24. The method of claim 23, wherein the subject suffers from a condition associated with excessive or inappropriate vascular tissue or a condition associated with reduced or inadequate blood supply.

25. A method of targeting a molecule selectively to an endothelial cell or to vascular endothelium in a subject comprising steps of:
(i) conjugating the molecule to an antibody or ligand that specifically binds to a VECSM Group II or Group III polypeptide to form a conjugate or associating the molecule with a delivery vehicle, wherein the delivery vehicle comprises a targeting agent that specifically binds to a VECSM Group II or III polypeptide; and
(ii) administering the conjugate or the delivery vehicle with the associated molecule to the cell or subject.

26. A method of inhibiting angiogenesis or of inhibiting tumor growth or tumor survival in a subject comprising steps of:
(a) providing a subject having a condition characterized by excessive or inappropriate angiogenesis or having a tumor; and
(b) administering a composition comprising
(i) a targeting agent that specifically binds to a VECSM Group II or Group III polypeptide; and
(ii) a functional moiety, wherein the functional moiety comprises an angiogenesis inhibitor or a cytotoxic moiety, to the subject, thereby inhibiting angiogenesis, tumor growth, tumor metastasis, or tumor survival in the subject..

27. A method of stimulating angiogenesis in a subject comprising steps of:
(a) providing a subject having a condition characterized by an inadequate blood supply to one or more organs or tissues; and
(b) administering a composition comprising
(i) a targeting agent that specifically binds to a VECSM Group II or Group III polypeptide; and
(ii) a functional moiety, wherein the functional moiety comprises a stimulator of angiogenesis, to the subject, thereby stimulating angiogenesis in the subject.

28. A method for identifying a compound comprising steps of:
(i) providing a biological sample comprising cells that express a VECSM Group II or Group III polypeptide;
(ii) contacting the cells with the compound; and
(iii) determining whether the level of expression or activity of the VECSM polynucleotide or polypeptide in the presence of the compound is increased or decreased relative to the level of expression or activity of the VECSM polynucleotide or polypeptide in the absence of the compound; and
(iv) identifying the compound as a modulator of the VECSM polynucleotide or polypeptide if the level of expression or activity of the VECSM polynucleotide or polypeptide is higher or lower in the presence of the compound relative to its level of expression or activity in the absence of the compound.

29. A compound identified according to the method of claim 28.

30. A method of treating a subject suffering from a disease or clinical condition comprising steps of:
(i) providing a subject suffering from the disease or clinical condition associated with inappropriate or excessive angiogenesis or vascular endothelial growth; and
(ii) administering the compound of claim 29 to the subject.

31. The method of claim 30, wherein the condition is associated with inappropriate or excessive vascular endothelial growth.

32. The method of claim 30, wherein the condition is associated with a reduced or inadequate blood supply.
